SEQUENCE LISTING (175 sequences)

SEQ ID NO: 1 - protein, 162 aa, Homo sapiens
  human IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 267 aa, Homo sapiens
  IL15Ra
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 3 - protein, 551 aa, Homo sapiens
  human IL2Rb
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 4 - protein, 669 aa, Artificial Sequence
  Construct A-M123
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGSISSGLLSGRSDNHGGGSSGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 669 aa, Artificial Sequence
  Construct A-M135
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGSIPVSLRSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 668 aa, Artificial Sequence
  Construct A-M140
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGSSGGSGGSGGSGGGSGGGGSGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 669 aa, Artificial Sequence
  Construct A-M145
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGSVPLSLYSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 669 aa, Artificial Sequence
  Construct A-M175
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSISSGLLSGRSDNHGGGSSGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 669 aa, Artificial Sequence
  Construct A-M176
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 670 aa, Artificial Sequence
  Construct A-M177
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGSVPLSLYSGWRSGGSGGGGSGSGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 671 aa, Artificial Sequence
  Construct A-M178
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGSVPLSLYSGRSASGGSGGGGSGSGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 669 aa, Artificial Sequence
  Construct A-M207
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWISLETLTPDTQYEFQVRVKPLQGEFTTWSPWSQPLAFRTKPAALGKDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 644 aa, Artificial Sequence
  Construct A-M231
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPEENLRLMAPISLQVVHVETHRCNISWEISQASHYFERHLEFEARTLSPGHTWEEAPLLTLKQKQEWICLETLTPDTQYEFQVRVKPLQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 669 aa, Artificial Sequence
  Construct A-M233
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGSIPVSLRSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 437 aa, Artificial Sequence
  Construct A-M234
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGSIPVSLRSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 16 - protein, 674 aa, Artificial Sequence
  Construct A-M238
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWISLETLTPDTQYEFQVRVKPLQGEFTTWSPWSQPLAFRTKPAALGKDGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 679 aa, Artificial Sequence
  Construct A-M239
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWISLETLTPDTQYEFQVRVKPLQGEFTTWSPWSQPLAFRTKPAALGKDGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 649 aa, Artificial Sequence
  Construct A-M240
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPEENLRLMAPISLQVVHVETHRCNISWEISQASHYFERHLEFEARTLSPGHTWEEAPLLTLKQKQEWICLETLTPDTQYEFQVRVKPLQGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 665 aa, Artificial Sequence
  Construct A-M241
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 658 aa, Artificial Sequence
  Construct A-M243
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 669 aa, Artificial Sequence
  Construct A-M244
ITCPPPMSVEHADIWVKSYSLYSRERYICNSGFKRKAGTSSLTECVLNKATNVAHWTTPSLKCIRDPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWITLETLTPDTQYEFQVRVKPLQGEFTTWSPWSQPLAFRTKPAALGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 673 aa, Artificial Sequence
  Construct A-M245
ITCPPPMSVEHADIWVKSYSLYSRERYICNSGFKRKAGTSSLTECVLNKATNVAHWTTPSLKCIRDPALVHQRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWITLETLTPDTQYEFQVRVKPLQGEFTTWSPWSQPLAFRTKPAGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 657 aa, Artificial Sequence
  Construct A-M246
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWITLETLTPDTQYEFQVRVKPLQGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 677 aa, Artificial Sequence
  Construct A-M247
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWITLETLTPDTQYEFQVRVKPLQGEFTTWSPWSQPLAFRTKPAGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 677 aa, Artificial Sequence
  Construct A-M248
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWITLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 682 aa, Artificial Sequence
  Construct A-M249
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWITLETLTPDTQYEFQVRVKPLQGEFTTWSPWSQPLAFRTKPAGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 687 aa, Artificial Sequence
  Construct A-M327
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 688 aa, Artificial Sequence
  Construct A-M328
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTAPAPAPEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 688 aa, Artificial Sequence
  Construct A-M329
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWITLETLTPDTQYEFQVRVKPLQGEFTTWSPWSQPLAFRTKPAALGKDTAPAPAPEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 689 aa, Artificial Sequence
  Construct A-M330
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSVPLSLYSGRSASGGSGGGGSGSGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 689 aa, Artificial Sequence
  Construct A-M331
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSVPLSLYSGRSASGGSGGGGSGSGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTEAAAKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 689 aa, Artificial Sequence
  Construct A-M332
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSVPLSLYSGRSASGGSGGGGSGSGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWITLETLTPDTQYEFQVRVKPLQGEFTTWSPWSQPLAFRTKPAALGKDTEAAAKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 358 aa, Artificial Sequence
  Construct B-M43, pairs with M24 (SEQ ID NO34)
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDGGGSSGGSGGSGGGSGGGSLSGRSDNHGGSGNWVNVISDLKKIEDLIQSMHIDATLYSESDVHPSCKVTAMKCFLLEFQVISCESGDASIHDTVENLIILANDSLSSNGNVTESGCKECEELEEKNIKEFLQSFVHIVQMFINTS

SEQ ID NO: 34 - protein, 305 aa, Artificial Sequence
  Construct B-M24
ITCPPPMSVEHADIWVKSYSLYSRERYICNSGFKRKAGTCSLTECVLNKATNVAHWTTPSLKCIRDPALVHQREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 349 aa, Artificial Sequence
  Construct B-M61, pairs with M60 (SEQ ID NO36)
GTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWISLETLTPDTQYEFQVRVKPLQGEFTTWSPWSQPLAFRTKPAALGKDGGGSIPVSLRSGGGGSSGGSGGSGGNWVNVISDLKKIEDLIQSMHIDATLYSESDVHPSCKVTAMKCFLLEFQVISCESGDASIHDTVENLIILANDSLSSNGNVTESGCKECEELEEKNIKEFLQSFVHIVQMFINTS

SEQ ID NO: 36 - protein, 305 aa, Artificial Sequence
  Construct B-M60
ITCPPPMSVEHADIWVKSYSLYSRERYICNSGFKRKAGTCSLTECVLNKATNVAHWTTPSLKCIRDPALVHQREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 352 aa, Artificial Sequence
  Construct B-M62, pairs with M60 (SEQ ID NO38)
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWISLETLTPDTQYEFQVRVKPLQGEFTTWSPWSQPLAFRTKPAALGKDGGGSISSGLLSGRSDNHGGGSSGGSNWVNVISDLKKIEDLIQSMHIDATLYTESDVHPSCKVTAMKCFLLELQVISCESGDASIHDTVENLIILANDSLSSNGNVTESGCKECEELEEKNIKEFLQSFVHIVQMFINTS

SEQ ID NO: 38 - protein, 305 aa, Artificial Sequence; identical to SEQ ID NO: 36
  Construct B-M60

SEQ ID NO: 39 - protein, 678 aa, Artificial Sequence
  Construct C-M101
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDGGGSSGGSGGSGGSGGGSGGGSLSGRSDNHGGSGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 674 aa, Artificial Sequence
  Construct C-M148
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDGGGSSGGSGGSGGIPVSLRSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSGGGGSGGGGSGGGGSGGGSLQITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 687 aa, Artificial Sequence
  Construct C-M174
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDGGGGSSGRIGFLRTAGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 669 aa, Artificial Sequence
  Construct D-M232
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDGGGSIPVSLRSGGGGSSGGSGGSGGITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 43 - protein, 680 aa, Artificial Sequence
  Construct D-M1001
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDGGGSSGGSGGSGGIPVSLRSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 44 - protein, 685 aa, Artificial Sequence
  Construct D-M1002
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDGGGSSGGSGGSGGIPVSLRSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 45 - protein, 690 aa, Artificial Sequence
  Construct D-M1003
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDGGGSSGGSGGSGGIPVSLRSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 46 - protein, 681 aa, Artificial Sequence
  Construct D-M1004
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGSSGGSGGSGGIPVSLRSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 47 - protein, 681 aa, Artificial Sequence
  Construct D-M1005
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWITLETLTPDTQYEFQVRVKPLQGEFTTWSPWSQPLAFRTKPAALGKDTGGGSSGGSGGSGGIPVSLRSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 48 - protein, 681 aa, Artificial Sequence
  Construct D-M1006
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAVQGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWITLETLTPDTQYEFQVRVKPLQGEFTTWSPWSQPLAFRTKPAALGKDTGGGSSGGSGGSGGIPVSLRSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFIQTS

SEQ ID NO: 49 - protein, 669 aa, Artificial Sequence
  Construct E1-MK107. pairs with MH2 (SEQ ID NO 50)
ITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 232 aa, Artificial Sequence
  MH2
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 51 - protein, 678 aa, Artificial Sequence
  Construct E3-MK114, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 227 aa, Artificial Sequence
  Construct E3-MH7
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 53 - protein, 678 aa, Artificial Sequence
  Construct E3-MK115, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFIQTSGGGSIPVSLRSGGGGSSGGSGGSGGGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 687 aa, Artificial Sequence
  Construct E3-MK117, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWITLETLTPDTQYEFQVRVKPLQGEFTTWSPWSQPLAFRTKPAALGKDTGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 687 aa, Artificial Sequence
  Construct E3-MK118, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGAVQGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWITLETLTPDTQYEFQVRVKPLQGEFTTWSPWSQPLAFRTKPAALGKDTGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 687 aa, Artificial Sequence
  Construct E3-MK119, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFIQTSGGGSIPVSLRSGGGGSSGGSGGSGGAVQGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWITLETLTPDTQYEFQVRVKPLQGEFTTWSPWSQPLAFRTKPAALGKDTGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 687 aa, Artificial Sequence
  Construct E3-MK120, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFIQTSGGGSIPVSLRSGGGGSSGGSGGSGGAVQGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWITLETLTPDTQYEFQVRVKPLQGEFTTWSPWSQPLAFRTKPAALGKDTGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 689 aa, Artificial Sequence
  Construct E3-MK121, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSVPLSLYSGRSASGGSGGGGSGSGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWITLETLTPDTQYEFQVRVKPLQGEFTTWSPWSQPLAFRTKPAALGKDTGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 687 aa, Artificial Sequence
  Construct E3-MK123, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 689 aa, Artificial Sequence
  Construct E3-MK124, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSVPLSLYSGRSASGGSGGGGSGSGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 687 aa, Artificial Sequence
  Construct E3-MK125, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWITLETLTPDTQYEFQVRVKPLQGEFTTWSPWSQPLAFRTKPAALGKDTEAAAKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 690 aa, Artificial Sequence
  Construct E3-MK126, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSVPLSLYSGRSASGGSGGGGSGSGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWITLETLTPDTQYEFQVRVKPLQGEFTTWSPWSQPLAFRTKPAALGKDTAPAPAPEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 669 aa, Artificial Sequence
  Construct E2-M111, pairs with MH2 (SEQ ID NO 64)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDGGGSIPVSLRSGGGGSSGGSGGSGGITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 64 - protein, 232 aa, Artificial Sequence; identical to SEQ ID NO: 50
  Construct E2-MH2

SEQ ID NO: 65 - protein, 681 aa, Artificial Sequence
  Construct E2-M2001, pairs with MH7 (SEQ ID NO 52)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWITLETLTPDTQYEFQVRVKPLQGEFTTWSPWSQPLAFRTKPAALGKDTGGGSSGGSGGSGGIPVSLRSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 66 - protein, 681 aa, Artificial Sequence
  Construct E2-M2002, pairs with MH7 (SEQ ID NO 52)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSAVQGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWITLETLTPDTQYEFQVRVKPLQGEFTTWSPWSQPLAFRTKPAALGKDTGGGSSGGSGGSGGIPVSLRSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFIQTS

SEQ ID NO: 67 - protein, 680 aa, Artificial Sequence
  Construct E3-MK136, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSVPLSLYSGRSASGGSGGGGSGSGGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 680 aa, Artificial Sequence
  Construct E3-MK137, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFIQTSGGGSVPLSLYSGRSASGGSGGGGSGSGGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 680 aa, Artificial Sequence; identical to SEQ ID NO: 68
  Construct E3-MK138, pairs with MH7 (SEQ ID NO 52)

SEQ ID NO: 70 - protein, 677 aa, Artificial Sequence
  Construct E3-MK139, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFIQTSGGGSVPLSLYSGRSASGGSGGGGSGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 674 aa, Artificial Sequence
  Construct E3-MK140, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFIQTSGGGSVPLSLYSGRSASGGSGGGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 680 aa, Artificial Sequence
  Construct E3-MK141, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFIQTSGGGSVPLSLYSGRSASGGSGGGGSGSGGTSQFTCFYNSRAQISCVWSQDGALQDTSCQVHAWPDRRRWNQTCELLPVSQASWACNLILGAPDSQKLTTQDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 686 aa, Artificial Sequence
  Construct E3-MK146, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGGSGGGGSGGGGSGGGGSGGGSLQNWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFIQTSGGGSVPLSLYSGRSASGGSGGGGSGSGGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 680 aa, Artificial Sequence
  Construct E3-MK145, pairs with MH8 (SEQ ID NO 75)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFIQTSGGGSVPLSLYSGRSASGGSGGGGSGSGGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 227 aa, Artificial Sequence
  Construct E3-MH8
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 76 - protein, 677 aa, Artificial Sequence
  Construct E3-MK149, pairs with MH8 (SEQ ID NO 75)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFIQTSGGGSVPLSLYSGRSASGGSGGGGSGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 677 aa, Artificial Sequence
  Construct E3-MK150,pairs with MH8 (SEQ ID NO 75)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGQVTESGCKECEELEEKNIKEFLQSFVHIVQMFIQTSGGGSVPLSLYSGRSASGGSGGGGSGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 677 aa, Artificial Sequence
  Construct E3-MK151, pairs with MH8 (SEQ ID NO 75)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGQVTESGCKECEELEEKNIKEFLQSFVHIVQMFIQTSGGGSVPLSLYSGRSASGGSGGGGSGTSQFTCFYNSRAQISCVWSQDGALQDTSCQVHAWPDRRRWKQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 677 aa, Artificial Sequence
  Construct E3-MK152, pairs with MH8 (SEQ ID NO 75)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGQVTESGCKECEELEEKNIKEFLQSFVHIVQMFIQTSGGGSVPLSLYSGRSASGGSGGGGSGTSQFTCFYNSRAQISCVWSQDGALQDTSCQVHAWPDRRRWRQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 677 aa, Artificial Sequence
  Construct E3-MK153, pairs with MH8 (SEQ ID NO 75)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGQVTESGCKECEELEEKNIKEFLQSFVHIVQMFIQTSGGGSVPLSLYSGRSASGGSGGGGSGTSQFTCFYNSRAQISCVWSQDGALQDTSCQVHAWPDRRRWQQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 680 aa, Artificial Sequence
  Construct E3-MK154, pairs with MH8 (SEQ ID NO 75)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGQVTESGCKECEELEEKNIKEFLQSFVHIVQMFIQTSGGGSVPLSLYSGRSASGGSGGGGSGSGGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 677 aa, Artificial Sequence
  Construct E3-MK155, pairs with MH7 (SEQ ID NO 52)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGQVTESGCKECEELEEKNIKEFLQSFVHIVQMFIQTSGGGSVPLSLYSGRSASGGSGGGGSGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 451 aa, Artificial Sequence
  Construct F1-M109, pairs with MH110 (SEQ ID NO 84
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGSIPVSLRSGGGGSSGGSGGSGGITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 84 - protein, 445 aa, Artificial Sequence
  Construct F1-MH110
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 85 - protein, 453 aa, Artificial Sequence
  Construct F1-M2003, pairs with MH2004 (SEQ ID NO86)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSIPVSLRSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 86 - protein, 445 aa, Artificial Sequence
  Construct F1-MH2004
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWITLETLTPDTQYEFQVRVKPLQGEFTTWSPWSQPLAFRTKPAALGKDT

SEQ ID NO: 87 - protein, 453 aa, Artificial Sequence; identical to SEQ ID NO: 85
  Construct F1-M2003, pairs with M2005 (SEQ ID NO88)

SEQ ID NO: 88 - protein, 445 aa, Artificial Sequence
  Construct F1-M2005
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSAVQGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWITLETLTPDTQYEFQVRVKPLQGEFTTWSPWSQPLAFRTKPAALGKDT

SEQ ID NO: 89 - protein, 419 aa, Artificial Sequence
  Construct F2-M005, pairs with MK5 (SEQ ID NO 90)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPEENLRLMAPISLQVVHVETHRCNISWEISQASHYFERHLEFEARTLSPGHTWEEAPLLTLKQKQEWICLETLTPDTQYEFQVRVKPLQ

SEQ ID NO: 90 - protein, 462 aa, Artificial Sequence
  Construct F2-MK5
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSSGGSGGSGGIPVSLRSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPP

SEQ ID NO: 91 - protein, 444 aa, Artificial Sequence
  Construct F2-M2006, pairs with MK5 (SEQ ID NO 90
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 92 - protein, 419 aa, Artificial Sequence
  Construct F3-M006, pairs with MK6 (SEQ ID NO 93)
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPEENLRLMAPISLQVVHVETHRCNISWEISQASHYFERHLEFEARTLSPGHTWEEAPLLTLKQKQEWICLETLTPDTQYEFQVRVKPLQGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 93 - protein, 462 aa, Artificial Sequence
  Construct F3-MK6
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 444 aa, Artificial Sequence
  Construct F3-M2007, pairs with MK6 (SEQ ID NO 93)
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 95 - protein, 584 aa, Artificial Sequence
  Construct G1-M108, pairs with MH4 (SEQ ID NO 96)
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDGGGSIPVSLRSGGGGSSGGSGGSGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 297 aa, Artificial Sequence
  Construct G1-MH4
ITCPPPMSVEHADIWVKSYSLYSRERYICNSGFKRKAGTSSLTECVLNKATNVAHWTTPSLKCIREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 97 - protein, 583 aa, Artificial Sequence
  Construct G2-M112, pairs with MK113 (SEQ ID NO 98)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSIPVSLRSGGGGSSGGSGGSGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 98 - protein, 296 aa, Artificial Sequence
  Construct G2-MK113
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSITCPPPMSVEHADIWVKSYSLYSRERYICNSGFKRKAGTSSLTECVLNKATNVAHWTTPSLKCIR

SEQ ID NO: 99 - protein, 900 aa, Artificial Sequence
  Construct H1-M001, pairs with MH333LC (SEQ ID NO 100)
EVQLVESGGGLVQPGGSLRLSCAVSGFSLTSYGVHWVRQAPGKGLEWVAVIWAGGSTNYADSVKGRFTISKDTSKNTVYLQMNSLRAEDTAVYYCAKPYGTSAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDGGGSSGGSGGSGGIPVSLRSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 100 - protein, 215 aa, Artificial Sequence
  Construct H1-MH333 LC
DIQMTQSPSSLSASVGDRVTITCKASQDVGIVVAWYQQKPGKAPKLLIYWASIRHTGVPSRFSGSGSGTEFTLTISSLQPDDFATYYCQQYSNYPLYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 900 aa, Artificial Sequence
  Construct H2-M002, pairs with MH2 SEQ ID NO 102 and MH333 LC SEQ ID NO100
EVQLVESGGGLVQPGGSLRLSCAVSGFSLTSYGVHWVRQAPGKGLEWVAVIWAGGSTNYADSVKGRFTISKDTSKNTVYLQMNSLRAEDTAVYYCAKPYGTSAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDGGGSSGGSGGSGGIPVSLRSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 102 - protein, 446 aa, Artificial Sequence
  Construct H2-MH2
EVQLVESGGGLVQPGGSLRLSCAVSGFSLTSYGVHWVRQAPGKGLEWVAVIWAGGSTNYADSVKGRFTISKDTSKNTVYLQMNSLRAEDTAVYYCAKPYGTSAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 103 - protein, 673 aa, Artificial Sequence
  Construct K1-MK3, pairs with MH3 (SEQ ID NO 104 and MH333 LC (SEQ ID NO100)
EVQLVESGGGLVQPGGSLRLSCAVSGFSLTSYGVHWVRQAPGKGLEWVAVIWAGGSTNYADSVKGRFTISKDTSKNTVYLQMNSLRAEDTAVYYCAKPYGTSAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGSIPVSLRSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 104 - protein, 665 aa, Artificial Sequence
  Construct K1-MH3
EVQLVESGGGLVQPGGSLRLSCAVSGFSLTSYGVHWVRQAPGKGLEWVAVIWAGGSTNYADSVKGRFTISKDTSKNTVYLQMNSLRAEDTAVYYCAKPYGTSAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWITLETLTPDTQYEFQVRVKPLQGEFTTWSPWSQPLAFRTKPAALGKDT

SEQ ID NO: 105 - protein, 682 aa, Artificial Sequence
  Construct K2-MK4, pairs with MH3 (SEQ ID NO 106) and MH333 LC (SEQ ID NO100)
EVQLVESGGGLVQPGGSLRLSCAVSGFSLTSYGVHWVRQAPGKGLEWVAVIWAGGSTNYADSVKGRFTISKDTSKNTVYLQMNSLRAEDTAVYYCAKPYGTSAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGSSGGSGGSGGIPVSLRSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPP

SEQ ID NO: 106 - protein, 665 aa, Artificial Sequence; identical to SEQ ID NO: 104
  Construct K2-MH3

SEQ ID NO: 107 - protein, 346 aa, Artificial Sequence
  Construct M-MK143, Pair with MK144 (SEQ ID NO 108) and MH7 (SEQ ID NO52)
GTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGSVPLSLYSGRSASGGSGGGGSGSGNWVNVISDLKKIEDLIQSMHIDATLYTESDVHPSCKVTAMKCFLLELQVISCESGDASIHDTVENLIILAQDSLSSNGNVTESGCKECEELEEKNIKEFLQSFVHIVQMFIQTS

SEQ ID NO: 108 - protein, 305 aa, Artificial Sequence
  Construct M-MK144
ITCPPPMSVEHADIWVKSYSLYSRERYICNSGFKRKAGTCSLTECVLNKATNVAHWTTPSLKCIRDPALVHQREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 680 aa, Artificial Sequence
  Construct N-MK142, Pair with MH7 (SEQ ID NO52)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFIQTSGGGSVPLSLYSGRSASGGSGGGGSGSGGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPA

SEQ ID NO: 110 - protein, 73 aa, Artificial Sequence
  Construct P-MK147, Pair with MK148 (SEQ ID NO 111) and MH7 (SEQ ID NO52)
ITCPPPMSVEHADIWVKSYSLYSRERYICNSGFKRKAGTCSLTECVLNKATNVAHWTTPSLKCIRDPALVHQR

SEQ ID NO: 111 - protein, 583 aa, Artificial Sequence
  Construct P-MK148
NWVNVISDLKKIEDLIQSMHIDATLYTESDVHPSCKVTAMKCFLLELQVISCESGDASIHDTVENLIILAQDSLSSNGNVTESGCKECEELEEKNIKEFLQSFVHIVQMFIQTSGGGSVPLSLYSGRSASGGSGGGGSGSGGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 232 aa, Artificial Sequence
  IgGGHG1-Fc, IgHG1 (99-330)
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 232 aa, Artificial Sequence
  IgGGHG1-Fc, IgHG1 (99-330, C103S)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 227 aa, Artificial Sequence
  IgGGHG1-Fc, IgHG1 (104-330)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 231 aa, Artificial Sequence
  IgGGHG1-Fc, IgHG1 (99-329, C103S)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 226 aa, Artificial Sequence
  IgGGHG1-Fc, IgHG1 (104-329)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 232 aa, Artificial Sequence
  IgGGHG1-Fc, IgHG1 (99-330, C103S, S354C, T366W)knob
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 232 aa, Artificial Sequence; identical to SEQ ID NO: 50
  IgGGHG1-Fc, IgHG1 (99-330, C103S, Y349C, T366S, L368A, Y407V) hole

SEQ ID NO: 119 - protein, 231 aa, Artificial Sequence
  IgGGHG1-Fc, IgHG1 (99-329, C103S, S354C, T366W) knob
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - protein, 231 aa, Artificial Sequence
  IgGGHG1-Fc, IgHG1 (99-329, C103S, Y349C, T366S, L368A, Y407V) hole
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 121 - protein, 227 aa, Artificial Sequence
  IgGGHG1-Fc, IgHG1 (104-330, S354C, T366W)knob
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 227 aa, Artificial Sequence; identical to SEQ ID NO: 121
  IgGGHG1-Fc, IgHG1 (104-330, S354C, T366W)knob

SEQ ID NO: 123 - protein, 226 aa, Artificial Sequence
  IgGGHG1-Fc, IgHG1 (104-329, S354C, T366W)knob
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - protein, 226 aa, Artificial Sequence
  IgGGHG1-Fc, IgHG1 (104-329, Y349C, T366S, L368A, Y407V) hole
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 125 - protein, 227 aa, Artificial Sequence [sequence omitted]

SEQ ID NO: 126 - protein, 227 aa, Artificial Sequence; identical to SEQ ID NO: 75
  IgGGHG1-Fc, IGHG1 (104-330, T366S, L368A, Y407V) hole

SEQ ID NO: 127 - protein, 20 aa, Artificial Sequence
  Linker
SGGSGGGGSGGGSGGGGSLQ

SEQ ID NO: 128 - protein, 3 aa, Artificial Sequence
  Linker
GGS

SEQ ID NO: 129 - protein, 4 aa, Artificial Sequence
  Linker
GGGS

SEQ ID NO: 130 - protein, 5 aa, Artificial Sequence
  Linker
GGGGS

SEQ ID NO: 131 - protein, 8 aa, Artificial Sequence
  Linker
GGGSSGGS

SEQ ID NO: 132 - protein, 10 aa, Artificial Sequence
  Linker
GGGGSGGGGS

SEQ ID NO: 133 - protein, 12 aa, Artificial Sequence
  Linker
SGGSGGGGSGSG

SEQ ID NO: 134 - protein, 13 aa, Artificial Sequence
  Linker
GGGSSGGSGGSGG

SEQ ID NO: 135 - protein, 14 aa, Artificial Sequence
  Linker
GGGSSGGSGGSGGS

SEQ ID NO: 136 - protein, 15 aa, Artificial Sequence
  Linker
GGGSSGGSGGSGGSG

SEQ ID NO: 137 - protein, 24 aa, Artificial Sequence
  Linker
GGGSSGGSGGSGGSGGGSGGGGSG

SEQ ID NO: 138 - protein, 25 aa, Artificial Sequence
  Linker
GGGSSGGSGGSGGGSSGGSGGSGGS

SEQ ID NO: 139 - protein, 25 aa, Artificial Sequence
  Linker
GGGSGGGSSGGSGGSGGGGGSSGGS

SEQ ID NO: 140 - protein, 11 aa, Artificial Sequence
  Linker
GGGSSGGSGGG

SEQ ID NO: 141 - protein, 19 aa, Artificial Sequence
  Linker
GGGSSGGSGGSGGGSGGGS

SEQ ID NO: 142 - protein, 4 aa, Artificial Sequence
  Linker
GGSG

SEQ ID NO: 143 - protein, 22 aa, Artificial Sequence
  GGGSSGGSGGSGGSGGGSGGGS
GGGSSGGSGGSGGSGGGSGGGS

SEQ ID NO: 144 - protein, 26 aa, Artificial Sequence
  Linker
SGGGSGGGGSGGGGSGGGGSGGGSLQ

SEQ ID NO: 145 - protein, 15 aa, Artificial Sequence
  Linker
GGGGSGGGGSGGGGS

SEQ ID NO: 146 - protein, 20 aa, Artificial Sequence
  Linker
GGGGSGGGGSGGGGSGGGGS

SEQ ID NO: 147 - protein, 7 aa, Artificial Sequence
  Linker
GGGGSSG

SEQ ID NO: 148 - protein, 24 aa, Artificial Sequence
  Linker
GGGGSGGGGSGGGGSSGGSGGSGG

SEQ ID NO: 149 - protein, 9 aa, Artificial Sequence
  Linker
SGGSGGGGS

SEQ ID NO: 150 - protein, 6 aa, Artificial Sequence
  Linker
SGGSGG

SEQ ID NO: 151 - protein, 13 aa, Artificial Sequence
  Protease activatable moiety
ISSGLLSGRSDNH

SEQ ID NO: 152 - protein, 13 aa, Artificial Sequence
  Protease activatable moiety
ISSGLLSGRSANP

SEQ ID NO: 153 - protein, 8 aa, Artificial Sequence
  Protease activatable moiety
LSGRSDNH

SEQ ID NO: 154 - protein, 8 aa, Artificial Sequence
  Protease activatable moiety
LSGRSANP

SEQ ID NO: 155 - protein, 6 aa, Artificial Sequence
  Protease activatable moiety
PLGLAG

SEQ ID NO: 156 - protein, 8 aa, Artificial Sequence
  Protease activatable moiety
IPVSLRSG

SEQ ID NO: 157 - protein, 8 aa, Artificial Sequence
  Protease activatable moiety
GPQGIAGQ

SEQ ID NO: 158 - protein, 8 aa, Artificial Sequence
  Protease activatable moiety
VPMSMRGG

SEQ ID NO: 159 - protein, 8 aa, Artificial Sequence
  Protease activatable moiety
RPMSMIMG

SEQ ID NO: 160 - protein, 8 aa, Artificial Sequence
  Protease activatable moiety
VPLSLTMG

SEQ ID NO: 161 - protein, 8 aa, Artificial Sequence
  Protease activatable moiety
VPLSLYSG

SEQ ID NO: 162 - protein, 8 aa, Artificial Sequence
  Protease activatable moiety
IPESLRAG

SEQ ID NO: 163 - protein, 10 aa, Artificial Sequence
  Protease activatable moiety
IPVSLRSGWR

SEQ ID NO: 164 - protein, 11 aa, Artificial Sequence
  Protease activatable moiety
IPVSLRSGRSA

SEQ ID NO: 165 - protein, 10 aa, Artificial Sequence
  Protease activatable moiety
VPLSLYSGWR

SEQ ID NO: 166 - protein, 11 aa, Artificial Sequence
  Protease activatable moiety
VPLSLYSGRSA

SEQ ID NO: 167 - protein, 8 aa, Artificial Sequence
  Protease activatable moiety
GAANLVRG

SEQ ID NO: 168 - protein, 8 aa, Artificial Sequence
  Protease activatable moiety
GYAELRMG

SEQ ID NO: 169 - protein, 8 aa, Artificial Sequence
  Protease activatable moiety
MPYDLYHP

SEQ ID NO: 170 - protein, 8 aa, Artificial Sequence
  Protease activatable moiety
RIGFLRTA

SEQ ID NO: 171 - protein, 8 aa, Artificial Sequence
  Protease activatable moiety
ARYRWLTA

SEQ ID NO: 172 - protein, 677 aa, Artificial Sequence
  Construct E3-MK157, pairs with MH8 (SEQ ID NO 75)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFIQTSGGGSVPLSLYSGRSASGGSGGGGSGTSQFTCFYNSRAQISCVWSQDGALQDTSCQVHAWPDRRRWKQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPAGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 677 aa, Artificial Sequence
  Construct N-MK156, pairs with MH8 (SEQ ID NO 75)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGQVTESGCKECEELEEKNIKEFLQSFVHIVQMFIQTSGGGSVPLSLYSGRSASGGSGGGGSGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPA

SEQ ID NO: 174 - protein, 672 aa, Artificial Sequence
  Construct N-MK165, pairs with MH8 (SEQ ID NO 75)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGQVTESGCKECEELEEKNIKEFLQSFVHIVQMFIQTSGGGSVPLSLYSGRSASGGSGGGGSGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPA

SEQ ID NO: 175 - protein, 891 aa, Artificial Sequence
  Construct Q-MK14, pairs with MH2 (SEQ ID NO 102) and MH333 LC (SEQ ID NO 100)
EVQLVESGGGLVQPGGSLRLSCAVSGFSLTSYGVHWVRQAPGKGLEWVAVIWAGGSTNYADSVKGRFTISKDTSKNTVYLQMNSLRAEDTAVYYCAKPYGTSAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILAQNSLSSNGQVTESGCKECEELEEKNIKEFLQSFVHIVQMFIQTSGGGSVPLSLYSGRSASGGSGGGGSGTSQFTCFYNSRAQISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRAQISWEISQASHYFERHLEFEARTLSPGHTWEEAPLLTLKQKQEWISLETLTPDTQYEFQVRVKPLQGEFTTWSPWSQPLAFRTKPA

(19) United States
(12) Patent Application Publication
(10) Pub. No.: US 2023/0357344 A1
(43) Pub. Date: Nov. 9, 2023

(54) Interleukin 15 Constructs and Methods of Use

(71) Applicant: BeiGene, Ltd., Camana Bay, Grand Cayman (KY)

(72) Inventors: Xudong Luan, Beijing (CN); Xuesong Liu, Beijing (CN); Ming Lei, Beijing (CN)

(21) Appl. No.: 18/245,654

(22) PCT Filed: Sep. 16, 2021
(86) PCT No.: PCT/CN2021/118679

§ 371 Date: Mar. 16, 2023

(30) Foreign Application Priority Data
Sep. 16, 2020 (WO) PCT/CN2020/115594
Jul. 15, 2021 (WO) PCT/CN2021/106481
Sep. 1, 2021 (WO) PCT/CN2021/116077

(51) Int. Cl. C07K 14/54; C07K 14/715; A61P 35/00; C07K 16/30

(52) U.S. Cl. CPC C07K 14/5443; C07K 14/7155; A61P 35/00; C07K 16/3076; C07K 2319/30

(57) ABSTRACT

Provided arm IL15 constructs, a pharmaceutical composition comprising said IL15 constructs, and use of the IL15 constructs or the composition for treating a disease, such as cancer, infectious disease or an immune disorder.

### FIELD OF THE DISCLOSURE

Disclosed herein are interleukin 15 (IL15) constructs, as well as methods of use for the treatment of cancer.

### BACKGROUND

IL15 is a cytokine originally described as a T cell growth factor. The cytokine belongs to the four α-helix bundle family, and its receptor consists of two subunits (the IL-2R/IL-15R p and γ chains) responsible for signal transduction. These receptors are expressed for example on activated T cells, and which can be activated with picomolar concentrations of IL15.

As a therapeutic, IL15 shows promise in the activation of T cells, especially CD8+ T cells, however, there are issues with dosing a patent due to the short half-life and rapid clearance of the molecule. Currently, there are no approved uses of recombinant IL-15, although several clinical trials are ongoing.

Thus, there is an unmet need in the art to provide for IL15 constructs that are able to deliver IL15 directly to the tumor microenvironment in a manner that provides for superior delivery of the molecule.

### SUMMARY OF THE DISCLOSURE

The present disclosure is directed to IL15 constructs. In one embodiment the IL15 construct is a bivalent, homodimeric interleukin 15 (IL15) construct comprising from N-terminus to C-terminus:
a) an IL15 receptor alpha (IL15Ra) domain linked to;
b) a first linker (L1) linked to;
c) an IL15 domain, linked to;
d) a second linker (L2) containing a protease activatable moiety linked to;
e) an Interleukin 2 receptor beta (IL2Rb) domain; and
f) an IgG1 Fc region, wherein the bivalent IL15 construct comprises:
(i) a homodimer set forth in SEQ ID NO:4 (M123);
(ii) a homodimer set forth in SEQ ID NO:5 (M135);
(iii) a homodimer set forth in SEQ ID NO:6 (M140);
(iv) a homodimer set forth in SEQ ID NO:7 (M145);
(v) a homodimer set forth in SEQ ID NO:8 (M175);
(vi) a homodimer set forth in SEQ ID NO:9 (M176);
(vii) a homodimer set forth in SEQ ID NO:10 (M177);
(viii) a homodimer set forth in SEQ ID NO:11 (M178);
(ix) a homodimer set forth in SEQ ID NO:12 (M207);
(x) a homodimer set forth in SEQ ID NO:13 (M231);
(xi) a homodimer set forth in SEQ ID NO:14 (M233);
(xii) a homodimer set forth in SEQ ID NO:15 (M234);
(xiii) a homodimer set forth in SEQ ID NO:16 (M238);
(xiv) a homodimer set forth in SEQ ID NO:17 (M239);
(xv) a homodimer set forth in SEQ ID NO:18 (M240);
(xvi) a homodimer set forth in SEQ ID NO:19 (M241);
(xvii) a homodimer set forth in SEQ ID NO:20 (M243);
(xviii) a homodimer set forth in SEQ ID NO:21 (M244);
(xix) a homodimer set forth in SEQ ID NO:22 (M245);
(xx) a homodimer set forth in SEQ ID NO:23 (M246);
(xxi) a homodimer set forth in SEQ ID NO:24 (M247);
(xxii) a homodimer set forth in SEQ ID NO:25 (M248);
(xxiii) a homodimer set forth in SEQ ID NO:26 (M249);
(xxiv) a homodimer set forth in SEQ ID NO:27 (M327);
(xxv) a homodimer set forth in SEQ ID NO:28 (M328);
(xxvi) a homodimer set forth in SEQ ID NO:29 (M329);
(xxvii) a homodimer set forth in SEQ ID NO:30 (M330);
(xxviii) a homodimer set forth in SEQ ID NO:31 (M331); or
(xxix) a homodimer set forth in SEQ ID NO:32 (M332).

A bivalent, heterodimeric interleukin 15 (IL15) construct comprising from N-terminus to C-terminus:
a) an Interleukin 2 receptor beta (IL2Rb) domain linked to;
b) a first linker (L1) containing a protease activatable moiety, linked to;
c) an IL15 domain, comprising a first molecule and a second molecule comprising from N-terminus to C-terminus:
x) an IL15 receptor alpha (IL15Ra) domain; and
y) an IgG1 Fc region, wherein the heterodimeric IL15 construct comprises:
(i) a first molecule set forth in SEQ ID NO:33 (M43) and a second molecule set forth in SEQ ID NO:34 (M24);
(ii) a first molecule set forth in SEQ ID NO:35 (M61) and a second molecule set forth in SEQ ID NO:36 (M60); or
(iii) a first molecule set forth in SEQ ID NO:37 (M62) and a second molecule set forth in SEQ ID NO:38 (M60).

A bivalent, homodimeric interleukin 15 (IL15) construct comprising from N-terminus to C-terminus:
a) an Interleukin 2 receptor beta (IL2Rb) domain, linked to;
b) a first linker (L1) containing a protease activatable moiety linked to;
c) an IL15 domain, linked to;
d) a second linker (L2) linked to;
e) an IL15 receptor alpha (IL15Ra) domain; and
f) an IgG1 Fc region, wherein the bivalent IL15 construct comprises:
(i) a homodimer set forth in SEQ ID NO:40 (M148) and in SEQ ID NO:41 (M174).

A bivalent, homodimeric interleukin 15 (IL15) construct comprising from N-terminus to C-terminus:
a) an IgG1 Fc region, linked to;
b) a first linker (L1) linked to;
c) an Interleukin 2 receptor beta (IL2Rb) domain linked to;
d) a second linker (L2) containing a protease activatable moiety linked to;
e) an IL15 receptor alpha (IL15Ra) domain linked to;
f) a third linker (L3) linked to;
g) an IL15 domain; and wherein the bivalent IL15 construct comprises:
(i) a homodimer set forth in SEQ ID NO:42 (M232)
(ii) a homodimer set forth in SEQ ID NO:43 (M1001);
(iii) a homodimer set forth in SEQ ID NO:44 (M1002);
(vi) a homodimer set forth in SEQ ID NO:45 (M1003);
(v) a homodimer set forth in SEQ ID NO:46 (M1004);
(vi) a homodimer set forth in SEQ ID NO:47 (M1005); or
(vii) a homodimer set forth in SEQ ID NO:48 (M1006).

A monovalent, heterodimeric interleukin 15 (IL15) construct comprising from N-terminus to C-terminus:
a) an IL15 receptor alpha (IL15Ra) domain linked to;
b) a first linker (L1) linked to;
c) an IL15 domain linked to;
d) a second linker (L2) containing a protease activatable moiety linked to;
e) an Interleukin 2 receptor beta (IL2Rb) domain; and
f) a first IgG1 Fc region,
as a first molecule and a second molecule comprising a second IgG1 Fc region, wherein the heterodimeric IL15 construct comprises: a first molecule set forth in SEQ ID NO:49 (MK107) and a second molecule set forth in SEQ ID NO:50 (MH2).

A monovalent, heterodimeric interleukin 15 (IL15) construct comprising from N-terminus to C-terminus:
a) a first IgG1 Fc region linked to;
b) a first linker (L1) linked to;
c) an Interleukin 2 receptor beta (IL2Rb) domain linked to;
d) a second linker (L2) containing a protease activatable moiety linked to;
e) an IL15 receptor alpha (IL15Ra) domain linked to;
f) a third linker (L3) linked to;
g) an IL15 domain;
as a first molecule and a second molecule comprising a second IgG1 Fc region, wherein the monovalent, heterodimeric IL15 construct comprises:
(i) a first molecule set forth in SEQ ID NO:63 (M111) and a second molecule set forth in SEQ ID NO:64 (MH2);
(ii) a first molecule set forth in SEQ ID NO:65 (M2001) and a second molecule set forth in SEQ ID NO:52 (MH7); or
(iii) a first molecule set forth in SEQ ID NO:66 (M2002) and a second molecule set forth in SEQ ID NO:52 (MH7).

A monovalent, heterodimeric interleukin 15 (IL15) construct comprising from N-terminus to C-terminus:
a) an IL15 receptor alpha (IL15Ra) domain linked to;
b) a first linker (L1) linked to;
c) an IL15 domain linked to;
d) a second linker (L2) containing a protease activatable moiety linked to;
e) an Interleukin 2 receptor beta (IL2Rb) domain linked to;
f) a third linker (L3) linked to;
g) a first IgG1 Fc region,
as a first molecule and a second molecule comprising a second IgG1 Fc region, wherein the heterodimeric IL15 construct comprises:
(i) a first molecule set forth in SEQ ID NO:51 (MK114) and a second molecule set forth in SEQ ID NO:52 (MH7);
(ii) a first molecule set forth in SEQ ID NO:53 (MK115) and a second molecule set forth in SEQ ID NO:52 (MH7);
(iii) a first molecule set forth in SEQ ID NO:54 (MK117) and a second molecule set forth in SEQ ID NO:52 (MH7);
(iv) a first molecule set forth in SEQ ID NO:55 (MK118) and a second molecule set forth in SEQ ID NO:52 (MH7);
(v) a first molecule set forth in SEQ ID NO:56 (MK119) and a second molecule set forth in SEQ ID NO:52 (MH7);
(vi) a first molecule set forth in SEQ ID NO:57 (MK120) and a second molecule set forth in SEQ ID NO:52 (MH7);
(vii) a first molecule set forth in SEQ ID NO:58 (MK121) and a second molecule set forth in SEQ ID NO:52 (MH7);
(viii) a first molecule set forth in SEQ ID NO:59 (MK123) and a second molecule set forth in SEQ ID NO:52 (MH7);
(ix) a first molecule set forth in SEQ ID NO:60 (MK124) and a second molecule set forth in SEQ ID NO:52 (MH7);
(x) a first molecule set forth in SEQ ID NO:61 (MK125) and a second molecule set forth in SEQ ID NO:52 (MH7);
(xi) a first molecule set forth in SEQ ID NO:62 (MK126) and a second molecule set forth in SEQ ID NO:52 (MH7);
(xii) a first molecule set forth in SEQ ID NO:67 (MK136) and a second molecule set forth in SEQ ID NO:52 (MH7);
(xiii) a first molecule set forth in SEQ ID NO:68 (MK137) and a second molecule set forth in SEQ ID NO:52 (MH7);
(xiv) a first molecule set forth in SEQ ID NO:69 (MK138) and a second molecule set forth in SEQ ID NO:52 (MH7);
(xv) a first molecule set forth in SEQ ID NO:70 (MK139) and a second molecule set forth in SEQ ID NO:52 (MH7);
(xvi) a first molecule set forth in SEQ ID NO:71 (MK140) and a second molecule set forth in SEQ ID NO:52 (MH7);
(xvii) a first molecule set forth in SEQ ID NO:72 (MK141) and a second molecule set forth in SEQ ID NO:52 (MH7);
(xviii) a first molecule set forth in SEQ ID NO:73 (MK146) and a second molecule set forth in SEQ ID NO:52 (MH7);
(xix) a first molecule set forth in SEQ ID NO:74 (MK145) and a second molecule set forth in SEQ ID NO:75 (MH8);
(xx) a first molecule set forth in SEQ ID NO:76 (MK149) and a second molecule set forth in SEQ ID NO:75 (MH8);
(xxi) a first molecule set forth in SEQ ID NO:77 (MK150) and a second molecule set forth in SEQ ID NO:75 (MH8);
(xxii) a first molecule set forth in SEQ ID NO:78 (MK151) and a second molecule set forth in SEQ ID NO:75 (MH8);
(xxiii) a first molecule set forth in SEQ ID NO:79 (MK152) and a second molecule set forth in SEQ ID NO:75 (MH8);
(xxiv) a first molecule set forth in SEQ ID NO:80 (MK153) and a second molecule set forth in SEQ ID NO:75 (MH8);
(xxv) a first molecule set forth in SEQ ID NO:81 (MK154) and a second molecule set forth in SEQ ID NO:75 (MH8);
(xxvi) a first molecule set forth in SEQ ID NO:82 (MK155) and a second molecule set forth in SEQ ID NO:52 (MH7);
(xxvii) a first molecule set forth in SEQ ID NO:172 (MK157) and a second molecule set forth in SEQ ID NO:75 (MH8).

A monovalent, heterodimeric interleukin 15 (IL15) construct comprising from N-terminus to C-terminus:
a) a first IgG1 Fc region linked to;
b) a first linker (L1) containing a protease activatable moiety linked to;
c) an IL15 receptor alpha (IL15Ra) domain linked to;
d) a second linker (L2) linked to;
e) an IL15 domain;
as a first molecule and a second molecule comprising:
x) a second IgG1 Fc region;
y) a linker (L3) linked to;
z) an Interleukin 2 receptor beta (IL2Rb) domain; wherein the monovalent, heterodimeric IL15 construct comprises:
(i) a heterodimer set forth in SEQ ID NO:83 (M109) and set forth in SEQ ID NO:84 (MH110);
(ii) a heterodimer set forth in SEQ ID NO:85 (M2003) and set forth in SEQ ID NO:86 (MH2004); or
(iii) a heterodimer set forth in SEQ ID NO:87 (M2003) and set forth in SEQ ID NO:88 (MH2005).

A monovalent, heterodimeric interleukin 15 (IL15) construct comprising from N-terminus to C-terminus:
a) a first IgG1 Fc region linked to;
b) a first linker (L1) containing a protease activatable moiety linked to;
c) an IL15 domain linked to;
d) a second linker (L2) linked to;
e) an IL15 receptor alpha (IL15Ra) domain;
as a first molecule and a second molecule comprising:
x) a second IgG1 Fc region;
y) a linker (L3) linked to;
z) an Interleukin 2 receptor beta (IL2Rb) domain;
wherein the monovalent, heterodimeric IL15 construct comprises:
(i) a heterodimer set forth in SEQ ID NO:89 (M005) and set forth in SEQ ID NO:90 (MK5); or
(ii) a heterodimer set forth in SEQ ID NO:91 (M2006) and set forth in SEQ ID NO:90 (MK5).

A monovalent, heterodimeric interleukin 15 (IL15) construct comprising from N-terminus to C-terminus:
a) an IL15 receptor alpha (IL15Ra) domain; linked to;
b) a first linker (L1) linked to;
c) an IL15 domain linked to;
d) a second linker (L2) containing a protease activatable moiety linked to;
a first IgG1 Fc region;
as a first molecule and a second molecule comprising:
x) an Interleukin 2 receptor beta (IL2Rb) domain linked to;
y) a linker (L3) linked to;
z) a second IgG1 Fc region;
wherein the monovalent, heterodimeric IL15 construct comprises:
(i) a heterodimer set forth in SEQ ID NO:92 (M006) and set forth in SEQ ID NO:93 (MK6); or
(ii) a heterodimer set forth in SEQ ID NO:94 (M2007) and set forth in SEQ ID NO:93 (MK6).

A monovalent, heterodimeric interleukin 15 (IL15) construct comprising from N-terminus to C-terminus:
a) an Interleukin 2 receptor beta (IL2Rb) domain linked to;
b) a first linker (L1) containing a protease activatable moiety linked to;
c) an IL15 domain and;
d) a first IgG1 Fc region;
as a first molecule and a second molecule comprising:
x) an IL15 receptor alpha (IL15Ra) domain linked to;
y) a linker (L3) linked to;
z) a second IgG1 Fc region;
wherein the monovalent, heterodimeric IL15 construct comprises a first molecule set forth in SEQ ID NO:95 (M108) and a second molecule set forth in SEQ ID NO:96 (MH4).

A monovalent, heterodimeric interleukin 15 (IL15) construct comprising from N-terminus to C-terminus:
a) a first IgG1 Fc region linked to;
b) a first linker (L1) linked to;
c) an IL15 domain linked to;
d) a second linker (L2) containing a protease activatable moiety linked to;
e) an Interleukin 2 receptor beta (IL2Rb) domain;
as a first molecule and a second molecule comprising:
x) a second IgG1 Fc region linked to;
y) a linker (L3) linked to;
z) an IL15 receptor alpha (IL15Ra) domain;
wherein the monovalent, heterodimeric IL15 construct comprises:
a first molecule set forth in SEQ ID NO:97 (M112) and a second molecule set forth in SEQ ID NO:98 (MK113).

A bivalent homodimeric interleukin 15 (IL15) construct comprising from N-terminus to C-terminus:
a) a tumor associated antigen (TAA) binding antibody with a first IgG1 Fc region linked to;
b) a first linker (L1) linked to;
c) an Interleukin 2 receptor beta (IL2Rb) domain linked to;
d) a second linker (L2) containing a protease activatable moiety linked to;
e) an IL15 receptor alpha (IL15Ra) domain;
f) an IL15 domain;
wherein the bivalent, homodimeric IL15 construct comprises the sequence set forth in SEQ ID NO:99 (M001) and the sequence set forth in SEQ ID NO:100 (MH333LC).

A monovalent heterodimeric interleukin 15 (IL15) construct comprising from N-terminus to C-terminus:
a) a tumor associated antigen (TAA) binding antibody with a first IgG1 Fc region linked to;
b) a first linker (L1) linked to;
c) an Interleukin 2 receptor beta (IL2Rb) domain linked to;
d) a second linker (L2) containing a protease activatable moiety linked to;
e) an IL15 receptor alpha (IL15Ra) domain linked to a third linker linked to;
f) an IL15 domain;
wherein the monovalent, heterodimeric IL15 construct comprises the sequence set forth in SEQ ID NO:101 (M002), the sequence set forth in SEQ ID NO:102 (MH2) and the sequence set forth in SEQ ID NO:100 (MH333LC).

A monovalent heterodimeric interleukin 15 (IL15) construct comprising from N-terminus to C-terminus:
a) a tumor associated antigen (TAA) binding antibody with a first IgG1 Fc region linked to;
b) a first linker (L1) containing a protease activatable moiety linked to;
c) an IL15 receptor alpha (IL15Ra) domain linked to;
d) a second linker (L2) linked to;
e) an IL15 domain;
wherein the monovalent, heterodimeric IL15 construct comprises the sequence set forth in SEQ ID NO:103 (MK3), and
x) a tumor associated antigen (TAA) binding antibody comprising a second IgG1 Fc region linked to;
y) a first linker (L3) linked to;
z) an Interleukin 2 receptor beta (IL2Rb) domain,
wherein the sequence is set forth in SEQ ID NO:104(MH3) and is set forth in SEQ ID NO:100(MH333LC).

A monovalent heterodimeric interleukin 15 (IL15) construct comprising from N-terminus to C-terminus:
a) a first tumor associated antigen (TAA) binding antibody with a first IgG1 Fc region linked to;
b) a first linker (L1) containing a protease activatable moiety linked to;
c) an IL15 domain linked to;
d) a second linker (L2) linked to;
e) an IL15 receptor alpha (IL15Ra) domain,
wherein the monovalent, heterodimeric IL15 construct comprises the sequence set forth in SEQ ID NO:105 (MK4), and
x) a first tumor associated antigen (TAA) binding antibody with a second IgG1 Fc region linked to;
y) a first linker (L3) set forth in;
z) an Interleukin 2 receptor beta (IL2Rb) domain,
wherein the sequence is set forth in SEQ ID NO:106 (MH3) and is set forth in SEQ ID NO:100(MH333LC).

A monovalent, heterodimeric interleukin 15 (IL15) construct comprising from N-terminus to C-terminus:
a) an Interleukin 2 receptor beta (IL2Rb) domain linked to;
b) a first linker (L1) containing a protease activatable moiety linked to;
c) an IL15 domain;
as a first molecule; and a second molecule comprising
x) an IL15 receptor alpha (IL15Ra) domain; and
y) a first IgG1 Fc region,
and a third molecule comprising a second IgG1 Fc region, wherein the heterodimeric IL15 construct comprises:
a first molecule set forth in SEQ ID NO:107 (MK143), a second molecule set forth in SEQ ID NO:108 (MK144) and a third molecule set forth in SEQ ID NO:52 (H7).

A monovalent, heterodimeric interleukin 15 (IL15) construct comprising from N-terminus to C-terminus:
a) a first IgG1 Fc region linked to;
b) a first linker (L1) linked to;
c) an IL15 receptor alpha (IL15Ra) domain linked to;
d) a second linker (L2) linked to;
e) an IL15 domain
f) a third linker (L3) containing a protease activatable moiety linked to;
g) an Interleukin 2 receptor beta (IL2Rb) domain;
as a first molecule and a second molecule comprising a second IgG1 Fc region, wherein the monovalent, heterodimeric IL15 construct comprises:
   (i) a first molecule set forth in SEQ ID NO:109 (MK142) and a second molecule set forth in SEQ ID NO:52 (MH7);
   (ii) a first molecule set forth in SEQ ID NO:173 (MK156) and a second molecule set forth in SEQ ID NO:75 (MH8); or
   (iii) a first molecule set forth in SEQ ID NO:174 (MK165) and a second molecule set forth in SEQ ID NO:75 (MH8).

A monovalent, heterodimeric interleukin 15 (IL15) construct comprising from N-terminus to C-terminus:
a) an IL15 receptor alpha (IL15Ra) domain as a first molecule that is linked via a disulfide bond to;
b) an IL15 domain linked to;
c) a first linker (L1) containing a protease activatable moiety linked to;
d) an Interleukin 2 receptor beta (IL2Rb) domain linked to;
e) second linker (L2), linked to
f) a first IgG1 Fc region,
and a third molecule comprising a second IgG1 Fc region, wherein the heterodimeric IL15 construct comprises: a first molecule set forth in SEQ ID NO:110 (MK147) and a second molecule set forth in SEQ ID NO:111 (MK148) and a third molecule set forth in SEQ ID NO:52 (MH7).

A monovalent heterodimeric interleukin 15 (IL15) construct comprising from N-terminus to C-terminus:
a) a tumor associated antigen (TAA) binding antibody with a first IgG1 Fc region linked to;
b) a first linker (L1) linked to;
c) an IL15 receptor alpha (IL15Ra) domain linked to;
d) a second linker (L2) linked to;
e) an IL15 domain linked to;
f) a third linker (L3) containing a protease activatable moiety linked to;
g) an Interleukin 2 receptor beta (IL2Rb) domain; and
wherein the monovalent, heterodimeric IL15 construct comprises the sequence set forth in SEQ ID NO:175(MK14), the sequence set forth in SEQ ID NO:102(MH2) and the sequence set forth in SEQ ID NO:100(MH333LC).

A pharmaceutical composition comprising the IL15 construct of in combination with at least one additional IL15 construct.

A method of treating cancer comprising administering to a patient in need an effective amount of the IL15 construct

The method, wherein the cancer is gastric cancer, colon cancer, pancreatic cancer, breast cancer, head and neck cancer, kidney cancer, liver cancer, small cell lung cancer, non-small cell lung cancer, ovarian cancer, skin cancer, mesothelioma, lymphoma, leukemia, myeloma and sarcoma.

The method, wherein the IL15 construct is administered in combination with another therapeutic agent.

The method, wherein the therapeutic agent is an immune checkpoint agent.

The method, wherein the immune checkpoint agent is a PD-1, PD-L1, PD-L2, TIM3, LAG-3, OX40 or TIGIT antibody.

A method of increasing the survival of an immune cell, comprising administering an IL15 construct prior to, during or after administration of an effective amount of immune cells to a patient.

The method wherein the immune cell expresses a chimeric antigen receptor (CAR).

The method wherein the immune cell is an NK cell.

The method wherein the immune cell is a T-cell.

### BRIEF DESCRIPTION OF THE DRAWINGS

FIG. 1 shows a bivalent IL15 construct A.

FIG. 2 shows a bivalent IL15 construct B.

FIG. 3 shows a bivalent IL15 construct C.

FIG. 4 shows a bivalent IL15 construct D.

FIG. 5 shows the monovalent constructs E1 and E2.

FIG. 6 shows the monovalent construct E3.

FIG. 7 shows the monovalent constructs F1, F2 and F3.

FIG. 8 shows the monovalent constructs G1 and G2.

FIG. 9 shows the bivalent construct H1 and monovalent construct H2.

FIG. 10 shows the monovalent constructs K1 and K2.

FIG. 11 shows the monovalent construct M.

FIG. 12 shows the monovalent construct N.

FIG. 13 shows the monovalent construct P.

FIG. 14, shows the monovalent construct Q.

FIGS. 15-25 show the results of a cell based pSTAT5 activation assay.

FIG. 26 demonstrates that IL15 constructs have activity in a cell proliferation assay.

FIG. 27A-C shows a graphical dosing scheme for the maximum tolerated dose of IL15 constructs (FIG. 27A), the survival curve of the mice treated with IL15 constructs (FIG. 27B) and the body weight change of the mice (FIG. 27C).

FIG. 28A-B show that at the maximum tolerated dose level, Cmax and exposure of MK137/MH7 were 53 and 98 fold higher than P22339 in ICR mice in terms of relevant IL-15 concentration.

FIG. 29A-B demonstrates the dose-dependent pharmacodynamics effects of MK137/MH7 on peripheral blood cells and tumor infiltrating lymphocytes (TILS).

FIGS. 30 and 31 show the PD/PK characteristics of MK137/MH7 in an HT29/HH xenograft mouse model, wherein MK137/MH7 demonstrates a greater therapeutic window.

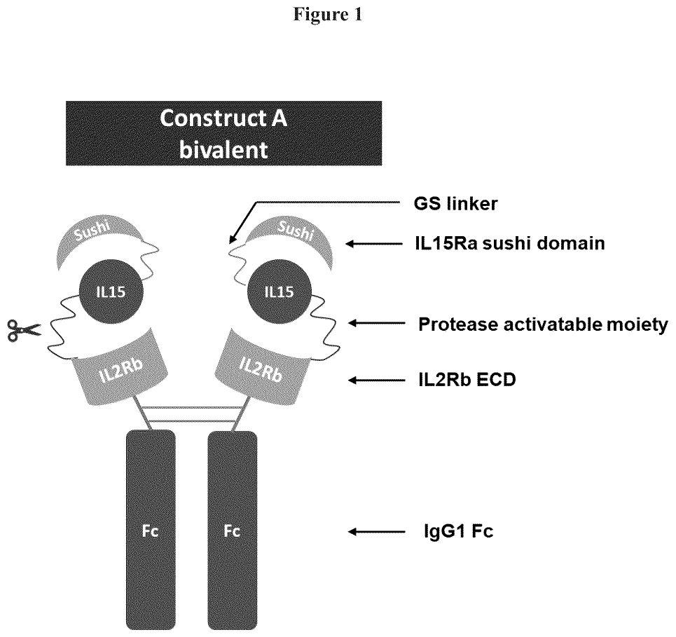

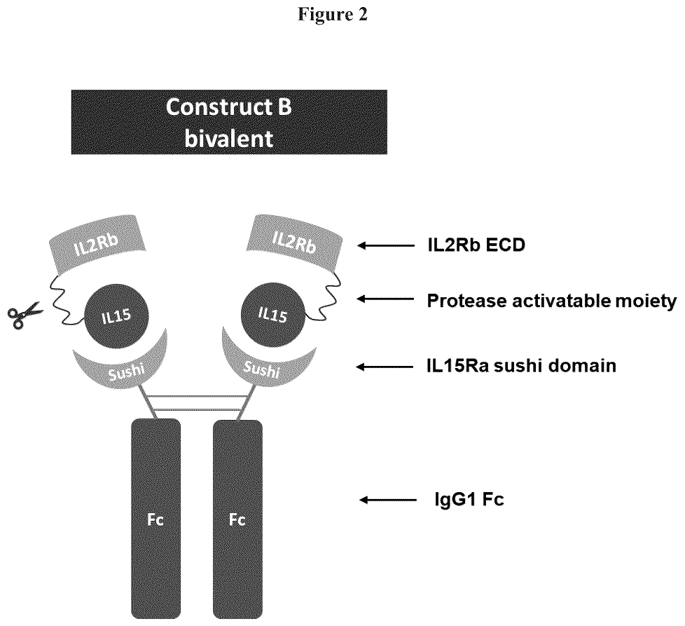

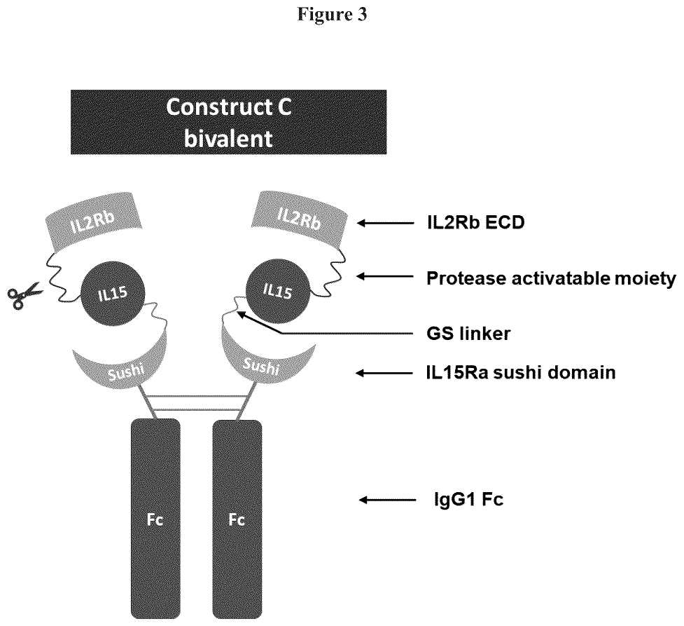

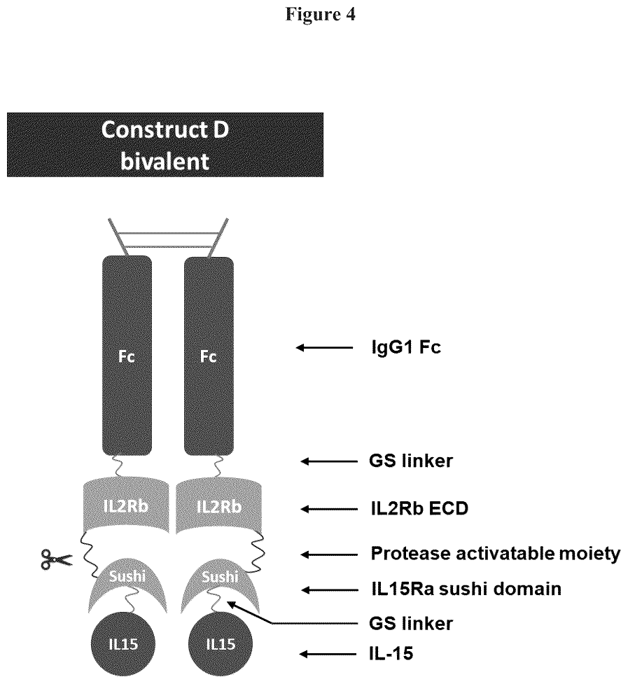

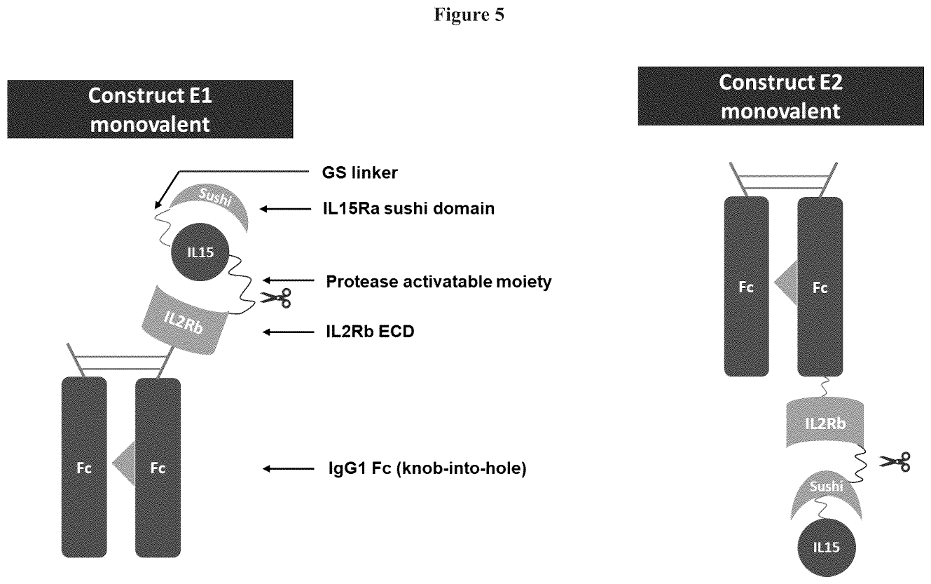

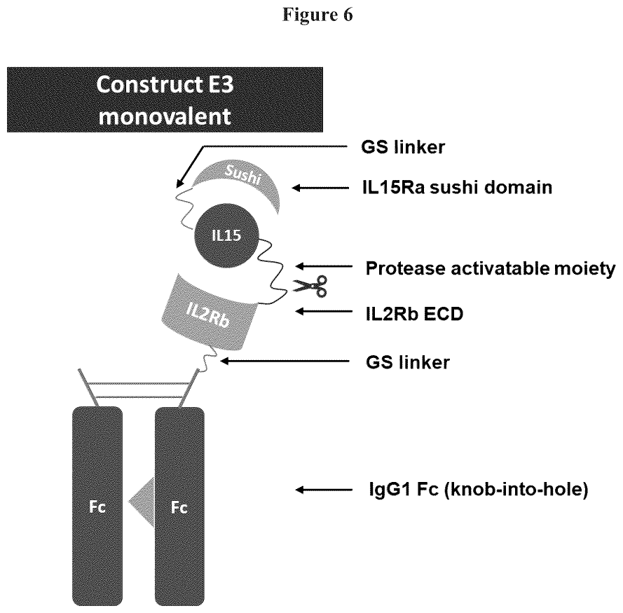

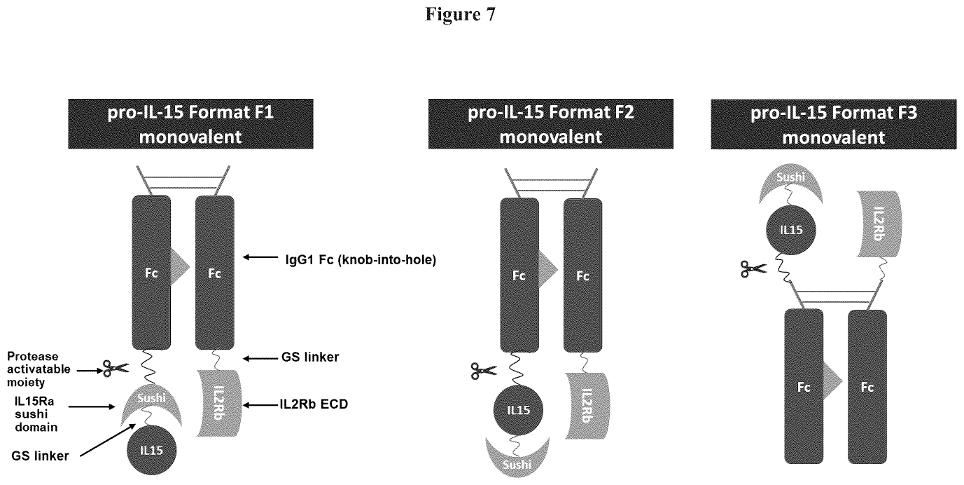

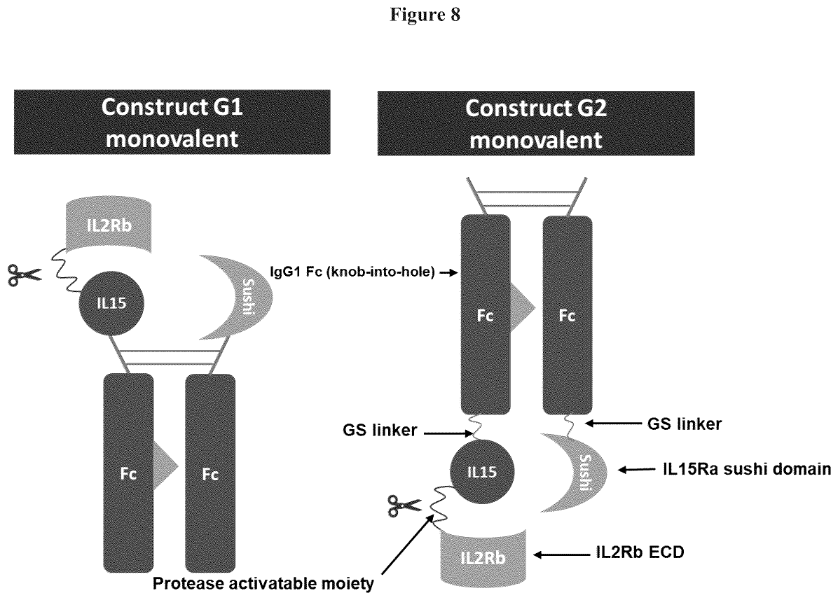

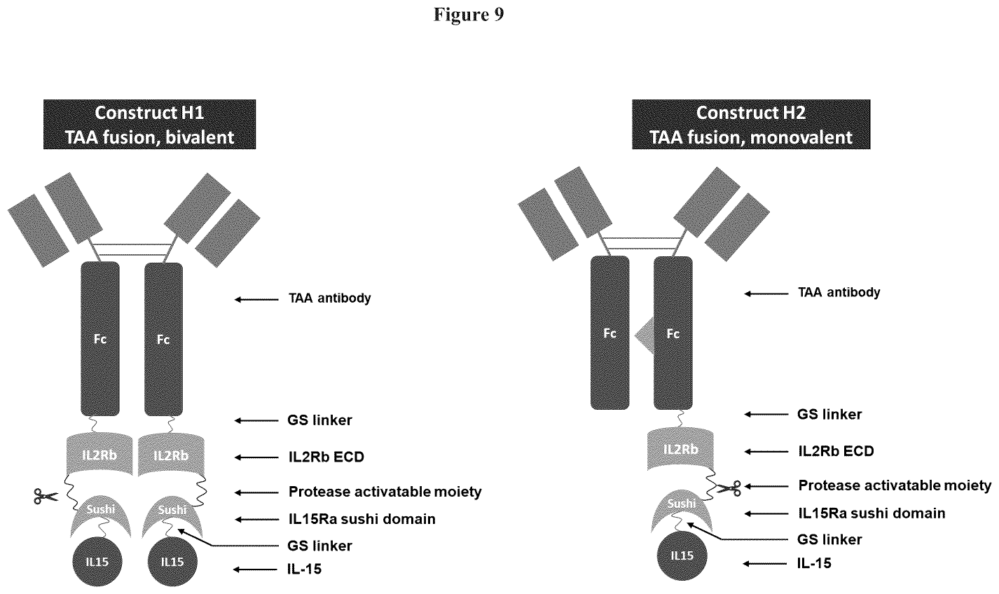

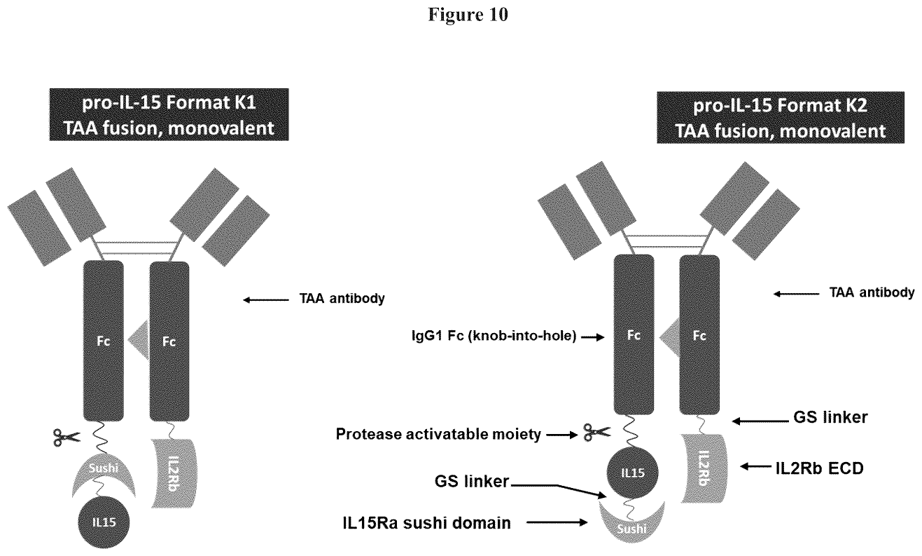

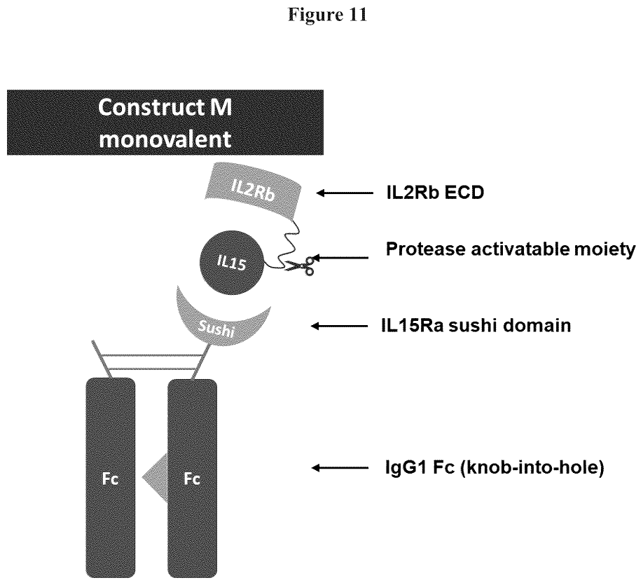

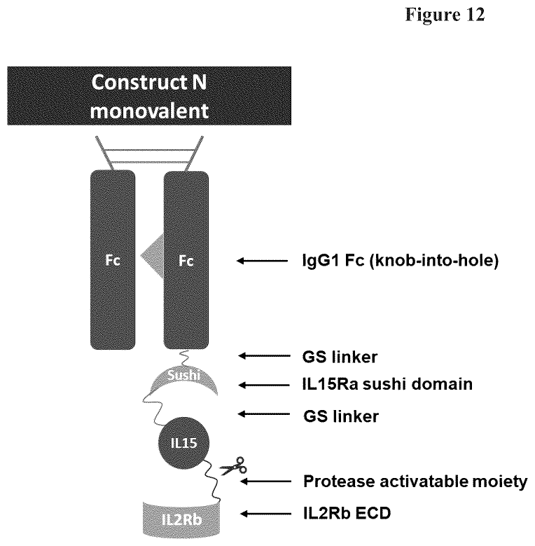

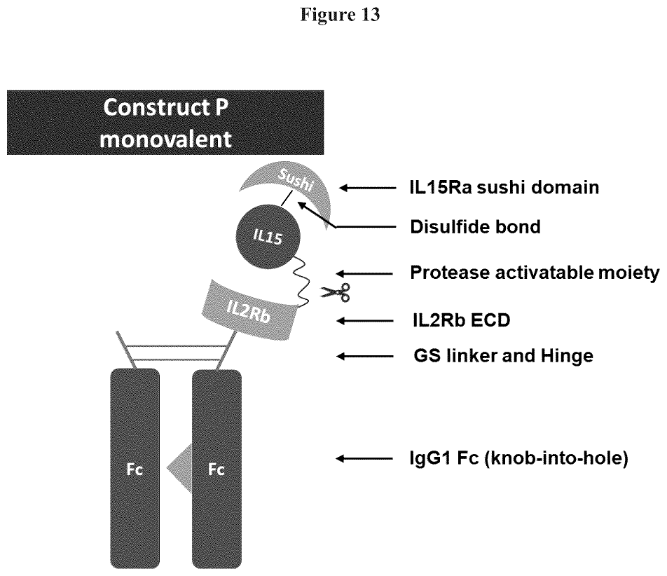

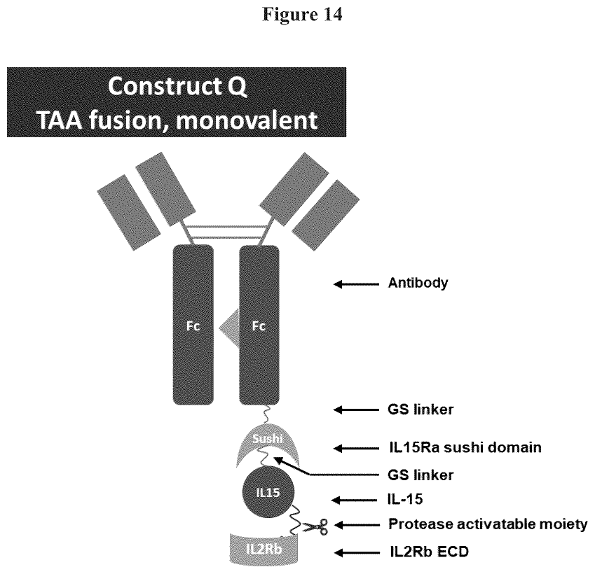

### DEFINITIONS

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

The term “Interleukin-15” or “IL15” is a cytokine that stimulates the proliferation of T-lymphocytes. The amino acid sequence of human IL15, (SEQ ID NO:1) can also be found at accession number X94223.

SEQ ID NO: 1
MRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISL
ESGDASIHDTVENLIILANNSLSSNGNVTESGCKECEELEEKNIKEFLQS
FVHIVQMFINTS

The term “Interleukin-15 receptor alpha” or “IL15Ra” is the high affinity receptor for IL15. The amino acid sequence of IL15Ra, (SEQ ID NO: 2) can also be found at accession number CR542023.

SEQ ID NO: 2
MAPRRARGCRTLGLPALLLLLLLRPPATRGITCPPPMSVEHADIWVKSYS
LYSRERYICNSGFKRKAGTSSLTECVLNKATNVAHWTTPSLKCIRDPALV
HQRPAPPSTVTTAGVTPQPESLSPSGKEPAASSPSSNNTAATTAAIVPGS
QLMPSKSPSTGTTEISSHESSHGTPSQTTAKNWELTASASHQPPGVYPQG
HSDTTVAISTSTVLLCGLSAVSLLACYLKSRQTPPLASVEMEAMEALPVT
WGTSSRDEDLENCSHHL

The term “Interleukin-2 receptor beta” or “IL2Rb” is a beta subunit receptor involved in receptor mediated endocytosis and transduces the mitogenic signals of IL2. It also associates with IL15Ra, involved in the stimulation of neutrophil phagocytosis by IL15. The amino acid sequence of human IL2Rb, (SEQ ID NO: 3) can also be found at accession number CR456506.

SEQ ID NO: 3
MAAPALSWRLPLLILLLPLATSWASAAVNGTSQFTCFYNSRANISCVWSQ
DGALQDTSCQVHAWPDRRRWNQTCELLPVSQASWACNLILGAPDSQKLTT
VDIVTLRVLCREGVRWRVMAIQDFKPFENLRLMAPISLQVVHVETHRCNI
SWEISQASHYFERHLEFEARTLSPGHTWEEAPLLTLKQKQEWICLETLTP
DTQYEFQVRVKPLQGEFTTWSPWSQPLAFRTKPAALGKDTIPWLGHLLVG
LSGAFGFIILVYLLINCRNTGPWLKKVLKCNTPDPSKFFSQLSSEHGGDV
QKWLSSPFPSSSFSPGGLAPEISPLEVLERDKVTQLLLQQDKVPEPASLS
SNHSLTSCFTNQGYFFFHLPDALEIEACQVYFTYDPYSEEDPDEGVAGAP
TGSSPQPLQPLSGEDDAYCTFPSRDDLLLFSPSLLGGPSPPSTAPGGSGA
GEERMPPSLQERVPRDWDPQPLGPPTPGVPDLVDFQPPPELVLREAGEEV
PDAGPREGVSFPWSRPPGQGEFRALNARLPLNTDAYLSLQELQGQDPTHL
V

The terms “administration,” “administering,” “treating,” and “treatment” as used herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder.

The term “subject” in the context of the present disclosure is a mammal, e.g., a primate, preferably a higher primate, e.g., a human (e.g., a patient having, or at risk of having, a disorder described herein).

The terms “cancer” or “tumor” herein has the broadest meaning as understood in the art and refers to the physiological condition in mammals that is typically characterized by unregulated cell growth. In the context of the present disclosure, the cancer is not limited to certain type or location.

The term “tumor associated antigen (TAA)” is an antigen expressed on a target tumor, wherein an antibody or antigen binding fragment of an antibody is directed to and specifically binds that TAA. For example, the TAA described herein is PD-L1 and the antibody that has been raised against this antigen is disclosed in WO 2016/000619.

In the context of the present disclosure, when reference is made to an amino acid sequence, the term “conservative substitution” means substitution of the original amino acid by anew amino acid that does not substantially alter the chemical, physical and/or functional properties of the IL15 construct, e.g. its ability to bind and activate the IL15 signaling pathway. Specifically, common conservative substations of amino acids are well known in the art and are shown below.

TABLE 1
Exemplary Amino Acid Substitutions

| Original amino | One-letter and three-letter | Conservative |
|---|---|---|
| acid residue | codes | substitution |
| Alanine | A or Ala | Gly; Ser |
| Arginine | R or Arg | Lys; His |
| Asparagine | N or Asn | Gln; His; Asp; Lys; Arg; Gln |
| Aspartic acid | D or Asp | Gln; Asn |
| Cysteine | C or Cys | Ser; Ala; Thr |
| Glutamine | Q or Gln | Asn |
| Glutamic acid | E or Glu | Asp; Gln |
| Glycine | G or Gly | Ala |
| Histidine | H or His | Asn; Gln |
| Isoleucine | I or Ile | Leu; Val |
| Leucine | L or Leu | Ile; val; Ala; Cys |
| Lysine | K or Lys | Arg; His |
| Methionine | M or Met | Leu; Ile; Tyr |
| Phenylalanine | F or Phe | Tyr; Met; Leu |
| Proline | P or Pro | Ala |
| Serine | S or Ser | Thr; Cys |
| Threonine | T or Thr | Ser; Trp |
| Tryptophan | W or Trp | Tyr; Phe |
| Tyrosine | Y or Tyr | Trp; Phe; Val; Cys |
| Valine | V or Val | Ile; Leu; Gln |

The term “knob-into-hole” technology as used herein refers to amino acids that direct the pairing of two polypeptides together either in vitro or in vivo by introducing a spatial protuberance (knob) into one polypeptide and a socket or cavity (hole) into the other polypeptide at an interface in which they interact. For example, knob-into-holes have been introduced in the Fc:Fc binding interfaces, CL:CHI interfaces or VH/VL interfaces of antibodies (see, e.g., US 2011/0287009, US2007/0178552, WO 96/027011, WO 98/050431, and Zhu et al, 1997, Protein Science 6:781-788). In some embodiments, knob-into-holes insure the correct pairing of two different heavy chains together during the expression of specific IL15 constructs. For example, IL15 constructs having knob-into-hole amino acids in their Fc regions can further comprise a first molecule of an IL15 construct and a second molecule of an IL15 construct, wherein these two molecules are assembled at least in part, through knob into hole interaction.

The term “knob” as used herein in the context of “knob-into-hole” technology refers to an amino acid change that introduces a protuberance (knob) into a polypeptide at an interface in which the polypeptide interacts with another polypeptide. In some embodiments, the other polypeptide has a hole mutation.

The term “hole” as used herein in the context of “knob-into-hole” refers to an amino acid change that introduces a socket or cavity (hole) into a polypeptide at an interface in which the polypeptide interacts with another polypeptide. In some embodiments, the other polypeptide has a knob mutation.

Examples of algorithms that are suitable for determining percent sequence identity and sequence similarity are the BLAST algorithms, which are described in Altschul et al, Nuc. Acids Res. 25:3389-3402, 1977; and Altschul et al., J. Mol. Biol. 215:403410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89: 10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4: 11-17, (1988), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444-453, (1970), algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “operably linked” or in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “linker” “linked, “linked to” or “linkered” refer a polypeptide (protein) of at least two amino acids, that are inserted between two polypeptides thus joining them together. A linker can be non-cleavable or have a protease activatable (cleavable) moiety. Examples of linkers are shown below in Table 3 and Table 4.

In some aspects, the present disclosure provides compositions, e.g., pharmaceutically acceptable compositions, which include an IL15 construct described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g. by injection or infusion).

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. Typical suitable compositions are in the form of injectable or infusion solutions. One suitable mode of administration is parenteral (e.g., intravenous, subcutaneous, intraperitoneal, intramuscular). In some embodiments, the IL15 construct is administered by intravenous infusion or injection. In certain embodiments, the IL15 construct is administered by intramuscular or subcutaneous injection.

The term “therapeutically effective amount” as herein used, refers to the amount of an IL15 construct that, when administered to a subject for treating a disease, or at least one of the clinical symptoms of a disease or disorder, is sufficient to effect such treatment for the disease, disorder, or symptom. The “therapeutically effective amount” can vary with the IL15 construct, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

The term “combination therapy” refers to the administration of two or more therapeutic agents to treat a therapeutic condition or disorder described in the present disclosure. Such administration encompasses co-administration of these therapeutic agents in a substantially simultaneous manner. Such administration also encompasses co-administration in multiple, or in separate containers (e.g., capsules, powders, and liquids) for each active ingredient. Powders and/or liquids can be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

As used herein, the phrase “in combination with” means that an IL15 construct is administered to the subject at the same time as, just before, or just after administration of an additional therapeutic agent. In certain embodiments, an IL15 construct is administered as a co-formulation with an additional therapeutic agent.

### DETAILED DESCRIPTION

The present disclosure provides for IL15 constructs that bind and activate the IL15 signaling pathway. Furthermore, the present disclosure provides IL15 constructs that have desirable pharmacokinetic characteristics and other desirable attributes, and thus can be used for reducing the likelihood of or treating cancer. The present disclosure further provides pharmaceutical compositions comprising IL15 constructs and methods of making and using such pharmaceutical compositions of IL15 constructs for the prevention and treatment of cancer and associated disorders.

Other IL15 constructs of the present disclosure include those where the amino acids or nucleic acids encoding the amino acids have been changed; yet have at least 60%, 70%, 80%, 90%, 95% or 99% percent identity to the sequences described in Table 2. In some aspects, it includes changes in the amino acid sequences wherein no more than 1, 2, 3, 4 or 5 amino acids have been changed when compared with sequences described in Table 2, while retaining substantially the same therapeutic activity.

TABLE 2

| Construct | SEQ ID NO | ID | SEQUENCE |
|---|---|---|---|
| Construct A | SEQ ID | M123 | ITCPPPMSVEHADIWVKSYSLYSRERYICNSG |
| | NO: 4 | | FKRKAGTSSLTECVLNKATNVAHWTTPSLKC |
| | | | IRSGGSGGGGSGGGSGGGGSLQNWVNVISD |
| | | | LKKIEDLIQSMHIDATLYTESDVHPSCKVTAM |
| | | | KCFLLELQVISLESGDASIHDTVENLIILANDS |
| | | | LSSNGNVTESGCKECEELEEKNIKEFLQSFVH |
| | | | IVQMFINTSGGGSISSGLLSGRSDNHGGGSSG |
| | | | GSAVNGTSQFTCFYNSRANISCVWSQDGALQ |
| | | | DTSCQVHAWPDRRRWNQTCELLPVSQASWA |
| | | | CNLILGAPDSQKLTTVDIVTLRVLCREGVRW |
| | | | RVMAIQDFKPFENLRLMAPISLQVVHVETHR |
| | | | CNISWEISQASHYFERHLEFEARTLSPGHTWE |
| | | | EAPLLTLKQKQEWICLETLTPDTQYEFQVRV |
| | | | KPLQGEFTTWSPWSQPLAFRTKPAALGKDEP |
| | | | KSCDKTHTCPPCPAPELLGGPSVFLFPPKPKD |
| | | | TLMISRTPEVTCVVVDVSHEDPEVKFNWYV |
| | | | DGVEVHNAKTKPREEQYNSTYRVVSVLTVL |
| | | | HQDWLNGKEYKCKVSNKALPAPIEKTISKAK |
| | | | GQPREPQVYTLPPSRDELTKNQVSLTCLVKG |
| | | | FYPSDIAVEWESNGQPENNYKTTPPVLDSDG |
| | | | SFFLYSKLTVDKSRWQQGNVFSCSVMHEAL |
| | | | HNHYTQKSLSLSPGK |
| | SEQ ID | M135 | ITCPPPMSVEHADIWVKSYSLYSRERYICNSG |
| | NO: 5 | | FKRKAGTSSLTECVLNKATNVAHWTTPSLKC |
| | | | IRSGGSGGGGSGGGSGGGGSLQNWVNVISD |
| | | | LKKIEDLIQSMHIDATLYTESDVHPSCKVTAM |
| | | | KCFLLELQVISLESGDASIHDTVENLIILANDS |
| | | | LSSNGNVTESGCKECEELEEKNIKEFLQSFVH |
| | | | IVQMFINTSGGGSIPVSLRSGGGGSSGGSGGS |
| | | | GGAVNGTSQFTCFYNSRANISCVWSQDGAL |
| | | | QDTSCQVHAWPDRRRWNQTCELLPVSQAS |
| | | | WACNLILGAPDSQKLTTVDIVTLRVLCREGV |
| | | | RWRVMAIQDFKPFENLRLMAPISLQVVHVET |
| | | | HRCNISWEISQASHYFERHLEFEARTLSPGHT |
| | | | WEEAPLLTLKQKQEWICLETLTPDTQYEFQV |
| | | | RVKPLQGEFTTWSPWSQPLAFRTKPAALGKD |
| | | | EPKSCDKTHTCPPCPAPELLGGPSVFLFPPKP |
| | | | KDTLMISRTPEVTCVVVDVSHEDPEVKFNW |
| | | | YVDGVEVHNAKTKPREEQYNSTYRVVSVLT |
| | | | VLHQDWLNGKEYKCKVSNKALPAPIEKTISK |
| | | | AKGQPREPQVYTLPPSRDELTKNQVSLTCLV |
| | | | KGFYPSDIAVEWESNGQPENNYKTTPPVLDS |
| | | | DGSFFLYSKLTVDKSRWQQGNVFSCSVMHE |
| | | | ALHNHYTQKSLSLSPGK |
| | SEQ ID | M140 | ITCPPPMSVEHADIWVKSYSLYSRERYICNSG |
| | NO: 6 | | FKRKAGTSSLTECVLNKATNVAHWTTPSLKC |
| | | | IRSGGSGGGGSGGGSGGGGSLQNWVNVISD |
| | | | LKKIEDLIQSMHIDATLYTESDVHPSCKVTAM |
| | | | KCFLLELQVISLESGDASIHDTVENLIILANDS |
| | | | LSSNGNVTESGCKECEELEEKNIKEFLQSFVH |
| | | | IVQMFINTSGGGSSGGSGGSGGSGGGSGGGG |
| | | | SGAVNGTSQFTCFYNSRANISCVWSQDGALQ |
| | | | DTSCQVHAWPDRRRWNQTCELLPVSQASWA |
| | | | CNLILGAPDSQKLTTVDIVTLRVLCREGVRW |
| | | | RVMAIQDFKPFENLRLMAPISLQVVHVETHR |
| | | | CNISWEISQASHYFERHLEFEARTLSPGHTWE |
| | | | EAPLLTLKQKQEWICLETLTPDTQYEFQVRV |
| | | | KPLQGEFTTWSPWSQPLAFRTKPAALGKDEP |
| | | | KSCDKTHTCPPCPAPELLGGPSVFLFPPKPKD |
| | | | TLMISRTPEVTCVVVDVSHEDPEVKFNWYV |
| | | | DGVEVHNAKTKPREEQYNSTYRVVSVLTVL |
| | | | HQDWLNGKEYKCKVSNKALPAPIEKTISKAK |
| | | | GQPREPQVYTLPPSRDELTKNQVSLTCLVKG |
| | | | FYPSDIAVEWESNGQPENNYKTTPPVLDSDG |
| | | | SFFLYSKLTVDKSRWQQGNVFSCSVMHEAL |
| | | | HNHYTQKSLSLSPGK |
| | SEQ ID | M145 | ITCPPPMSVEHADIWVKSYSLYSRERYICNSG |
| | NO: 7 | | FKRKAGTSSLTECVLNKATNVAHWTTPSLKC |
| | | | IRSGGSGGGGSGGGSGGGGSLQNWVNVISD |
| | | | LKKIEDLIQSMHIDATLYTESDVHPSCKVTAM |
| | | | KCFLLELQVISLESGDASIHDTVENLIILANDS |
| | | | LSSNGNVTESGCKECEELEEKNIKEFLQSFVH |
| | | | IVQMFINTSGGGSVPLSLYSGGGGSSGGSGGS |
| | | | GGAVNGTSQFTCFYNSRANISCVWSQDGAL |
| | | | QDTSCQVHAWPDRRRWNQTCELLPVSQAS |
| | | | WACNLILGAPDSQKLTTVDIVTLRVLCREGV |
| | | | RWRVMAIQDFKPFENLRLMAPISLQVVHVET |
| | | | HRCNISWEISQASHYFERHLEFEARTLSPGHT |
| | | | WEEAPLLTLKQKQEWICLETLTPDTQYEFQV |
| | | | RVKPLQGEFTTWSPWSQPLAFRTKPAALGKD |
| | | | EPKSCDKTHTCPPCPAPELLGGPSVFLFPPKP |
| | | | KDTLMISRTPEVTCVVVDVSHEDPEVKFNW |
| | | | YVDGVEVHNAKTKPREEQYNSTYRVVSVLT |
| | | | VLHQDWLNGKEYKCKVSNKALPAPIEKTISK |
| | | | AKGQPREPQVYTLPPSRDELTKNQVSLTCLV |
| | | | KGFYPSDIAVEWESNGQPENNYKTTPPVLDS |
| | | | DGSFFLYSKLTVDKSRWQQGNVFSCSVMHE |
| | | | ALHNHYTQKSLSLSPGK |
| | SEQ ID | M175 | ITCPPPMSVEHADIWVKSYSLYSRERYICNSG |
| | NO: 8 | | FKRKAGTSSLTECVLNKATNVAHWTTPSLKC |
| | | | IRSGGSGGGGSGGGSGGGGSLQNWVNVISD |
| | | | LKKIEDLIQSMHIDATLYTESDVHPSCKVTAM |
| | | | KCFLLELQVISLESGDASIHDTVENLIILANNS |
| | | | LSSNGNVTESGCKECEELEEKNIKEFLQSFVH |
| | | | IVQMFINTSGGGSISSGLLSGRSDNHGGGSSG |
| | | | GSAVNGTSQFTCFYNSRANISCVWSQDGALQ |
| | | | DTSCQVHAWPDRRRWNQTCELLPVSQASWA |
| | | | CNLILGAPDSQKLTTVDIVTLRVLCREGVRW |
| | | | RVMAIQDFKPFENLRLMAPISLQVVHVETHR |
| | | | CNISWEISQASHYFERHLEFEARTLSPGHTWE |
| | | | EAPLLTLKQKQEWICLETLTPDTQYEFQVRV |
| | | | KPLQGEFTTWSPWSQPLAFRTKPAALGKDEP |
| | | | KSCDKTHTCPPCPAPELLGGPSVFLFPPKPKD |
| | | | TLMISRTPEVTCVVVDVSHEDPEVKFNWYV |
| | | | DGVEVHNAKTKPREEQYNSTYRVVSVLTVL |
| | | | HQDWLNGKEYKCKVSNKALPAPIEKTISKAK |
| | | | GQPREPQVYTLPPSRDELTKNQVSLTCLVKG |
| | | | FYPSDIAVEWESNGQPENNYKTTPPVLDSDG |
| | | | SFFLYSKLTVDKSRWQQGNVFSCSVMHEAL |
| | | | HNHYTQKSLSLSPGK |
| | SEQ ID | M176 | ITCPPPMSVEHADIWVKSYSLYSRERYICNSG |
| | NO: 9 | | FKRKAGTSSLTECVLNKATNVAHWTTPSLKC |
| | | | IRSGGSGGGGSGGGSGGGGSLQNWVNVISD |
| | | | LKKIEDLIQSMHIDATLYTESDVHPSCKVTAM |
| | | | KCFLLELQVISLESGDASIHDTVENLIILANNS |
| | | | LSSNGNVTESGCKECEELEEKNIKEFLQSFVH |
| | | | IVQMFINTSGGGSIPVSLRSGGGGSSGGSGGS |
| | | | GGAVNGTSQFTCFYNSRANISCVWSQDGAL |
| | | | QDTSCQVHAWPDRRRWNQTCELLPVSQAS |
| | | | WACNLILGAPDSQKLTTVDIVTLRVLCREGV |
| | | | RWRVMAIQDFKPFENLRLMAPISLQVVHVET |
| | | | HRCNISWEISQASHYFERHLEFEARTLSPGHT |
| | | | WEEAPLLTLKQKQEWICLETLTPDTQYEFQV |
| | | | RVKPLQGEFTTWSPWSQPLAFRTKPAALGKD |
| | | | EPKSCDKTHTCPPCPAPELLGGPSVFLFPPKP |
| | | | KDTLMISRTPEVTCVVVDVSHEDPEVKFNW |
| | | | YVDGVEVHNAKTKPREEQYNSTYRVVSVLT |
| | | | VLHQDWLNGKEYKCKVSNKALPAPIEKTISK |
| | | | AKGQPREPQVYTLPPSRDELTKNQVSLTCLV |
| | | | KGFYPSDIAVEWESNGQPENNYKTTPPVLDS |
| | | | DGSFFLYSKLTVDKSRWQQGNVFSCSVMHE |
| | | | ALHNHYTQKSLSLSPGK |
| | SEQ ID | M177 | ITCPPPMSVEHADIWVKSYSLYSRERYICNSG |
| | NO: 10 | | FKRKAGTSSLTECVLNKATNVAHWTTPSLKC |
| | | | IRSGGSGGGGSGGGSGGGGSLQNWVNVISD |
| | | | LKKIEDLIQSMHIDATLYTESDVHPSCKVTAM |
| | | | KCFLLELQVISLESGDASIHDTVENLIILANDS |
| | | | LSSNGNVTESGCKECEELEEKNIKEFLQSFVH |
| | | | IVQMFINTSGGGSVPLSLYSGWRSGGSGGGG |
| | | | SGSGAVNGTSQFTCFYNSRANISCVWSQDGA |
| | | | LQDTSCQVHAWPDRRRWNQTCELLPVSQAS |
| | | | WACNLILGAPDSQKLTTVDIVTLRVLCREGV |
| | | | RWRVMAIQDFKPFENLRLMAPISLQVVHVET |
| | | | HRCNISWEISQASHYFERHLEFEARTLSPGHT |
| | | | WEEAPLLTLKQKQEWICLETLTPDTQYEFQV |
| | | | RVKPLQGEFTTWSPWSQPLAFRTKPAALGKD |
| | | | EPKSCDKTHTCPPCPAPELLGGPSVFLFPPKP |
| | | | KDTLMISRTPEVTCVVVDVSHEDPEVKFNW |
| | | | YVDGVEVHNAKTKPREEQYNSTYRVVSVLT |
| | | | VLHQDWLNGKEYKCKVSNKALPAPIEKTISK |
| | | | AKGQPREPQVYTLPPSRDELTKNQVSLTCLV |
| | | | KGFYPSDIAVEWESNGQPENNYKTTPPVLDS |
| | | | DGSFFLYSKLTVDKSRWQQGNVFSCSVMHE |
| | | | ALHNHYTQKSLSLSPGK |
| | SEQ ID | M178 | ITCPPPMSVEHADIWVKSYSLYSRERYICNSG |
| | NO: 11 | | FKRKAGTSSLTECVLNKATNVAHWTTPSLKC |
| | | | IRSGGSGGGGSGGGSGGGGSLQNWVNVISD |
| | | | LKKIEDLIQSMHIDATLYTESDVHPSCKVTAM |
| | | | KCFLLELQVISLESGDASIHDTVENLIILANDS |
| | | | LSSNGNVTESGCKECEELEEKNIKEFLQSFVH |
| | | | IVQMFINTSGGGSVPLSLYSGRSASGGSGGG |
| | | | GSGSGAVNGTSQFTCFYNSRANISCVWSQDG |
| | | | ALQDTSCQVHAWPDRRRWNQTCELLPVSQA |
| | | | SWACNLILGAPDSQKLTTVDIVTLRVLCREG |
| | | | VRWRVMAIQDFKPFENLRLMAPISLQVVHVE |
| | | | THRCNISWEISQASHYFERHLEFEARTLSPGH |
| | | | TWEEAPLLTLKQKQEWICLETLTPDTQYEFQ |
| | | | VRVKPLQGEFTTWSPWSQPLAFRTKPAALGK |
| | | | DEPKSCDKTHTCPPCPAPELLGGPSVFLFPPK |
| | | | PKDTLMISRTPEVTCVVVDVSHEDPEVKFNW |
| | | | YVDGVEVHNAKTKPREEQYNSTYRVVSVLT |
| | | | VLHQDWLNGKEYKCKVSNKALPAPIEKTISK |
| | | | AKGQPREPQVYTLPPSRDELTKNQVSLTCLV |
| | | | KGFYPSDIAVEWESNGQPENNYKTTPPVLDS |
| | | | DGSFFLYSKLTVDKSRWQQGNVFSCSVMHE |
| | | | ALHNHYTQKSLSLSPGK |
| | SEQ ID | M207 | ITCPPPMSVEHADIWVKSYSLYSRERYICNSG |
| | NO: 12 | | FKRKAGTSSLTECVLNKATNVAHWTTPSLKC |
| | | | IRSGGSGGGGSGGGSGGGGSLQNWVNVISD |
| | | | LKKIEDLIQSMHIDATLYTESDVHPSCKVTAM |
| | | | KCFLLELQVISLESGDASIHDTVENLIILANNS |
| | | | LSSNGNVTESGCKECEELEEKNIKEFLQSFVH |
| | | | IVQMFINTSGGGSIPVSLRSGGGGSSGGSGGS |
| | | | GGAVNGTSQFTCFYNSRANISCVWSQDGAL |
| | | | QDTSCQVHAWPDRRRWNQTCELLPVSQAS |
| | | | WACNLILGAPDSQKLTTVDIVTLRVLCREGV |
| | | | RWRVMAIQDFKPFENLRLMAPISLQVVHVET |
| | | | HRANISWEISQASHYFERHLEFEARTLSPGHT |
| | | | WEEAPLLTLKQKQEWISLETLTPDTQYEFQV |
| | | | RVKPLQGEFTTWSPWSQPLAFRTKPAALGKD |
| | | | EPKSSDKTHTCPPCPAPELLGGPSVFLFPPKP |
| | | | KDTLMISRTPEVTCVVVDVSHEDPEVKFNW |
| | | | YVDGVEVHNAKTKPREEQYNSTYRVVSVLT |
| | | | VLHQDWLNGKEYKCKVSNKALPAPIEKTISK |
| | | | AKGQPREPQVYTLPPSRDELTKNQVSLTCLV |
| | | | KGFYPSDIAVEWESNGQPENNYKTTPPVLDS |
| | | | DGSFFLYSKLTVDKSRWQQGNVFSCSVMHE |
| | | | ALHNHYTQKSLSLSPGK |
| | SEQ ID | M231 | ITCPPPMSVEHADIWVKSYSLYSRERYICNSG |
| | NO: 13 | | FKRKAGTSSLTECVLNKATNVAHWTTPSLKC |
| | | | IRSGGSGGGGSGGGSGGGGSLQNWVNVISD |
| | | | LKKIEDLIQSMHIDATLYTESDVHPSCKVTAM |
| | | | KCFLLELQVISLESGDASIHDTVENLIILANNS |
| | | | LSSNGNVTESGCKECEELEEKNIKEFLQSFVH |
| | | | IVQMFINTSGGGSIPVSLRSGGGGSSGGSGGS |
| | | | GGAVNGTSQFTCFYNSRANISCVWSQDGAL |
| | | | QDTSCQVHAWPDRRRWNQTCELLPVSQAS |
| | | | WACNLILGAPDSQKLTTVDIVTLRVLCREGV |
| | | | RWRVMAIQDFKPEENLRLMAPISLQVVHVET |
| | | | HRCNISWEISQASHYFERHLEFEARTLSPGHT |
| | | | WEEAPLLTLKQKQEWICLETLTPDTQYEFQV |
| | | | RVKPLQEPKSSDKTHTCPPCPAPELLGGPSVF |
| | | | LFPPKPKDTLMISRTPEVTCVVVDVSHEDPE |
| | | | VKFNWYVDGVEVHNAKTKPREEQYNSTYR |
| | | | VVSVLTVLHQDWLNGKEYKCKVSNKALPAP |
| | | | IEKTISKAKGQPREPQVYTLPPSRDELTKNQV |
| | | | SLTCLVKGFYPSDIAVEWESNGQPENNYKTT |
| | | | PPVLDSDGSFFLYSKLTVDKSRWQQGNVFSC |
| | | | SVMHEALHNHYTQKSLSLSPGK |
| | SEQ ID | M233 | ITCPPPMSVEHADIWVKSYSLYSRERYICNSG |
| | NO: 14 | | FKRKAGTSSLTECVLNKATNVAHWTTPSLKC |
| | | | IRSGGSGGGGSGGGSGGGGSLQNWVNVISD |
| | | | LKKIEDLIQSMHIDATLYTESDVHPSCKVTAM |
| | | | KCFLLELQVISLESGDASIHDTVENLIILANDS |
| | | | LSSNGNVTESGCKECEELEEKNIKEFLQSFVH |
| | | | IVQMFINTSGGGSIPVSLRSGGGGSSGGSGGS |
| | | | GGAVNGTSQFTCFYNSRANISCVWSQDGAL |
| | | | QDTSCQVHAWPDRRRWNQTCELLPVSQAS |
| | | | WACNLILGAPDSQKLTTVDIVTLRVLCREGV |
| | | | RWRVMAIQDFKPFENLRLMAPISLQVVHVET |
| | | | HRCNISWEISQASHYFERHLEFEARTLSPGHT |
| | | | WEEAPLLTLKQKQEWICLETLTPDTQYEFQV |
| | | | RVKPLQGEFTTWSPWSQPLAFRTKPAALGKD |
| | | | EPKSSDKTHTCPPCPAPELLGGPSVFLFPPKP |
| | | | KDTLMISRTPEVTCVVVDVSHEDPEVKFNW |
| | | | YVDGVEVHNAKTKPREEQYNSTYRVVSVLT |
| | | | VLHQDWLNGKEYKCKVSNKALPAPIEKTISK |
| | | | AKGQPREPQVYTLPPSRDELTKNQVSLTCLV |
| | | | KGFYPSDIAVEWESNGQPENNYKTTPPVLDS |
| | | | DGSFFLYSKLTVDKSRWQQGNVFSCSVMHE |
| | | | ALHNHYTQKSLSLSPGK |
| | SEQ ID | M234 | ITCPPPMSVEHADIWVKSYSLYSRERYICNSG |
| | NO: 15 | | FKRKAGTSSLTECVLNKATNVAHWTTPSLKC |
| | | | IRSGGSGGGGSGGGSGGGGSLQNWVNVISD |
| | | | LKKIEDLIQSMHIDATLYTESDVHPSCKVTAM |
| | | | KCFLLELQVISLESGDASIHDTVENLIILANDS |
| | | | LSSNGNVTESGCKECEELEEKNIKEFLQSFVH |
| | | | IVQMFINTSGGGSIPVSLRSGGGGSSGGSGGS |
| | | | GGAVNGTSQFTCFYNSRANISCVWSQDGAL |
| | | | QDTSCQVHAWPDRRRWNQTCELLPVSQAS |
| | | | WACNLILGAPDSQKLTTVDIVTLRVLCREGV |
| | | | RWRVMAIQDFKPFENLRLMAPISLQVVHVET |
| | | | HRCNISWEISQASHYFERHLEFEARTLSPGHT |
| | | | WEEAPLLTLKQKQEWICLETLTPDTQYEFQV |
| | | | RVKPLQGEFTTWSPWSQPLAFRTKPAALGKD |
| | | | GGGGSEPKSSDKTHTCPPCPAPELLGGPSVFL |
| | | | FPPKPKDTLMISRTPEVTCVVVDVSHEDPEV |
| | | | KFNWYVDGVEVHNAKTKPREEQYNSTYRV |
| | | | VSVLTVLHQDWLNGKEYKCKVSNKALPAPI |
| | | | EKTISKAKGQPREPQVYTLPPSRDELTKNQVS |
| | | | LTCLVKGFYPSDIAVEWESNGQPENNYKTTP |
| | | | PVLDSDGSFFLYSKLTVDKSRWQQGNVFSCS |
| | | | VMHEALHNHYTQKSLSLSPGK |
| | SEQ ID | M238 | ITCPPPMSVEHADIWVKSYSLYSRERYICNSG |
| | NO: 16 | | FKRKAGTSSLTECVLNKATNVAHWTTPSLKC |
| | | | IRSGGSGGGGSGGGSGGGGSLQNWVNVISD |
| | | | LKKIEDLIQSMHIDATLYTESDVHPSCKVTAM |
| | | | KCFLLELQVISLESGDASIHDTVENLIILANNS |
| | | | LSSNGNVTESGCKECEELEEKNIKEFLQSFVH |
| | | | IVQMFINTSGGGSIPVSLRSGGGGSSGGSGGS |
| | | | GGAVNGTSQFTCFYNSRANISCVWSQDGAL |
| | | | QDTSCQVHAWPDRRRWNQTCELLPVSQAS |
| | | | WACNLILGAPDSQKLTTVDIVTLRVLCREGV |
| | | | RWRVMAIQDFKPFENLRLMAPISLQVVHVET |
| | | | HRANISWEISQASHYFERHLEFEARTLSPGHT |
| | | | WEEAPLLTLKQKQEWISLETLTPDTQYEFQV |
| | | | RVKPLQGEFTTWSPWSQPLAFRTKPAALGKD |
| | | | GGGGSEPKSSDKTHTCPPCPAPELLGGPSVFL |
| | | | FPPKPKDTLMISRTPEVTCVVVDVSHEDPEV |
| | | | KFNWYVDGVEVHNAKTKPREEQYNSTYRV |
| | | | VSVLTVLHQDWLNGKEYKCKVSNKALPAPI |
| | | | EKTISKAKGQPREPQVYTLPPSRDELTKNQVS |
| | | | LTCLVKGFYPSDIAVEWESNGQPENNYKTTP |
| | | | PVLDSDGSFFLYSKLTVDKSRWQQGNVFSCS |
| | | | VMHEALHNHYTQKSLSLSPGK |
| | SEQ ID | M239 | ITCPPPMSVEHADIWVKSYSLYSRERYICNSG |
| | NO: 17 | | FKRKAGTSSLTECVLNKATNVAHWTTPSLKC |
| | | | IRSGGSGGGGSGGGSGGGGSLQNWVNVISD |
| | | | LKKIEDLIQSMHIDATLYTESDVHPSCKVTAM |
| | | | KCFLLELQVISLESGDASIHDTVENLIILANNS |
| | | | LSSNGNVTESGCKECEELEEKNIKEFLQSFVH |
| | | | IVQMFINTSGGGSIPVSLRSGGGGSSGGSGGS |
| | | | GGAVNGTSQFTCFYNSRANISCVWSQDGAL |
| | | | QDTSCQVHAWPDRRRWNQTCELLPVSQAS |
| | | | WACNLILGAPDSQKLTTVDIVTLRVLCREGV |
| | | | RWRVMAIQDFKPFENLRLMAPISLQVVHVET |
| | | | HRANISWEISQASHYFERHLEFEARTLSPGHT |
| | | | WEEAPLLTLKQKQEWISLETLTPDTQYEFQV |
| | | | RVKPLQGEFTTWSPWSQPLAFRTKPAALGKD |
| | | | GGGGSGGGGSEPKSSDKTHTCPPCPAPELLG |
| | | | GPSVFLFPPKPKDTLMISRTPEVTCVVVDVSH |
| | | | EDPEVKFNWYVDGVEVHNAKTKPREEQYN |
| | | | STYRVVSVLTVLHQDWLNGKEYKCKVSNKA |
| | | | LPAPIEKTISKAKGQPREPQVYTLPPSRDELTK |
| | | | NQVSLTCLVKGFYPSDIAVEWESNGQPENNY |
| | | | KTTPPVLDSDGSFFLYSKLTVDKSRWQQGNV |
| | | | FSCSVMHEALHNHYTQKSLSLSPGK |
| | SEQ ID | M240 | ITCPPPMSVEHADIWVKSYSLYSRERYICNSG |
| | NO: 18 | | FKRKAGTSSLTECVLNKATNVAHWTTPSLKC |
| | | | IRSGGSGGGGSGGGSGGGGSLQNWVNVISD |
| | | | LKKIEDLIQSMHIDATLYTESDVHPSCKVTAM |
| | | | KCFLLELQVISLESGDASIHDTVENLIILANNS |
| | | | LSSNGNVTESGCKECEELEEKNIKEFLQSFVH |
| | | | IVQMFINTSGGGSIPVSLRSGGGGSSGGSGGS |
| | | | GGAVNGTSQFTCFYNSRANISCVWSQDGAL |
| | | | QDTSCQVHAWPDRRRWNQTCELLPVSQAS |
| | | | WACNLILGAPDSQKLTTVDIVTLRVLCREGV |
| | | | RWRVMAIQDFKPEENLRLMAPISLQVVHVET |
| | | | HRCNISWEISQASHYFERHLEFEARTLSPGHT |
| | | | WEEAPLLTLKQKQEWICLETLTPDTQYEFQV |
| | | | RVKPLQGGGGSEPKSSDKTHTCPPCPAPELL |
| | | | GGPSVFLFPPKPKDTLMISRTPEVTCVVVDVS |
| | | | HEDPEVKFNWYVDGVEVHNAKTKPREEQY |
| | | | NSTYRVVSVLTVLHQDWLNGKEYKCKVSNK |
| | | | ALPAPIEKTISKAKGQPREPQVYTLPPSRDELT |
| | | | KNQVSLTCLVKGFYPSDIAVEWESNGQPENN |
| | | | YKTTPPVLDSDGSFFLYSKLTVDKSRWQQGN |
| | | | VFSCSVMHEALHNHYTQKSLSLSPGK |
| | SEQ ID | M241 | ITCPPPMSVEHADIWVKSYSLYSRERYICNSG |
| | NO: 19 | | FKRKAGTSSLTECVLNKATNVAHWTTPSLKC |
| | | | IRSGGSGGGGSGGGSGGGGSLQNWVNVISD |
| | | | LKKIEDLIQSMHIDATLYTESDVHPSCKVTAM |
| | | | KCFLLELQVISLESGDASIHDTVENLIILANNS |
| | | | LSSNGNVTESGCKECEELEEKNIKEFLQSFVH |
| | | | IVQMFINTSGGGSIPVSLRSGGGGSSGGSGGG |
| | | | AVNGTSQFTCFYNSRANISCVWSQDGALQDT |
| | | | SCQVHAWPDRRRWNQTCELLPVSQASWACN |
| | | | LILGAPDSQKLTTVDIVTLRVLCREGVRWRV |
| | | | MAIQDFKPFENLRLMAPISLQVVHVETHRCN |
| | | | ISWEISQASHYFERHLEFEARTLSPGHTWEEA |
| | | | PLLTLKQKQEWICLETLTPDTQYEFQVRVKPL |
| | | | QGEFTTWSPWSQPLAFRTKGGGGSEPKSSDK |
| | | | THTCPPCPAPELLGGPSVFLFPPKPKDTLMISR |
| | | | TPEVTCVVVDVSHEDPEVKFNWYVDGVEV |
| | | | HNAKTKPREEQYNSTYRVVSVLTVLHQDWL |
| | | | NGKEYKCKVSNKALPAPIEKTISKAKGQPRE |
| | | | PQVYTLPPSRDELTKNQVSLTCLVKGFYPSDI |
| | | | AVEWESNGQPENNYKTTPPVLDSDGSFFLYS |
| | | | KLTVDKSRWQQGNVFSCSVMHEALHNHYT |
| | | | QKSLSLSPGK |
| | SEQ ID | | ITCPPPMSVEHADIWVKSYSLYSRERYICNSG |
| | NO: 20 | M243 | FKRKAGTSSLTECVLNKATNVAHWTTPSLKC |
| | | | IRSGGSGGGGSGGGSGGGGSLQNWVNVISD |
| | | | LKKIEDLIQSMHIDATLYTESDVHPSCKVTAM |
| | | | KCFLLELQVISLESGDASIHDTVENLIILANNS |
| | | | LSSNGNVTESGCKECEELEEKNIKEFLQSFVH |
| | | | IVQMFINTSGGGSIPVSLRSGGGGSSGGSGGG |
| | | | AVNGTSQFTCFYNSRANISCVWSQDGALQDT |
| | | | SCQVHAWPDRRRWNQTCELLPVSQASWACN |
| | | | LILGAPDSQKLTTVDIVTLRVLCREGVRWRV |
| | | | MAIQDFKPFENLRLMAPISLQVVHVETHRCN |
| | | | ISWEISQASHYFERHLEFEARTLSPGHTWEEA |
| | | | PLLTLKQKQEWICLETLTPDTQYEFQVRVKPL |
| | | | QGEFTTWSPWSQGGGGSEPKSSDKTHTCPPC |
| | | | PAPELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEYK |
| | | | CKVSNKALPAPIEKTISKAKGQPREPQVYTLP |
| | | | PSRDELTKNQVSLTCLVKGFYPSDIAVEWESN |
| | | | GQPENNYKTTPPVLDSDGSFFLYSKLTVDKS |
| | | | RWQQGNVFSCSVMHEALHNHYTQKSLSLSP |
| | | | GK |
| | SEQ ID | M244 | ITCPPPMSVEHADIWVKSYSLYSRERYICNSG |
| | NO: 21 | | FKRKAGTSSLTECVLNKATNVAHWTTPSLKC |
| | | | IRDPSGGSGGGGSGGGSGGGGSLQNWVNVI |
| | | | SDLKKIEDLIQSMHIDATLYTESDVHPSCKVT |
| | | | AMKCFLLELQVISLESGDASIHDTVENLIILA |
| | | | NNSLSSNGNVTESGCKECEELEEKNIKEFLQS |
| | | | FVHIVQMFINTSGGGSIPVSLRSGGGGSSGGS |
| | | | GGSGGGTSQFTCFYNSRANISCVWSQDGAL |
| | | | QDTSCQVHAWPDRRRWNQTCELLPVSQAS |
| | | | WACNLILGAPDSQKLTTVDIVTLRVLCREGV |
| | | | RWRVMAIQDFKPFENLRLMAPISLQVVHVET |
| | | | HRCNISWEISQASHYFERHLEFEARTLSPGHT |
| | | | WEEAPLLTLKQKQEWITLETLTPDTQYEFQV |
| | | | RVKPLQGEFTTWSPWSQPLAFRTKPAALGGG |
| | | | GSEPKSSDKTHTCPPCPAPELLGGPSVFLFPP |
| | | | KPKDTLMISRTPEVTCVVVDVSHEDPEVKFN |
| | | | WYVDGVEVHNAKTKPREEQYNSTYRVVSV |
| | | | LTVLHQDWLNGKEYKCKVSNKALPAPIEKTI |
| | | | SKAKGQPREPQVYTLPPSRDELTKNQVSLTC |
| | | | LVKGFYPSDIAVEWESNGQPENNYKTTPPVL |
| | | | DSDGSFFLYSKLTVDKSRWQQGNVFSCSVM |
| | | | HEALHNHYTQKSLSLSPG |
| | SEQ ID | M245 | ITCPPPMSVEHADIWVKSYSLYSRERYICNSG |
| | NO: 22 | | FKRKAGTSSLTECVLNKATNVAHWTTPSLKC |
| | | | IRDPALVHQRSGGSGGGGSGGGSGGGGSLQN |
| | | | WVNVISDLKKIEDLIQSMHIDATLYTESDVHP |
| | | | SCKVTAMKCFLLELQVISLESGDASIHDTVEN |
| | | | LIILANNSLSSNGNVTESGCKECEELEEKNIK |
| | | | EFLQSFVHIVQMFINTSGGGSIPVSLRSGGGG |
| | | | SSGGSGGSGGGTSQFTCFYNSRANISCVWSQ |
| | | | DGALQDTSCQVHAWPDRRRWNQTCELLPVS |
| | | | QASWACNLILGAPDSQKLTTVDIVTLRVLCR |
| | | | EGVRWRVMAIQDFKPFENLRLMAPISLQVVH |
| | | | VETHRCNISWEISQASHYFERHLEFEARTLSP |
| | | | GHTWEEAPLLTLKQKQEWITLETLTPDTQYE |
| | | | FQVRVKPLQGEFTTWSPWSQPLAFRTKPAGG |
| | | | GGSEPKSSDKTHTCPPCPAPELLGGPSVFLFP |
| | | | PKPKDTLMISRTPEVTCVVVDVSHEDPEVKF |
| | | | NWYVDGVEVHNAKTKPREEQYNSTYRVVS |
| | | | VLTVLHQDWLNGKEYKCKVSNKALPAPIEK |
| | | | TISKAKGQPREPQVYTLPPSRDELTKNQVSLT |
| | | | CLVKGFYPSDIAVEWESNGQPENNYKTTPPV |
| | | | LDSDGSFFLYSKLTVDKSRWQQGNVFSCSVM |
| | | | HEALHNHYTQKSLSLSPG |
| | SEQ ID | M246 | ITCPPPMSVEHADIWVKSYSLYSRERYICNSG |
| | NO: 23 | | FKRKAGTSSLTECVLNKATNVAHWTTPSLKC |
| | | | IRDPALVHQRPAPPSGGSGGGGSGGGSGGGG |
| | | | SLQNWVNVISDLKKIEDLIQSMHIDATLYTES |
| | | | DVHPSCKVTAMKCFLLELQVISLESGDASIH |
| | | | DTVENLIILANNSLSSNGNVTESGCKECEELE |
| | | | EKNIKEFLQSFVHIVQMFINTSGGGSIPVSLRS |
| | | | GGGGSSGGSGGSGGGTSQFTCFYNSRANISC |
| | | | VWSQDGALQDTSCQVHAWPDRRRWNQTCE |
| | | | LLPVSQASWACNLILGAPDSQKLTTVDIVTLR |
| | | | VLCREGVRWRVMAIQDFKPFENLRLMAPISL |
| | | | QVVHVETHRCNISWEISQASHYFERHLEFEA |
| | | | RTLSPGHTWEEAPLLTLKQKQEWITLETLTPD |
| | | | TQYEFQVRVKPLQGGGGSEPKSSDKTHTCPP |
| | | | CPAPELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEYK |
| | | | CKVSNKALPAPIEKTISKAKGQPREPQVYTLP |
| | | | PSRDELTKNQVSLTCLVKGFYPSDIAVEWESN |
| | | | GQPENNYKTTPPVLDSDGSFFLYSKLTVDKS |
| | | | RWQQGNVFSCSVMHEALHNHYTQKSLSLSP |
| | | | G |
| | SEQ ID | M247 | ITCPPPMSVEHADIWVKSYSLYSRERYICNSG |
| | NO: 24 | | FKRKAGTSSLTECVLNKATNVAHWTTPSLKC |
| | | | IRDPALVHQRPAPPSGGSGGGGSGGGSGGGG |
| | | | SLQNWVNVISDLKKIEDLIQSMHIDATLYTES |
| | | | DVHPSCKVTAMKCFLLELQVISLESGDASIH |
| | | | DTVENLIILANNSLSSNGNVTESGCKECEELE |
| | | | EKNIKEFLQSFVHIVQMFINTSGGGSIPVSLRS |
| | | | GGGGSSGGSGGSGGGTSQFTCFYNSRANISC |
| | | | VWSQDGALQDTSCQVHAWPDRRRWNQTCE |
| | | | LLPVSQASWACNLILGAPDSQKLTTVDIVTLR |
| | | | VLCREGVRWRVMAIQDFKPFENLRLMAPISL |
| | | | QVVHVETHRCNISWEISQASHYFERHLEFEA |
| | | | RTLSPGHTWEEAPLLTLKQKQEWITLETLTPD |
| | | | TQYEFQVRVKPLQGEFTTWSPWSQPLAFRTK |
| | | | PAGGGGSEPKSSDKTHTCPPCPAPELLGGPSV |
| | | | FLFPPKPKDTLMISRTPEVTCVVVDVSHEDPE |
| | | | VKFNWYVDGVEVHNAKTKPREEQYNSTYR |
| | | | VVSVLTVLHQDWLNGKEYKCKVSNKALPAP |
| | | | IEKTISKAKGQPREPQVYTLPPSRDELTKNQV |
| | | | SLTCLVKGFYPSDIAVEWESNGQPENNYKTT |
| | | | PPVLDSDGSFFLYSKLTVDKSRWQQGNVFSC |
| | | | SVMHEALHNHYTQKSLSLSPG |
| | SEQ ID | M248 | ITCPPPMSVEHADIWVKSYSLYSRERYICNSG |
| | NO: 25 | | FKRKAGTSSLTECVLNKATNVAHWTTPSLKC |
| | | | IRDPALVHQRPAPPSGGSGGGGSGGGSGGGG |
| | | | SLQNWVNVISDLKKIEDLIQSMHIDATLYTES |
| | | | DVHPSCKVTAMKCFLLELQVISLESGDASIH |
| | | | DTVENLIILANNSLSSNGNVTESGCKECEELE |
| | | | EKNIKEFLQSFVHIVQMFINTSGGGSIPVSLRS |
| | | | GGGGSSGGSGGSGGGTSQFTCFYNSRANISC |
| | | | VWSQDGALQDTSCQVHAWPDRRRWNQTCE |
| | | | LLPVSQASWACNLILGAPDSQKLTTVDIVTLR |
| | | | VLCREGVRWRVMAIQDFKPFENLRLMAPISL |
| | | | QVVHVETHRCNISWEISQASHYFERHLEFEA |
| | | | RTLSPGHTWEEAPLLTLKQKQEWITLETLTPD |
| | | | TQYEFQVRVKPLQGEFTTWSPWSQPLAFRTK |
| | | | PAGGGGSGGGGSDKTHTCPPCPAPELLGGPS |
| | | | VFLFPPKPKDTLMISRTPEVTCVVVDVSHEDP |
| | | | EVKFNWYVDGVEVHNAKTKPREEQYNSTY |
| | | | RVVSVLTVLHQDWLNGKEYKCKVSNKALPA |
| | | | PIEKTISKAKGQPREPQVYTLPPSRDELTKNQ |
| | | | VSLTCLVKGFYPSDIAVEWESNGQPENNYKT |
| | | | TPPVLDSDGSFFLYSKLTVDKSRWQQGNVFS |
| | | | CSVMHEALHNHYTQKSLSLSPG |
| | SEQ ID | M249 | ITCPPPMSVEHADIWVKSYSLYSRERYICNSG |
| | NO: 26 | | FKRKAGTSSLTECVLNKATNVAHWTTPSLKC |
| | | | IRDPALVHQRPAPPSGGSGGGGSGGGSGGGG |
| | | | SLQNWVNVISDLKKIEDLIQSMHIDATLYTES |
| | | | DVHPSCKVTAMKCFLLELQVISLESGDASIH |
| | | | DTVENLIILANNSLSSNGNVTESGCKECEELE |
| | | | EKNIKEFLQSFVHIVQMFINTSGGGSIPVSLRS |
| | | | GGGGSSGGSGGSGGGTSQFTCFYNSRANISC |
| | | | VWSQDGALQDTSCQVHAWPDRRRWNQTCE |
| | | | LLPVSQASWACNLILGAPDSQKLTTVDIVTLR |
| | | | VLCREGVRWRVMAIQDFKPFENLRLMAPISL |
| | | | QVVHVETHRCNISWEISQASHYFERHLEFEA |
| | | | RTLSPGHTWEEAPLLTLKQKQEWITLETLTPD |
| | | | TQYEFQVRVKPLQGEFTTWSPWSQPLAFRTK |
| | | | PAGGGGSGGGGSGGGGSDKTHTCPPCPAPEL |
| | | | LGGPSVFLFPPKPKDTLMISRTPEVTCVVVDV |
| | | | SHEDPEVKFNWYVDGVEVHNAKTKPREEQ |
| | | | YNSTYRVVSVLTVLHQDWLNGKEYKCKVSN |
| | | | KALPAPIEKTISKAKGQPREPQVYTLPPSRDE |
| | | | LTKNQVSLTCLVKGFYPSDIAVEWESNGQPE |
| | | | NNYKTTPPVLDSDGSFFLYSKLTVDKSRWQQ |
| | | | GNVFSCSVMHEALHNHYTQKSLSLSPG |
| | SEQ ID | M327 | ITCPPPMSVEHADIWVKSYSLYSRERYICNSG |
| | NO: 27 | | FKRKAGTSSLTECVLNKATNVAHWTTPSLKC |
| | | | IRDPALVHQRPAPPSGGSGGGGSGGGSGGGG |
| | | | SLQNWVNVISDLKKIEDLIQSMHIDATLYTES |
| | | | DVHPSCKVTAMKCFLLELQVISLESGDASIH |
| | | | DTVENLIILANNSLSSNGNVTESGCKECEELE |
| | | | EKNIKEFLQSFVHIVQMFINTSGGGSIPVSLRS |
| | | | GGGGSSGGSGGSGGAVNGTSQFTCFYNSRA |
| | | | NISCVWSQDGALQDTSCQVHAWPDRRRWN |
| | | | QTCELLPVSQASWACNLILGAPDSQKLTTVDI |
| | | | VTLRVLCREGVRWRVMAIQDFKPFENLRLM |
| | | | APISLQVVHVETHRCNISWEISQASHYFERHL |
| | | | EFEARTLSPGHTWEEAPLLTLKQKQEWICLE |
| | | | TLTPDTQYEFQVRVKPLQGEFTTWSPWSQPL |
| | | | AFRTKPAALGKDTGGGGSEPKSSDKTHTCPP |
| | | | CPAPELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEYK |
| | | | CKVSNKALPAPIEKTISKAKGQPREPQVYTLP |
| | | | PSRDELTKNQVSLTCLVKGFYPSDIAVEWESN |
| | | | GQPENNYKTTPPVLDSDGSFFLYSKLTVDKS |
| | | | RWQQGNVFSCSVMHEALHNHYTQKSLSLSP |
| | | | GK |
| | SEQ ID | M328 | ITCPPPMSVEHADIWVKSYSLYSRERYICNSG |
| | NO: 28 | | FKRKAGTSSLTECVLNKATNVAHWTTPSLKC |
| | | | IRDPALVHQRPAPPSGGSGGGGSGGGSGGGG |
| | | | SLQNWVNVISDLKKIEDLIQSMHIDATLYTES |
| | | | DVHPSCKVTAMKCFLLELQVISLESGDASIH |
| | | | DTVENLIILANNSLSSNGNVTESGCKECEELE |
| | | | EKNIKEFLQSFVHIVQMFINTSGGGSIPVSLRS |
| | | | GGGGSSGGSGGSGGAVNGTSQFTCFYNSRA |
| | | | NISCVWSQDGALQDTSCQVHAWPDRRRWN |
| | | | QTCELLPVSQASWACNLILGAPDSQKLTTVDI |
| | | | VTLRVLCREGVRWRVMAIQDFKPFENLRLM |
| | | | APISLQVVHVETHRCNISWEISQASHYFERHL |
| | | | EFEARTLSPGHTWEEAPLLTLKQKQEWICLE |
| | | | TLTPDTQYEFQVRVKPLQGEFTTWSPWSQPL |
| | | | AFRTKPAALGKDTAPAPAPEPKSSDKTHTCPP |
| | | | CPAPELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEYK |
| | | | CKVSNKALPAPIEKTISKAKGQPREPQVYTLP |
| | | | PSRDELTKNQVSLTCLVKGFYPSDIAVEWESN |
| | | | GQPENNYKTTPPVLDSDGSFFLYSKLTVDKS |
| | | | RWQQGNVFSCSVMHEALHNHYTQKSLSLSP |
| | | | GK |
| | SEQ ID | M329 | ITCPPPMSVEHADIWVKSYSLYSRERYICNSG |
| | NO: 29 | | FKRKAGTSSLTECVLNKATNVAHWTTPSLKC |
| | | | IRDPALVHQRPAPPSGGSGGGGSGGGSGGGG |
| | | | SLQNWVNVISDLKKIEDLIQSMHIDATLYTES |
| | | | DVHPSCKVTAMKCFLLELQVISLESGDASIH |
| | | | DTVENLIILANNSLSSNGNVTESGCKECEELE |
| | | | EKNIKEFLQSFVHIVQMFINTSGGGSIPVSLRS |
| | | | GGGGSSGGSGGSGGAVNGTSQFTCFYNSRA |
| | | | NISCVWSQDGALQDTSCQVHAWPDRRRWN |
| | | | QTCELLPVSQASWACNLILGAPDSQKLTTVDI |
| | | | VTLRVLCREGVRWRVMAIQDFKPFENLRLM |
| | | | APISLQVVHVETHRANISWEISQASHYFERHL |
| | | | EFEARTLSPGHTWEEAPLLTLKQKQEWITLE |
| | | | TLTPDTQYEFQVRVKPLQGEFTTWSPWSQPL |
| | | | AFRTKPAALGKDTAPAPAPEPKSSDKTHTCPP |
| | | | CPAPELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEYK |
| | | | CKVSNKALPAPIEKTISKAKGQPREPQVYTLP |
| | | | PSRDELTKNQVSLTCLVKGFYPSDIAVEWESN |
| | | | GQPENNYKTTPPVLDSDGSFFLYSKLTVDKS |
| | | | RWQQGNVFSCSVMHEALHNHYTQKSLSLSP |
| | | | GK |
| | SEQ ID | M330 | ITCPPPMSVEHADIWVKSYSLYSRERYICNSG |
| | NO: 30 | | FKRKAGTSSLTECVLNKATNVAHWTTPSLKC |
| | | | IRDPALVHQRPAPPSGGSGGGGSGGGSGGGG |
| | | | SLQNWVNVISDLKKIEDLIQSMHIDATLYTES |
| | | | DVHPSCKVTAMKCFLLELQVISLESGDASIH |
| | | | DTVENLIILANNSLSSNGNVTESGCKECEELE |
| | | | EKNIKEFLQSFVHIVQMFINTSGGGSVPLSLY |
| | | | SGRSASGGSGGGGSGSGAVNGTSQFTCFYNS |
| | | | RANISCVWSQDGALQDTSCQVHAWPDRRR |
| | | | WNQTCELLPVSQASWACNLILGAPDSQKLTT |
| | | | VDIVTLRVLCREGVRWRVMAIQDFKPFENLR |
| | | | LMAPISLQVVHVETHRCNISWEISQASHYFE |
| | | | RHLEFEARTLSPGHTWEEAPLLTLKQKQEWI |
| | | | CLETLTPDTQYEFQVRVKPLQGEFTTWSPWS |
| | | | QPLAFRTKPAALGKDTGGGGSEPKSSDKTHT |
| | | | CPPCPAPELLGGPSVFLFPPKPKDTLMISRTPE |
| | | | VTCVVVDVSHEDPEVKFNWYVDGVEVHNA |
| | | | KTKPREEQYNSTYRVVSVLTVLHQDWLNGK |
| | | | EYKCKVSNKALPAPIEKTISKAKGQPREPQV |
| | | | YTLPPSRDELTKNQVSLTCLVKGFYPSDIAVE |
| | | | WESNGQPENNYKTTPPVLDSDGSFFLYSKLT |
| | | | VDKSRWQQGNVFSCSVMHEALHNHYTQKS |
| | | | LSLSPGK |
| | SEQ ID | M331 | ITCPPPMSVEHADIWVKSYSLYSRERYICNSG |
| | NO: 31 | | FKRKAGTSSLTECVLNKATNVAHWTTPSLKC |
| | | | IRDPALVHQRPAPPSGGSGGGGSGGGSGGGG |
| | | | SLQNWVNVISDLKKIEDLIQSMHIDATLYTES |
| | | | DVHPSCKVTAMKCFLLELQVISLESGDASIH |
| | | | DTVENLIILANNSLSSNGNVTESGCKECEELE |
| | | | EKNIKEFLQSFVHIVQMFINTSGGGSVPLSLY |
| | | | SGRSASGGSGGGGSGSGAVNGTSQFTCFYNS |
| | | | RANISCVWSQDGALQDTSCQVHAWPDRRR |
| | | | WNQTCELLPVSQASWACNLILGAPDSQKLTT |
| | | | VDIVTLRVLCREGVRWRVMAIQDFKPFENLR |
| | | | LMAPISLQVVHVETHRCNISWEISQASHYFE |
| | | | RHLEFEARTLSPGHTWEEAPLLTLKQKQEWI |
| | | | CLETLTPDTQYEFQVRVKPLQGEFTTWSPWS |
| | | | QPLAFRTKPAALGKDTEAAAKEPKSSDKTHT |
| | | | CPPCPAPELLGGPSVFLFPPKPKDTLMISRTPE |
| | | | VTCVVVDVSHEDPEVKFNWYVDGVEVHNA |
| | | | KTKPREEQYNSTYRVVSVLTVLHQDWLNGK |
| | | | EYKCKVSNKALPAPIEKTISKAKGQPREPQV |
| | | | YTLPPSRDELTKNQVSLTCLVKGFYPSDIAVE |
| | | | WESNGQPENNYKTTPPVLDSDGSFFLYSKLT |
| | | | VDKSRWQQGNVFSCSVMHEALHNHYTQKS |
| | | | LSLSPGK |
| | SEQ ID | M332 | ITCPPPMSVEHADIWVKSYSLYSRERYICNSG |
| | NO: 32 | | FKRKAGTSSLTECVLNKATNVAHWTTPSLKC |
| | | | IRDPALVHQRPAPPSGGSGGGGSGGGSGGGG |
| | | | SLQNWVNVISDLKKIEDLIQSMHIDATLYTES |
| | | | DVHPSCKVTAMKCFLLELQVISLESGDASIH |
| | | | DTVENLIILANNSLSSNGNVTESGCKECEELE |
| | | | EKNIKEFLQSFVHIVQMFINTSGGGSVPLSLY |
| | | | SGRSASGGSGGGGSGSGAVNGTSQFTCFYNS |
| | | | RANISCVWSQDGALQDTSCQVHAWPDRRR |
| | | | WNQTCELLPVSQASWACNLILGAPDSQKLTT |
| | | | VDIVTLRVLCREGVRWRVMAIQDFKPFENLR |
| | | | LMAPISLQVVHVETHRANISWEISQASHYFE |
| | | | RHLEFEARTLSPGHTWEEAPLLTLKQKQEWI |
| | | | TLETLTPDTQYEFQVRVKPLQGEFTTWSPWS |
| | | | QPLAFRTKPAALGKDTEAAAKEPKSSDKTHT |
| | | | CPPCPAPELLGGPSVFLFPPKPKDTLMISRTPE |
| | | | VTCVVVDVSHEDPEVKFNWYVDGVEVHNA |
| | | | KTKPREEQYNSTYRVVSVLTVLHQDWLNGK |
| | | | EYKCKVSNKALPAPIEKTISKAKGQPREPQV |
| | | | YTLPPSRDELTKNQVSLTCLVKGFYPSDIAVE |
| | | | WESNGQPENNYKTTPPVLDSDGSFFLYSKLT |
| | | | VDKSRWQQGNVFSCSVMHEALHNHYTQKS |
| | | | LSLSPGK |
| Construct B | SEQ ID | M43 | AVNGTSQFTCFYNSRANISCVWSQDGALQDT |
| | NO: 33 | pairs | SCQVHAWPDRRRWNQTCELLPVSQASWACN |
| | | with | LILGAPDSQKLTTVDIVTLRVLCREGVRWRV |
| | | M24 | MAIQDFKPFENLRLMAPISLQVVHVETHRCN |
| | | (SEQ ID | ISWEISQASHYFERHLEFEARTLSPGHTWEEA |
| | | NO: 34) | PLLTLKQKQEWICLETLTPDTQYEFQVRVKPL |
| | | | QGEFTTWSPWSQPLAFRTKPAALGKDGGGSS |
| | | | GGSGGSGGGSGGGSLSGRSDNHGGSGNWV |
| | | | NVISDLKKIEDLIQSMHIDATLYSESDVHPSC |
| | | | KVTAMKCFLLEFQVISCESGDASIHDTVENLI |
| | | | ILANDSLSSNGNVTESGCKECEELEEKNIKEF |
| | | | LQSFVHIVQMFINTS |
| | SEQ ID | M24 | ITCPPPMSVEHADIWVKSYSLYSRERYICNSG |
| | NO: 34 | | FKRKAGTCSLTECVLNKATNVAHWTTPSLKC |
| | | | IRDPALVHQREPKSCDKTHTCPPCPAPELLGG |
| | | | PSVFLFPPKPKDTLMISRTPEVTCVVVDVSHE |
| | | | DPEVKFNWYVDGVEVHNAKTKPREEQYNS |
| | | | TYRVVSVLTVLHQDWLNGKEYKCKVSNKA |
| | | | LPAPIEKTISKAKGQPREPQVYTLPPSRDELTK |
| | | | NQVSLTCLVKGFYPSDIAVEWESNGQPENNY |
| | | | KTTPPVLDSDGSFFLYSKLTVDKSRWQQGNV |
| | | | FSCSVMHEALHNHYTQKSLSLSPGK |
| | SEQ ID | M61 | GTSQFTCFYNSRANISCVWSQDGALQDTSCQ |
| | NO: 35 | pairs | VHAWPDRRRWNQTCELLPVSQASWACNLIL |
| | | with | GAPDSQKLTTVDIVTLRVLCREGVRWRVMAI |
| | | M60 | QDFKPFENLRLMAPISLQVVHVETHRANISW |
| | | (SEQ ID | EISQASHYFERHLEFEARTLSPGHTWEEAPLL |
| | | NO: 36) | TLKQKQEWISLETLTPDTQYEFQVRVKPLQG |
| | | | EFTTWSPWSQPLAFRTKPAALGKDGGGSIPV |
| | | | SLRSGGGGSSGGSGGSGGNWVNVISDLKKIE |
| | | | DLIQSMHIDATLYSESDVHPSCKVTAMKCFLL |
| | | | EFQVISCESGDASIHDTVENLIILANDSLSSNG |
| | | | NVTESGCKECEELEEKNIKEFLQSFVHIVQMF |
| | | | INTS |
| | SEQ ID | M60 | ITCPPPMSVEHADIWVKSYSLYSRERYICNSG |
| | NO: 36 | | FKRKAGTCSLTECVLNKATNVAHWTTPSLKC |
| | | | IRDPALVHQREPKSSDKTHTCPPCPAPELLGG |
| | | | PSVFLFPPKPKDTLMISRTPEVTCVVVDVSHE |
| | | | DPEVKFNWYVDGVEVHNAKTKPREEQYNS |
| | | | TYRVVSVLTVLHQDWLNGKEYKCKVSNKA |
| | | | LPAPIEKTISKAKGQPREPQVYTLPPSRDELTK |
| | | | NQVSLTCLVKGFYPSDIAVEWESNGQPENNY |
| | | | KTTPPVLDSDGSFFLYSKLTVDKSRWQQGNV |
| | | | FSCSVMHEALHNHYTQKSLSLSPGK |
| | SEQ ID | M62 | AVNGTSQFTCFYNSRANISCVWSQDGALQDT |
| | NO: 37 | pairs | SCQVHAWPDRRRWNQTCELLPVSQASWACN |
| | | with | LILGAPDSQKLTTVDIVTLRVLCREGVRWRV |
| | | M60 | MAIQDFKPFENLRLMAPISLQVVHVETHRAN |
| | | (SEQ ID | ISWEISQASHYFERHLEFEARTLSPGHTWEEA |
| | | NO: 38) | PLLTLKQKQEWISLETLTPDTQYEFQVRVKPL |
| | | | QGEFTTWSPWSQPLAFRTKPAALGKDGGGSI |
| | | | SSGLLSGRSDNHGGGSSGGSNWVNVISDLK |
| | | | KIEDLIQSMHIDATLYTESDVHPSCKVTAMKC |
| | | | FLLELQVISCESGDASIHDTVENLIILANDSLS |
| | | | SNGNVTESGCKECEELEEKNIKEFLQSFVHIV |
| | | | QMFINTS |
| | SEQ ID | M60 | ITCPPPMSVEHADIWVKSYSLYSRERYICNSG |
| | NO: 38 | | FKRKAGTCSLTECVLNKATNVAHWTTPSLKC |
| | | | IRDPALVHQREPKSSDKTHTCPPCPAPELLGG |
| | | | PSVFLFPPKPKDTLMISRTPEVTCVVVDVSHE |
| | | | DPEVKFNWYVDGVEVHNAKTKPREEQYNS |
| | | | TYRVVSVLTVLHQDWLNGKEYKCKVSNKA |
| | | | LPAPIEKTISKAKGQPREPQVYTLPPSRDELTK |
| | | | NQVSLTCLVKGFYPSDIAVEWESNGQPENNY |
| | | | KTTPPVLDSDGSFFLYSKLTVDKSRWQQGNV |
| | | | FSCSVMHEALHNHYTQKSLSLSPGK |
| Construct C | SEQ ID | M101 | AVNGTSQFTCFYNSRANISCVWSQDGALQDT |
| | NO: 39 | | SCQVHAWPDRRRWNQTCELLPVSQASWACN |
| | | | LILGAPDSQKLTTVDIVTLRVLCREGVRWRV |
| | | | MAIQDFKPFENLRLMAPISLQVVHVETHRCN |
| | | | ISWEISQASHYFERHLEFEARTLSPGHTWEEA |
| | | | PLLTLKQKQEWICLETLTPDTQYEFQVRVKPL |
| | | | QGEFTTWSPWSQPLAFRTKPAALGKDGGGSS |
| | | | GGSGGSGGSGGGSGGGSLSGRSDNHGGSGN |
| | | | WVNVISDLKKIEDLIQSMHIDATLYTESDVHP |
| | | | SCKVTAMKCFLLELQVISLESGDASIHDTVEN |
| | | | LIILANNSLSSNGNVTESGCKECEELEEKNIK |
| | | | EFLQSFVHIVQMFINTSSGGSGGGGSGGGSG |
| | | | GGGSLQITCPPPMSVEHADIWVKSYSLYSRE |
| | | | RYICNSGFKRKAGTSSLTECVLNKATNVAHW |
| | | | TTPSLKCIREPKSCDKTHTCPPCPAPELLGGPS |
| | | | VFLFPPKPKDTLMISRTPEVTCVVVDVSHEDP |
| | | | EVKFNWYVDGVEVHNAKTKPREEQYNSTY |
| | | | RVVSVLTVLHQDWLNGKEYKCKVSNKALPA |
| | | | PIEKTISKAKGQPREPQVYTLPPSRDELTKNQ |
| | | | VSLTCLVKGFYPSDIAVEWESNGQPENNYKT |
| | | | TPPVLDSDGSFFLYSKLTVDKSRWQQGNVFS |
| | | | CSVMHEALHNHYTQKSLSLSPGK |
| | SEQ ID | M148 | AVNGTSQFTCFYNSRANISCVWSQDGALQDT |
| | NO: 40 | | SCQVHAWPDRRRWNQTCELLPVSQASWACN |
| | | | LILGAPDSQKLTTVDIVTLRVLCREGVRWRV |
| | | | MAIQDFKPFENLRLMAPISLQVVHVETHRCN |
| | | | ISWEISQASHYFERHLEFEARTLSPGHTWEEA |
| | | | PLLTLKQKQEWICLETLTPDTQYEFQVRVKPL |
| | | | QGEFTTWSPWSQPLAFRTKPAALGKDGGGSS |
| | | | GGSGGSGGIPVSLRSGGGGSNWVNVISDLKK |
| | | | IEDLIQSMHIDATLYTESDVHPSCKVTAMKCF |
| | | | LLELQVISLESGDASIHDTVENLIILANNSLSS |
| | | | NGNVTESGCKECEELEEKNIKEFLQSFVHIVQ |
| | | | MFINTSGGGSGGGGSGGGGSGGGGSGGGSL |
| | | | QITCPPPMSVEHADIWVKSYSLYSRERYICNS |
| | | | GFKRKAGTSSLTECVLNKATNVAHWTTPSLK |
| | | | CIREPKSCDKTHTCPPCPAPELLGGPSVFLFPP |
| | | | KPKDTLMISRTPEVTCVVVDVSHEDPEVKFN |
| | | | WYVDGVEVHNAKTKPREEQYNSTYRVVSV |
| | | | LTVLHQDWLNGKEYKCKVSNKALPAPIEKTI |
| | | | SKAKGQPREPQVYTLPPSRDELTKNQVSLTC |
| | | | LVKGFYPSDIAVEWESNGQPENNYKTTPPVL |
| | | | DSDGSFFLYSKLTVDKSRWQQGNVFSCSVM |
| | | | HEALHNHYTQKSLSLSPGK |
| | SEQ ID | M174 | AVNGTSQFTCFYNSRANISCVWSQDGALQDT |
| | NO: 41 | | SCQVHAWPDRRRWNQTCELLPVSQASWACN |
| | | | LILGAPDSQKLTTVDIVTLRVLCREGVRWRV |
| | | | MAIQDFKPFENLRLMAPISLQVVHVETHRCN |
| | | | ISWEISQASHYFERHLEFEARTLSPGHTWEEA |
| | | | PLLTLKQKQEWICLETLTPDTQYEFQVRVKPL |
| | | | QGEFTTWSPWSQPLAFRTKPAALGKDGGGG |
| | | | SSGRIGFLRTAGGGGSNWVNVISDLKKIEDLI |
| | | | QSMHIDATLYTESDVHPSCKVTAMKCFLLEL |
| | | | QVISLESGDASIHDTVENLIILANNSLSSNGN |
| | | | VTESGCKECEELEEKNIKEFLQSFVHIVQMFI |
| | | | NTSSGGGSGGGGSGGGGSGGGGSGGGSLQI |
| | | | TCPPPMSVEHADIWVKSYSLYSRERYICNSGF |
| | | | KRKAGTSSLTECVLNKATNVAHWTTPSLKCI |
| | | | RDPALVHQRPAPPGGGGSEPKSSDKTHTCPP |
| | | | CPAPELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEYK |
| | | | CKVSNKALPAPIEKTISKAKGQPREPQVYTLP |
| | | | PSRDELTKNQVSLTCLVKGFYPSDIAVEWESN |
| | | | GQPENNYKTTPPVLDSDGSFFLYSKLTVDKS |
| | | | RWQQGNVFSCSVMHEALHNHYTQKSLSLSP |
| | | | GK |
| Construct D | SEQ ID | M232 | DKTHTCPPCPAPELLGGPSVFLFPPKPKDTLM |
| | NO: 42 | | ISRTPEVTCVVVDVSHEDPEVKFNWYVDGV |
| | | | EVHNAKTKPREEQYNSTYRVVSVLTVLHQD |
| | | | WLNGKEYKCKVSNKALPAPIEKTISKAKGQP |
| | | | REPQVYTLPPSRDELTKNQVSLTCLVKGFYPS |
| | | | DIAVEWESNGQPENNYKTTPPVLDSDGSFFL |
| | | | YSKLTVDKSRWQQGNVFSCSVMHEALHNH |
| | | | YTQKSLSLSPGKGGGGSAVNGTSQFTCFYNS |
| | | | RANISCVWSQDGALQDTSCQVHAWPDRRR |
| | | | WNQTCELLPVSQASWACNLILGAPDSQKLTT |
| | | | VDIVTLRVLCREGVRWRVMAIQDFKPFENLR |
| | | | LMAPISLQVVHVETHRCNISWEISQASHYFE |
| | | | RHLEFEARTLSPGHTWEEAPLLTLKQKQEWI |
| | | | CLETLTPDTQYEFQVRVKPLQGEFTTWSPWS |
| | | | QPLAFRTKPAALGKDGGGSIPVSLRSGGGGS |
| | | | SGGSGGSGGITCPPPMSVEHADIWVKSYSLY |
| | | | SRERYICNSGFKRKAGTSSLTECVLNKATNVA |
| | | | HWTTPSLKCIRSGGSGGGGSGGGSGGGGSL |
| | | | QNWVNVISDLKKIEDLIQSMHIDATLYTESDV |
| | | | HPSCKVTAMKCFLLELQVISLESGDASIHDTV |
| | | | ENLIILANNSLSSNGNVTESGCKECEELEEKN |
| | | | IKEFLQSFVHIVQMFINTS |
| | SEQ ID | M1001 | DKTHTCPPCPAPELLGGPSVFLFPPKPKDTLM |
| | NO: 43 | | ISRTPEVTCVVVDVSHEDPEVKFNWYVDGV |
| | | | EVHNAKTKPREEQYNSTYRVVSVLTVLHQD |
| | | | WLNGKEYKCKVSNKALPAPIEKTISKAKGQP |
| | | | REPQVYTLPPSRDELTKNQVSLTCLVKGFYPS |
| | | | DIAVEWESNGQPENNYKTTPPVLDSDGSFFL |
| | | | YSKLTVDKSRWQQGNVFSCSVMHEALHNH |
| | | | YTQKSLSLSPGGGGGSAVNGTSQFTCFYNSR |
| | | | ANISCVWSQDGALQDTSCQVHAWPDRRRW |
| | | | NQTCELLPVSQASWACNLILGAPDSQKLTTV |
| | | | DIVTLRVLCREGVRWRVMAIQDFKPFENLRL |
| | | | MAPISLQVVHVETHRCNISWEISQASHYFER |
| | | | HLEFEARTLSPGHTWEEAPLLTLKQKQEWIC |
| | | | LETLTPDTQYEFQVRVKPLQGEFTTWSPWSQ |
| | | | PLAFRTKPAALGKDGGGSSGGSGGSGGIPVS |
| | | | LRSGGGGSITCPPPMSVEHADIWVKSYSLYS |
| | | | RERYICNSGFKRKAGTSSLTECVLNKATNVA |
| | | | HWTTPSLKCIRDPALVHQRPAPPSGGSGGGG |
| | | | SGGGSGGGGSLQNWVNVISDLKKIEDLIQSM |
| | | | HIDATLYTESDVHPSCKVTAMKCFLLELQVIS |
| | | | LESGDASIHDTVENLIILANNSLSSNGNVTES |
| | | | GCKECEELEEKNIKEFLQSFVHIVQMFINTS |
| | SEQ ID | M1002 | DKTHTCPPCPAPELLGGPSVFLFPPKPKDTLM |
| | NO: 44 | | ISRTPEVTCVVVDVSHEDPEVKFNWYVDGV |
| | | | EVHNAKTKPREEQYNSTYRVVSVLTVLHQD |
| | | | WLNGKEYKCKVSNKALPAPIEKTISKAKGQP |
| | | | REPQVYTLPPSRDELTKNQVSLTCLVKGFYPS |
| | | | DIAVEWESNGQPENNYKTTPPVLDSDGSFFL |
| | | | YSKLTVDKSRWQQGNVFSCSVMHEALHNH |
| | | | YTQKSLSLSPGGGGGSGGGGSAVNGTSQFTC |
| | | | FYNSRANISCVWSQDGALQDTSCQVHAWPD |
| | | | RRRWNQTCELLPVSQASWACNLILGAPDSQ |
| | | | KLTTVDIVTLRVLCREGVRWRVMAIQDFKPF |
| | | | ENLRLMAPISLQVVHVETHRCNISWEISQAS |
| | | | HYFERHLEFEARTLSPGHTWEEAPLLTLKQK |
| | | | QEWICLETLTPDTQYEFQVRVKPLQGEFTTW |
| | | | SPWSQPLAFRTKPAALGKDGGGSSGGSGGSG |
| | | | GIPVSLRSGGGGSITCPPPMSVEHADIWVKSY |
| | | | SLYSRERYICNSGFKRKAGTSSLTECVLNKAT |
| | | | NVAHWTTPSLKCIRDPALVHQRPAPPSGGSG |
| | | | GGGSGGGSGGGGSLQNWVNVISDLKKIEDLI |
| | | | QSMHIDATLYTESDVHPSCKVTAMKCFLLEL |
| | | | QVISLESGDASIHDTVENLIILANNSLSSNGN |
| | | | VTESGCKECEELEEKNIKEFLQSFVHIVQMFI |
| | | | NTS |
| | SEQ ID | M1003 | DKTHTCPPCPAPELLGGPSVFLFPPKPKDTLM |
| | NO: 45 | | ISRTPEVTCVVVDVSHEDPEVKFNWYVDGV |
| | | | EVHNAKTKPREEQYNSTYRVVSVLTVLHQD |
| | | | WLNGKEYKCKVSNKALPAPIEKTISKAKGQP |
| | | | REPQVYTLPPSRDELTKNQVSLTCLVKGFYPS |
| | | | DIAVEWESNGQPENNYKTTPPVLDSDGSFFL |
| | | | YSKLTVDKSRWQQGNVFSCSVMHEALHNH |
| | | | YTQKSLSLSPGGGGGSGGGGSGGGGSAVNG |
| | | | TSQFTCFYNSRANISCVWSQDGALQDTSCQV |
| | | | HAWPDRRRWNQTCELLPVSQASWACNLILG |
| | | | APDSQKLTTVDIVTLRVLCREGVRWRVMAIQ |
| | | | DFKPFENLRLMAPISLQVVHVETHRCNISWEI |
| | | | SQASHYFERHLEFEARTLSPGHTWEEAPLLTL |
| | | | KQKQEWICLETLTPDTQYEFQVRVKPLQGEF |
| | | | TTWSPWSQPLAFRTKPAALGKDGGGSSGGS |
| | | | GGSGGIPVSLRSGGGGSITCPPPMSVEHADIW |
| | | | VKSYSLYSRERYICNSGFKRKAGTSSLTECVL |
| | | | NKATNVAHWTTPSLKCIRDPALVHQRPAPPS |
| | | | GGSGGGGSGGGSGGGGSLQNWVNVISDLKK |
| | | | IEDLIQSMHIDATLYTESDVHPSCKVTAMKCF |
| | | | LLELQVISLESGDASIHDTVENLIILANNSLSS |
| | | | NGNVTESGCKECEELEEKNIKEFLQSFVHIVQ |
| | | | MFINTS |
| | SEQ ID | M1004 | DKTHTCPPCPAPELLGGPSVFLFPPKPKDTLM |
| | NO: 46 | | ISRTPEVTCVVVDVSHEDPEVKFNWYVDGV |
| | | | EVHNAKTKPREEQYNSTYRVVSVLTVLHQD |
| | | | WLNGKEYKCKVSNKALPAPIEKTISKAKGQP |
| | | | REPQVYTLPPSRDELTKNQVSLTCLVKGFYPS |
| | | | DIAVEWESNGQPENNYKTTPPVLDSDGSFFL |
| | | | YSKLTVDKSRWQQGNVFSCSVMHEALHNH |
| | | | YTQKSLSLSPGGGGGSAVNGTSQFTCFYNSR |
| | | | ANISCVWSQDGALQDTSCQVHAWPDRRRW |
| | | | NQTCELLPVSQASWACNLILGAPDSQKLTTV |
| | | | DIVTLRVLCREGVRWRVMAIQDFKPFENLRL |
| | | | MAPISLQVVHVETHRCNISWEISQASHYFER |
| | | | HLEFEARTLSPGHTWEEAPLLTLKQKQEWIC |
| | | | LETLTPDTQYEFQVRVKPLQGEFTTWSPWSQ |
| | | | PLAFRTKPAALGKDTGGGSSGGSGGSGGIPV |
| | | | SLRSGGGGSITCPPPMSVEHADIWVKSYSLYS |
| | | | RERYICNSGFKRKAGTSSLTECVLNKATNVA |
| | | | HWTTPSLKCIRDPALVHQRPAPPSGGSGGGG |
| | | | SGGGSGGGGSLQNWVNVISDLKKIEDLIQSM |
| | | | HIDATLYTESDVHPSCKVTAMKCFLLELQVIS |
| | | | LESGDASIHDTVENLIILANNSLSSNGNVTES |
| | | | GCKECEELEEKNIKEFLQSFVHIVQMFINTS |
| | SEQ ID | M1005 | DKTHTCPPCPAPELLGGPSVFLFPPKPKDTLM |
| | NO: 47 | | ISRTPEVTCVVVDVSHEDPEVKFNWYVDGV |
| | | | EVHNAKTKPREEQYNSTYRVVSVLTVLHQD |
| | | | WLNGKEYKCKVSNKALPAPIEKTISKAKGQP |
| | | | REPQVYTLPPSRDELTKNQVSLTCLVKGFYPS |
| | | | DIAVEWESNGQPENNYKTTPPVLDSDGSFFL |
| | | | YSKLTVDKSRWQQGNVFSCSVMHEALHNH |
| | | | YTQKSLSLSPGGGGGSAVNGTSQFTCFYNSR |
| | | | ANISCVWSQDGALQDTSCQVHAWPDRRRW |
| | | | NQTCELLPVSQASWACNLILGAPDSQKLTTV |
| | | | DIVTLRVLCREGVRWRVMAIQDFKPFENLRL |
| | | | MAPISLQVVHVETHRANISWEISQASHYFER |
| | | | HLEFEARTLSPGHTWEEAPLLTLKQKQEWIT |
| | | | LETLTPDTQYEFQVRVKPLQGEFTTWSPWSQ |
| | | | PLAFRTKPAALGKDTGGGSSGGSGGSGGIPV |
| | | | SLRSGGGGSITCPPPMSVEHADIWVKSYSLYS |
| | | | RERYICNSGFKRKAGTSSLTECVLNKATNVA |
| | | | HWTTPSLKCIRDPALVHQRPAPPSGGSGGGG |
| | | | SGGGSGGGGSLQNWVNVISDLKKIEDLIQSM |
| | | | HIDATLYTESDVHPSCKVTAMKCFLLELQVIS |
| | | | LESGDASIHDTVENLIILANNSLSSNGNVTES |
| | | | GCKECEELEEKNIKEFLQSFVHIVQMFINTS |
| | SEQ ID | M1006 | DKTHTCPPCPAPELLGGPSVFLFPPKPKDTLM |
| | NO: 48 | | ISRTPEVTCVVVDVSHEDPEVKFNWYVDGV |
| | | | EVHNAKTKPREEQYNSTYRVVSVLTVLHQD |
| | | | WLNGKEYKCKVSNKALPAPIEKTISKAKGQP |
| | | | REPQVYTLPPSRDELTKNQVSLTCLVKGFYPS |
| | | | DIAVEWESNGQPENNYKTTPPVLDSDGSFFL |
| | | | YSKLTVDKSRWQQGNVFSCSVMHEALHNH |
| | | | YTQKSLSLSPGGGGGSAVQGTSQFTCFYNSR |
| | | | AQISCVWSQDGALQDTSCQVHAWPDRRRW |
| | | | NQTCELLPVSQASWACNLILGAPDSQKLTTV |
| | | | DIVTLRVLCREGVRWRVMAIQDFKPFENLRL |
| | | | MAPISLQVVHVETHRANISWEISQASHYFER |
| | | | HLEFEARTLSPGHTWEEAPLLTLKQKQEWIT |
| | | | LETLTPDTQYEFQVRVKPLQGEFTTWSPWSQ |
| | | | PLAFRTKPAALGKDTGGGSSGGSGGSGGIPV |
| | | | SLRSGGGGSITCPPPMSVEHADIWVKSYSLYS |
| | | | RERYICNSGFKRKAGTSSLTECVLNKATNVA |
| | | | HWTTPSLKCIRDPALVHQRPAPPSGGSGGGG |
| | | | SGGGSGGGGSLQNWVNVISDLKKIEDLIQSM |
| | | | HIDATLYTESDVHPSCKVTAMKCFLLELQVIS |
| | | | LESGDASIHDTVENLIILAQNSLSSNGNVTES |
| | | | GCKECEELEEKNIKEFLQSFVHIVQMFIQTS |
| Construct | SEQ ID | MK107 | ITCPPPMSVEHADIWVKSYSLYSRERYICNSG |
| E1 | NO: 49 | pairs | FKRKAGTSSLTECVLNKATNVAHWTTPSLKC |
| | | with | IRSGGSGGGGSGGGSGGGGSLQNWVNVISD |
| | | MH2 | LKKIEDLIQSMHIDATLYTESDVHPSCKVTAM |
| | | (SEQ ID | KCFLLELQVISLESGDASIHDTVENLIILANNS |
| | | NO: 50) | LSSNGNVTESGCKECEELEEKNIKEFLQSFVH |
| | | | IVQMFINTSGGGSIPVSLRSGGGGSSGGSGGS |
| | | | GGAVNGTSQFTCFYNSRANISCVWSQDGAL |
| | | | QDTSCQVHAWPDRRRWNQTCELLPVSQAS |
| | | | WACNLILGAPDSQKLTTVDIVTLRVLCREGV |
| | | | RWRVMAIQDFKPFENLRLMAPISLQVVHVET |
| | | | HRCNISWEISQASHYFERHLEFEARTLSPGHT |
| | | | WEEAPLLTLKQKQEWICLETLTPDTQYEFQV |
| | | | RVKPLQGEFTTWSPWSQPLAFRTKPAALGKD |
| | | | EPKSSDKTHTCPPCPAPELLGGPSVFLFPPKP |
| | | | KDTLMISRTPEVTCVVVDVSHEDPEVKFNW |
| | | | YVDGVEVHNAKTKPREEQYNSTYRVVSVLT |
| | | | VLHQDWLNGKEYKCKVSNKALPAPIEKTISK |
| | | | AKGQPREPQVYTLPPCRDELTKNQVSLWCLV |
| | | | KGFYPSDIAVEWESNGQPENNYKTTPPVLDS |
| | | | DGSFFLYSKLTVDKSRWQQGNVFSCSVMHE |
| | | | ALHNHYTQKSLSLSPGK |
| | SEQ ID | MH2 | EPKSSDKTHTCPPCPAPELLGGPSVFLFPPKP |
| | NO: 50 | | KDTLMISRTPEVTCVVVDVSHEDPEVKFNW |
| | | | YVDGVEVHNAKTKPREEQYNSTYRVVSVLT |
| | | | VLHQDWLNGKEYKCKVSNKALPAPIEKTISK |
| | | | AKGQPREPQVCTLPPSRDELTKNQVSLSCAV |
| | | | KGFYPSDIAVEWESNGQPENNYKTTPPVLDS |
| | | | DGSFFLVSKLTVDKSRWQQGNVFSCSVMHE |
| | | | ALHNHYTQKSLSLSPGK |
| Construct | SEQ ID | MK114- | ITCPPPMSVEHADIWVKSYSLYSRERYICNSG |
| E3 | NO: 51 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILANNSLSSNGNVTESGCKECEELE |
| | | | EKNIKEFLQSFVHIVQMFINTSGGGSIPVSLRS |
| | | | GGGGSSGGSGGSGGGTSQFTCFYNSRAQISC |
| | | | VWSQDGALQDTSCQVHAWPDRRRWNQTCE |
| | | | LLPVSQASWACNLILGAPDSQKLTTVDIVTLR |
| | | | VLCREGVRWRVMAIQDFKPFENLRLMAPISL |
| | | | QVVHVETHRAQISWEISQASHYFERHLEFEA |
| | | | RTLSPGHTWEEAPLLTLKQKQEWISLETLTPD |
| | | | TQYEFQVRVKPLQGEFTTWSPWSQPLAFRTK |
| | | | PAGGGGSGGGGSDKTHTCPPCPAPELLGGPS |
| | | | VFLFPPKPKDTLMISRTPEVTCVVVDVSHEDP |
| | | | EVKFNWYVDGVEVHNAKTKPREEQYNSTY |
| | | | RVVSVLTVLHQDWLNGKEYKCKVSNKALPA |
| | | | PIEKTISKAKGQPREPQVYTLPPCRDELTKNQ |
| | | | VSLWCLVKGFYPSDIAVEWESNGQPENNYKT |
| | | | TPPVLDSDGSFFLYSKLTVDKSRWQQGNVFS |
| | | | CSVMHEALHNHYTQKSLSLSPGK |
| | SEQ ID | MH7 | DKTHTCPPCPAPELLGGPSVFLFPPKPKDTLM |
| | NO: 52 | | ISRTPEVTCVVVDVSHEDPEVKFNWYVDGV |
| | | | EVHNAKTKPREEQYNSTYRVVSVLTVLHQD |
| | | | WLNGKEYKCKVSNKALPAPIEKTISKAKGQP |
| | | | REPQVCTLPPSRDELTKNQVSLSCAVKGFYPS |
| | | | DIAVEWESNGQPENNYKTTPPVLDSDGSFFL |
| | | | VSKLTVDKSRWQQGNVFSCSVMHEALHNH |
| | | | YTQKSLSLSPGK |
| | SEQ ID | MK115 | ITCPPPMSVEHADIWVKSYSLYSRERYICNSG |
| | NO: 53 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILAQNSLSSNGNVTESGCKECEELE |
| | | | EKNIKEFLQSFVHIVQMFIQTSGGGSIPVSLRS |
| | | | GGGGSSGGSGGSGGGTSQFTCFYNSRAQISC |
| | | | VWSQDGALQDTSCQVHAWPDRRRWNQTCE |
| | | | LLPVSQASWACNLILGAPDSQKLTTVDIVTLR |
| | | | VLCREGVRWRVMAIQDFKPFENLRLMAPISL |
| | | | QVVHVETHRAQISWEISQASHYFERHLEFEA |
| | | | RTLSPGHTWEEAPLLTLKQKQEWISLETLTPD |
| | | | TQYEFQVRVKPLQGEFTTWSPWSQPLAFRTK |
| | | | PAGGGGSGGGGSDKTHTCPPCPAPELLGGPS |
| | | | VFLFPPKPKDTLMISRTPEVTCVVVDVSHEDP |
| | | | EVKFNWYVDGVEVHNAKTKPREEQYNSTY |
| | | | RVVSVLTVLHQDWLNGKEYKCKVSNKALPA |
| | | | PIEKTISKAKGQPREPQVYTLPPCRDELTKNQ |
| | | | VSLWCLVKGFYPSDIAVEWESNGQPENNYKT |
| | | | TPPVLDSDGSFFLYSKLTVDKSRWQQGNVFS |
| | | | CSVMHEALHNHYTQKSLSLSPGK |
| | SEQ ID | MK117 | ITCPPPMSVEHADIWVKSYSLYSRERYICNSG |
| | NO: 54 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILANNSLSSNGNVTESGCKECEELE |
| | | | EKNIKEFLQSFVHIVQMFINTSGGGSIPVSLRS |
| | | | GGGGSSGGSGGSGGAVNGTSQFTCFYNSRA |
| | | | NISCVWSQDGALQDTSCQVHAWPDRRRWN |
| | | | QTCELLPVSQASWACNLILGAPDSQKLTTVDI |
| | | | VTLRVLCREGVRWRVMAIQDFKPFENLRLM |
| | | | APISLQVVHVETHRANISWEISQASHYFERHL |
| | | | EFEARTLSPGHTWEEAPLLTLKQKQEWITLE |
| | | | TLTPDTQYEFQVRVKPLQGEFTTWSPWSQPL |
| | | | AFRTKPAALGKDTGGGGSEPKSSDKTHTCPP |
| | | | CPAPELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEYK |
| | | | CKVSNKALPAPIEKTISKAKGQPREPQVYTLP |
| | | | PCRDELTKNQVSLWCLVKGFYPSDIAVEWES |
| | | | NGQPENNYKTTPPVLDSDGSFFLYSKLTVDK |
| | | | SRWQQGNVFSCSVMHEALHNHYTQKSLSLS |
| | | | PGK |
| | SEQ ID | MK118 | ITCPPPMSVEHADIWVKSYSLYSRERYICNSG |
| | NO: 55 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILANNSLSSNGNVTESGCKECEELE |
| | | | EKNIKEFLQSFVHIVQMFINTSGGGSIPVSLRS |
| | | | GGGGSSGGSGGSGGAVQGTSQFTCFYNSRA |
| | | | QISCVWSQDGALQDTSCQVHAWPDRRRWN |
| | | | QTCELLPVSQASWACNLILGAPDSQKLTTVDI |
| | | | VTLRVLCREGVRWRVMAIQDFKPFENLRLM |
| | | | APISLQVVHVETHRANISWEISQASHYFERHL |
| | | | EFEARTLSPGHTWEEAPLLTLKQKQEWITLE |
| | | | TLTPDTQYEFQVRVKPLQGEFTTWSPWSQPL |
| | | | AFRTKPAALGKDTGGGGSEPKSSDKTHTCPP |
| | | | CPAPELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEYK |
| | | | CKVSNKALPAPIEKTISKAKGQPREPQVYTLP |
| | | | PCRDELTKNQVSLWCLVKGFYPSDIAVEWES |
| | | | NGQPENNYKTTPPVLDSDGSFFLYSKLTVDK |
| | | | SRWQQGNVFSCSVMHEALHNHYTQKSLSLS |
| | | | PGK |
| | SEQ ID | MK119 | ITCPPPMSVEHADIWVKSYSLYSRERYICNSG |
| | NO: 56 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILAQNSLSSNGNVTESGCKECEELE |
| | | | EKNIKEFLQSFVHIVQMFIQTSGGGSIPVSLRS |
| | | | GGGGSSGGSGGSGGAVQGTSQFTCFYNSRA |
| | | | QISCVWSQDGALQDTSCQVHAWPDRRRWN |
| | | | QTCELLPVSQASWACNLILGAPDSQKLTTVDI |
| | | | VTLRVLCREGVRWRVMAIQDFKPFENLRLM |
| | | | APISLQVVHVETHRANISWEISQASHYFERHL |
| | | | EFEARTLSPGHTWEEAPLLTLKQKQEWITLE |
| | | | TLTPDTQYEFQVRVKPLQGEFTTWSPWSQPL |
| | | | AFRTKPAALGKDTGGGGSEPKSSDKTHTCPP |
| | | | CPAPELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEYK |
| | | | CKVSNKALPAPIEKTISKAKGQPREPQVYTLP |
| | | | PCRDELTKNQVSLWCLVKGFYPSDIAVEWES |
| | | | NGQPENNYKTTPPVLDSDGSFFLYSKLTVDK |
| | | | SRWQQGNVFSCSVMHEALHNHYTQKSLSLS |
| | | | PGK |
| | SEQ ID | MK120 | ITCPPPMSVEHADIWVKSYSLYSRERYICNSG |
| | NO: 57 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILAQNSLSSNGNVTESGCKECEELE |
| | | | EKNIKEFLQSFVHIVQMFIQTSGGGSIPVSLRS |
| | | | GGGGSSGGSGGSGGAVQGTSQFTCFYNSRA |
| | | | QISCVWSQDGALQDTSCQVHAWPDRRRWN |
| | | | QTCELLPVSQASWACNLILGAPDSQKLTTVDI |
| | | | VTLRVLCREGVRWRVMAIQDFKPFENLRLM |
| | | | APISLQVVHVETHRAQISWEISQASHYFERHL |
| | | | EFEARTLSPGHTWEEAPLLTLKQKQEWITLE |
| | | | TLTPDTQYEFQVRVKPLQGEFTTWSPWSQPL |
| | | | AFRTKPAALGKDTGGGGSEPKSSDKTHTCPP |
| | | | CPAPELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEYK |
| | | | CKVSNKALPAPIEKTISKAKGQPREPQVYTLP |
| | | | PCRDELTKNQVSLWCLVKGFYPSDIAVEWES |
| | | | NGQPENNYKTTPPVLDSDGSFFLYSKLTVDK |
| | | | SRWQQGNVFSCSVMHEALHNHYTQKSLSLS |
| | | | PGK |
| | SEQ ID | MK121 | ITCPPPMSVEHADIWVKSYSLYSRERYICNSG |
| | NO: 58 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILANNSLSSNGNVTESGCKECEELE |
| | | | EKNIKEFLQSFVHIVQMFINTSGGGSVPLSLY |
| | | | SGRSASGGSGGGGSGSGAVNGTSQFTCFYNS |
| | | | RANISCVWSQDGALQDTSCQVHAWPDRRR |
| | | | WNQTCELLPVSQASWACNLILGAPDSQKLTT |
| | | | VDIVTLRVLCREGVRWRVMAIQDFKPFENLR |
| | | | LMAPISLQVVHVETHRANISWEISQASHYFE |
| | | | RHLEFEARTLSPGHTWEEAPLLTLKQKQEWI |
| | | | TLETLTPDTQYEFQVRVKPLQGEFTTWSPWS |
| | | | QPLAFRTKPAALGKDTGGGGSEPKSSDKTHT |
| | | | CPPCPAPELLGGPSVFLFPPKPKDTLMISRTPE |
| | | | VTCVVVDVSHEDPEVKFNWYVDGVEVHNA |
| | | | KTKPREEQYNSTYRVVSVLTVLHQDWLNGK |
| | | | EYKCKVSNKALPAPIEKTISKAKGQPREPQV |
| | | | YTLPPCRDELTKNQVSLWCLVKGFYPSDIAV |
| | | | EWESNGQPENNYKTTPPVLDSDGSFFLYSKL |
| | | | TVDKSRWQQGNVFSCSVMHEALHNHYTQK |
| | | | SLSLSPGK |
| | SEQ ID | MK123 | ITCPPPMSVEHADIWVKSYSLYSRERYICNSG |
| | NO: 59 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILANNSLSSNGNVTESGCKECEELE |
| | | | EKNIKEFLQSFVHIVQMFINTSGGGSIPVSLRS |
| | | | GGGGSSGGSGGSGGAVNGTSQFTCFYNSRA |
| | | | NISCVWSQDGALQDTSCQVHAWPDRRRWN |
| | | | QTCELLPVSQASWACNLILGAPDSQKLTTVDI |
| | | | VTLRVLCREGVRWRVMAIQDFKPFENLRLM |
| | | | APISLQVVHVETHRCNISWEISQASHYFERHL |
| | | | EFEARTLSPGHTWEEAPLLTLKQKQEWICLE |
| | | | TLTPDTQYEFQVRVKPLQGEFTTWSPWSQPL |
| | | | AFRTKPAALGKDTGGGGSEPKSSDKTHTCPP |
| | | | CPAPELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEYK |
| | | | CKVSNKALPAPIEKTISKAKGQPREPQVYTLP |
| | | | PCRDELTKNQVSLWCLVKGFYPSDIAVEWES |
| | | | NGQPENNYKTTPPVLDSDGSFFLYSKLTVDK |
| | | | SRWQQGNVFSCSVMHEALHNHYTQKSLSLS |
| | | | PGK |
| | SEQ ID | MK124 | ITCPPPMSVEHADIWVKSYSLYSRERYICNSG |
| | NO: 60 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILANNSLSSNGNVTESGCKECEELE |
| | | | EKNIKEFLQSFVHIVQMFINTSGGGSVPLSLY |
| | | | SGRSASGGSGGGGSGSGAVNGTSQFTCFYNS |
| | | | RANISCVWSQDGALQDTSCQVHAWPDRRR |
| | | | WNQTCELLPVSQASWACNLILGAPDSQKLTT |
| | | | VDIVTLRVLCREGVRWRVMAIQDFKPFENLR |
| | | | LMAPISLQVVHVETHRCNISWEISQASHYFE |
| | | | RHLEFEARTLSPGHTWEEAPLLTLKQKQEWI |
| | | | CLETLTPDTQYEFQVRVKPLQGEFTTWSPWS |
| | | | QPLAFRTKPAALGKDTGGGGSEPKSSDKTHT |
| | | | CPPCPAPELLGGPSVFLFPPKPKDTLMISRTPE |
| | | | VTCVVVDVSHEDPEVKFNWYVDGVEVHNA |
| | | | KTKPREEQYNSTYRVVSVLTVLHQDWLNGK |
| | | | EYKCKVSNKALPAPIEKTISKAKGQPREPQV |
| | | | YTLPPCRDELTKNQVSLWCLVKGFYPSDIAV |
| | | | EWESNGQPENNYKTTPPVLDSDGSFFLYSKL |
| | | | TVDKSRWQQGNVFSCSVMHEALHNHYTQK |
| | | | SLSLSPGK |
| | SEQ ID | MK125 | ITCPPPMSVEHADIWVKSYSLYSRERYICNSG |
| | NO: 61 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILANNSLSSNGNVTESGCKECEELE |
| | | | EKNIKEFLQSFVHIVQMFINTSGGGSIPVSLRS |
| | | | GGGGSSGGSGGSGGAVNGTSQFTCFYNSRA |
| | | | NISCVWSQDGALQDTSCQVHAWPDRRRWN |
| | | | QTCELLPVSQASWACNLILGAPDSQKLTTVDI |
| | | | VTLRVLCREGVRWRVMAIQDFKPFENLRLM |
| | | | APISLQVVHVETHRANISWEISQASHYFERHL |
| | | | EFEARTLSPGHTWEEAPLLTLKQKQEWITLE |
| | | | TLTPDTQYEFQVRVKPLQGEFTTWSPWSQPL |
| | | | AFRTKPAALGKDTEAAAKEPKSSDKTHTCPP |
| | | | CPAPELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEYK |
| | | | CKVSNKALPAPIEKTISKAKGQPREPQVYTLP |
| | | | PCRDELTKNQVSLWCLVKGFYPSDIAVEWES |
| | | | NGQPENNYKTTPPVLDSDGSFFLYSKLTVDK |
| | | | SRWQQGNVFSCSVMHEALHNHYTQKSLSLS |
| | | | PGK |
| | SEQ ID | MK126 | ITCPPPMSVEHADIWVKSYSLYSRERYICNSG |
| | NO: 62 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILANNSLSSNGNVTESGCKECEELE |
| | | | EKNIKEFLQSFVHIVQMFINTSGGGSVPLSLY |
| | | | SGRSASGGSGGGGSGSGAVNGTSQFTCFYNS |
| | | | RANISCVWSQDGALQDTSCQVHAWPDRRR |
| | | | WNQTCELLPVSQASWACNLILGAPDSQKLTT |
| | | | VDIVTLRVLCREGVRWRVMAIQDFKPFENLR |
| | | | LMAPISLQVVHVETHRANISWEISQASHYFE |
| | | | RHLEFEARTLSPGHTWEEAPLLTLKQKQEWI |
| | | | TLETLTPDTQYEFQVRVKPLQGEFTTWSPWS |
| | | | QPLAFRTKPAALGKDTAPAPAPEPKSSDKTHT |
| | | | CPPCPAPELLGGPSVFLFPPKPKDTLMISRTPE |
| | | | VTCVVVDVSHEDPEVKFNWYVDGVEVHNA |
| | | | KTKPREEQYNSTYRVVSVLTVLHQDWLNGK |
| | | | EYKCKVSNKALPAPIEKTISKAKGQPREPQV |
| | | | YTLPPCRDELTKNQVSLWCLVKGFYPSDIAV |
| | | | EWESNGQPENNYKTTPPVLDSDGSFFLYSKL |
| | | | TVDKSRWQQGNVFSCSVMHEALHNHYTQK |
| | | | SLSLSPGK |
| | SEQ ID | MK157 | ITCPPPMSVEHADIWVKSYSLYSRERYICNSG |
| | NO: 172 | pairs | FKRKAGTSSLTECVLNKATNVAHWTTPSLKC |
| | | with | IRDPALVHQRPAPPSGGSGGGGSGGGSGGGG |
| | | MH8 | SLQNWVNVISDLKKIEDLIQSMHIDATLYTES |
| | | (SEQ ID | DVHPSCKVTAMKCFLLELQVISLESGDASIH |
| | | NO: 75): | DTVENLIILAQNSLSSNGNVTESGCKECEELE |
| | | | EKNIKEFLQSFVHIVQMFIQTSGGGSVPLSLY |
| | | | SGRSASGGSGGGGSGTSQFTCFYNSRAQISC |
| | | | VWSQDGALQDTSCQVHAWPDRRRWKQTCE |
| | | | LLPVSQASWACNLILGAPDSQKLTTVDIVTLR |
| | | | VLCREGVRWRVMAIQDFKPFENLRLMAPISL |
| | | | QVVHVETHRAQISWEISQASHYFERHLEFEA |
| | | | RTLSPGHTWEEAPLLTLKQKQEWISLETLTPD |
| | | | TQYEFQVRVKPLQGEFTTWSPWSQPLAFRTK |
| | | | PAGGGGSGGGGSDKTHTCPPCPAPELLGGPS |
| | | | VFLFPPKPKDTLMISRTPEVTCVVVDVSHEDP |
| | | | EVKFNWYVDGVEVHNAKTKPREEQYNSTY |
| | | | RVVSVLTVLHQDWLNGKEYKCKVSNKALPA |
| | | | PIEKTISKAKGQPREPQVYTLPPSRDELTKNQ |
| | | | VSLWCLVKGFYPSDIAVEWESNGQPENNYKT |
| | | | TPPVLDSDGSFFLYSKLTVDKSRWQQGNVFS |
| | | | CSVMHEALHNHYTQKSLSLSPGK |
| Construct | SEQ ID | M111 | DKTHTCPPCPAPELLGGPSVFLFPPKPKDTLM |
| E2 | NO: 63 | pairs | ISRTPEVTCVVVDVSHEDPEVKFNWYVDGV |
| | | with | EVHNAKTKPREEQYNSTYRVVSVLTVLHQD |
| | | MH2 | WLNGKEYKCKVSNKALPAPIEKTISKAKGQP |
| | | (SEQ ID | REPQVYTLPPCRDELTKNQVSLWCLVKGFYP |
| | | NO: 64) | SDIAVEWESNGQPENNYKTTPPVLDSDGSFF |
| | | | LYSKLTVDKSRWQQGNVFSCSVMHEALHNH |
| | | | YTQKSLSLSPGKGGGGSAVNGTSQFTCFYNS |
| | | | RANISCVWSQDGALQDTSCQVHAWPDRRR |
| | | | WNQTCELLPVSQASWACNLILGAPDSQKLTT |
| | | | VDIVTLRVLCREGVRWRVMAIQDFKPFENLR |
| | | | LMAPISLQVVHVETHRCNISWEISQASHYFE |
| | | | RHLEFEARTLSPGHTWEEAPLLTLKQKQEWI |
| | | | CLETLTPDTQYEFQVRVKPLQGEFTTWSPWS |
| | | | QPLAFRTKPAALGKDGGGSIPVSLRSGGGGS |
| | | | SGGSGGSGGITCPPPMSVEHADIWVKSYSLY |
| | | | SRERYICNSGFKRKAGTSSLTECVLNKATNVA |
| | | | HWTTPSLKCIRSGGSGGGGSGGGSGGGGSL |
| | | | QNWVNVISDLKKIEDLIQSMHIDATLYTESDV |
| | | | HPSCKVTAMKCFLLELQVISLESGDASIHDTV |
| | | | ENLIILANNSLSSNGNVTESGCKECEELEEKN |
| | | | IKEFLQSFVHIVQMFINTS |
| | SEQ ID | MH2 | EPKSSDKTHTCPPCPAPELLGGPSVFLFPPKP |
| | NO: 64 | | KDTLMISRTPEVTCVVVDVSHEDPEVKFNW |
| | | | YVDGVEVHNAKTKPREEQYNSTYRVVSVLT |
| | | | VLHQDWLNGKEYKCKVSNKALPAPIEKTISK |
| | | | AKGQPREPQVCTLPPSRDELTKNQVSLSCAV |
| | | | KGFYPSDIAVEWESNGQPENNYKTTPPVLDS |
| | | | DGSFFLVSKLTVDKSRWQQGNVFSCSVMHE |
| | | | ALHNHYTQKSLSLSPGK |
| | SEQ ID | M2001 | DKTHTCPPCPAPELLGGPSVFLFPPKPKDTLM |
| | NO: 65 | pairs | ISRTPEVTCVVVDVSHEDPEVKFNWYVDGV |
| | | with | EVHNAKTKPREEQYNSTYRVVSVLTVLHQD |
| | | MH7 | WLNGKEYKCKVSNKALPAPIEKTISKAKGQP |
| | | (SEQ ID | REPQVYTLPPCRDELTKNQVSLWCLVKGFYP |
| | | NO: 52) | SDIAVEWESNGQPENNYKTTPPVLDSDGSFF |
| | | | LYSKLTVDKSRWQQGNVFSCSVMHEALHNH |
| | | | YTQKSLSLSPGGGGGSAVNGTSQFTCFYNSR |
| | | | ANISCVWSQDGALQDTSCQVHAWPDRRRW |
| | | | NQTCELLPVSQASWACNLILGAPDSQKLTTV |
| | | | DIVTLRVLCREGVRWRVMAIQDFKPFENLRL |
| | | | MAPISLQVVHVETHRANISWEISQASHYFER |
| | | | HLEFEARTLSPGHTWEEAPLLTLKQKQEWIT |
| | | | LETLTPDTQYEFQVRVKPLQGEFTTWSPWSQ |
| | | | PLAFRTKPAALGKDTGGGSSGGSGGSGGIPV |
| | | | SLRSGGGGSITCPPPMSVEHADIWVKSYSLYS |
| | | | RERYICNSGFKRKAGTSSLTECVLNKATNVA |
| | | | HWTTPSLKCIRDPALVHQRPAPPSGGSGGGG |
| | | | SGGGSGGGGSLQNWVNVISDLKKIEDLIQSM |
| | | | HIDATLYTESDVHPSCKVTAMKCFLLELQVIS |
| | | | LESGDASIHDTVENLIILANNSLSSNGNVTES |
| | | | GCKECEELEEKNIKEFLQSFVHIVQMFINTS |
| | SEQ ID | M2002 | DKTHTCPPCPAPELLGGPSVFLFPPKPKDTLM |
| | NO: 66 | pairs | ISRTPEVTCVVVDVSHEDPEVKFNWYVDGV |
| | | with | EVHNAKTKPREEQYNSTYRVVSVLTVLHQD |
| | | MH7 | WLNGKEYKCKVSNKALPAPIEKTISKAKGQP |
| | | (SEQ ID | REPQVYTLPPCRDELTKNQVSLWCLVKGFYP |
| | | NO: 52) | SDIAVEWESNGQPENNYKTTPPVLDSDGSFF |
| | | | LYSKLTVDKSRWQQGNVFSCSVMHEALHNH |
| | | | YTQKSLSLSPGGGGGSAVQGTSQFTCFYNSR |
| | | | AQISCVWSQDGALQDTSCQVHAWPDRRRW |
| | | | NQTCELLPVSQASWACNLILGAPDSQKLTTV |
| | | | DIVTLRVLCREGVRWRVMAIQDFKPFENLRL |
| | | | MAPISLQVVHVETHRANISWEISQASHYFER |
| | | | HLEFEARTLSPGHTWEEAPLLTLKQKQEWIT |
| | | | LETLTPDTQYEFQVRVKPLQGEFTTWSPWSQ |
| | | | PLAFRTKPAALGKDTGGGSSGGSGGSGGIPV |
| | | | SLRSGGGGSITCPPPMSVEHADIWVKSYSLYS |
| | | | RERYICNSGFKRKAGTSSLTECVLNKATNVA |
| | | | HWTTPSLKCIRDPALVHQRPAPPSGGSGGGG |
| | | | SGGGSGGGGSLQNWVNVISDLKKIEDLIQSM |
| | | | HIDATLYTESDVHPSCKVTAMKCFLLELQVIS |
| | | | LESGDASIHDTVENLIILAQNSLSSNGNVTES |
| | | | GCKECEELEEKNIKEFLQSFVHIVQMFIQTS |
| Construct | SEQ ID | MK136 | ITCPPPMSVEHADIWVKSYSLYSRERYICNSG |
| E3 | NO: 67 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILANNSLSSNGNVTESGCKECEELE |
| | | | EKNIKEFLQSFVHIVQMFINTSGGGSVPLSLY |
| | | | SGRSASGGSGGGGSGSGGTSQFTCFYNSRAQ |
| | | | ISCVWSQDGALQDTSCQVHAWPDRRRWNQ |
| | | | TCELLPVSQASWACNLILGAPDSQKLTTVDIV |
| | | | TLRVLCREGVRWRVMAIQDFKPFENLRLMA |
| | | | PISLQVVHVETHRAQISWEISQASHYFERHLE |
| | | | FEARTLSPGHTWEEAPLLTLKQKQEWISLETL |
| | | | TPDTQYEFQVRVKPLQGEFTTWSPWSQPLAF |
| | | | RTKPAGGGGSGGGGSDKTHTCPPCPAPELLG |
| | | | GPSVFLFPPKPKDTLMISRTPEVTCVVVDVSH |
| | | | EDPEVKFNWYVDGVEVHNAKTKPREEQYN |
| | | | STYRVVSVLTVLHQDWLNGKEYKCKVSNKA |
| | | | LPAPIEKTISKAKGQPREPQVYTLPPCRDELT |
| | | | KNQVSLWCLVKGFYPSDIAVEWESNGQPEN |
| | | | NYKTTPPVLDSDGSFFLYSKLTVDKSRWQQG |
| | | | NVFSCSVMHEALHNHYTQKSLSLSPGK |
| | SEQ ID | MK137 | ITCPPPMSVEHADIWVKSYSLYSRERYICNSG |
| | NO: 68 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILAQNSLSSNGNVTESGCKECEELE |
| | | | EKNIKEFLQSFVHIVQMFIQTSGGGSVPLSLY |
| | | | SGRSASGGSGGGGSGSGGTSQFTCFYNSRAQ |
| | | | ISCVWSQDGALQDTSCQVHAWPDRRRWNQ |
| | | | TCELLPVSQASWACNLILGAPDSQKLTTVDIV |
| | | | TLRVLCREGVRWRVMAIQDFKPFENLRLMA |
| | | | PISLQVVHVETHRAQISWEISQASHYFERHLE |
| | | | FEARTLSPGHTWEEAPLLTLKQKQEWISLETL |
| | | | TPDTQYEFQVRVKPLQGEFTTWSPWSQPLAF |
| | | | RTKPAGGGGSGGGGSDKTHTCPPCPAPELLG |
| | | | GPSVFLFPPKPKDTLMISRTPEVTCVVVDVSH |
| | | | EDPEVKFNWYVDGVEVHNAKTKPREEQYN |
| | | | STYRVVSVLTVLHQDWLNGKEYKCKVSNKA |
| | | | LPAPIEKTISKAKGQPREPQVYTLPPCRDELT |
| | | | KNQVSLWCLVKGFYPSDIAVEWESNGQPEN |
| | | | NYKTTPPVLDSDGSFFLYSKLTVDKSRWQQG |
| | | | NVFSCSVMHEALHNHYTQKSLSLSPGK |
| | SEQ ID | MK138 | ITCPPPMSVEHADIWVKSYSLYSRERYICNSG |
| | NO: 69 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILAQNSLSSNGNVTESGCKECEELE |
| | | | EKNIKEFLQSFVHIVQMFIQTSGGGSVPLSLY |
| | | | SGRSASGGSGGGGSGSGGTSQFTCFYNSRAQ |
| | | | ISCVWSQDGALQDTSCQVHAWPDRRRWNQ |
| | | | TCELLPVSQASWACNLILGAPDSQKLTTVDIV |
| | | | TLRVLCREGVRWRVMAIQDFKPFENLRLMA |
| | | | PISLQVVHVETHRAQISWEISQASHYFERHLE |
| | | | FEARTLSPGHTWEEAPLLTLKQKQEWISLETL |
| | | | TPDTQYEFQVRVKPLQGEFTTWSPWSQPLAF |
| | | | RTKPAGGGGSGGGGSDKTHTCPPCPAPELLG |
| | | | GPSVFLFPPKPKDTLMISRTPEVTCVVVDVSH |
| | | | EDPEVKFNWYVDGVEVHNAKTKPREEQYN |
| | | | STYRVVSVLTVLHQDWLNGKEYKCKVSNKA |
| | | | LPAPIEKTISKAKGQPREPQVYTLPPCRDELT |
| | | | KNQVSLWCLVKGFYPSDIAVEWESNGQPEN |
| | | | NYKTTPPVLDSDGSFFLYSKLTVDKSRWQQG |
| | | | NVFSCSVMHEALHNHYTQKSLSLSPGK |
| | SEQ ID | MK139 | ITCPPPMSVEHADIWVKSYSLYSRERYICNSG |
| | NO: 70 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILAQNSLSSNGNVTESGCKECEELE |
| | | | EKNIKEFLQSFVHIVQMFIQTSGGGSVPLSLY |
| | | | SGRSASGGSGGGGSGTSQFTCFYNSRAQISC |
| | | | VWSQDGALQDTSCQVHAWPDRRRWNQTCE |
| | | | LLPVSQASWACNLILGAPDSQKLTTVDIVTLR |
| | | | VLCREGVRWRVMAIQDFKPFENLRLMAPISL |
| | | | QVVHVETHRAQISWEISQASHYFERHLEFEA |
| | | | RTLSPGHTWEEAPLLTLKQKQEWISLETLTPD |
| | | | TQYEFQVRVKPLQGEFTTWSPWSQPLAFRTK |
| | | | PAGGGGSGGGGSDKTHTCPPCPAPELLGGPS |
| | | | VFLFPPKPKDTLMISRTPEVTCVVVDVSHEDP |
| | | | EVKFNWYVDGVEVHNAKTKPREEQYNSTY |
| | | | RVVSVLTVLHQDWLNGKEYKCKVSNKALPA |
| | | | PIEKTISKAKGQPREPQVYTLPPCRDELTKNQ |
| | | | VSLWCLVKGFYPSDIAVEWESNGQPENNYKT |
| | | | TPPVLDSDGSFFLYSKLTVDKSRWQQGNVFS |
| | | | CSVMHEALHNHYTQKSLSLSPGK |
| | SEQ ID | MK140 | ITCPPPMSVEHADIWVKSYSLYSRERYICNSG |
| | NO: 71 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILAQNSLSSNGNVTESGCKECEELE |
| | | | EKNIKEFLQSFVHIVQMFIQTSGGGSVPLSLY |
| | | | SGRSASGGSGGGTSQFTCFYNSRAQISCVWS |
| | | | QDGALQDTSCQVHAWPDRRRWNQTCELLP |
| | | | VSQASWACNLILGAPDSQKLTTVDIVTLRVL |
| | | | CREGVRWRVMAIQDFKPFENLRLMAPISLQV |
| | | | VHVETHRAQISWEISQASHYFERHLEFEART |
| | | | LSPGHTWEEAPLLTLKQKQEWISLETLTPDTQ |
| | | | YEFQVRVKPLQGEFTTWSPWSQPLAFRTKPA |
| | | | GGGGSGGGGSDKTHTCPPCPAPELLGGPSVF |
| | | | LFPPKPKDTLMISRTPEVTCVVVDVSHEDPE |
| | | | VKFNWYVDGVEVHNAKTKPREEQYNSTYR |
| | | | VVSVLTVLHQDWLNGKEYKCKVSNKALPAP |
| | | | IEKTISKAKGQPREPQVYTLPPCRDELTKNQV |
| | | | SLWCLVKGFYPSDIAVEWESNGQPENNYKTT |
| | | | PPVLDSDGSFFLYSKLTVDKSRWQQGNVFSC |
| | | | SVMHEALHNHYTQKSLSLSPGK |
| | SEQ ID | MK141 | ITCPPPMSVEHADIWVKSYSLYSRERYICNSG |
| | NO: 72 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILAQNSLSSNGNVTESGCKECEELE |
| | | | EKNIKEFLQSFVHIVQMFIQTSGGGSVPLSLY |
| | | | SGRSASGGSGGGGSGSGGTSQFTCFYNSRAQ |
| | | | ISCVWSQDGALQDTSCQVHAWPDRRRWNQ |
| | | | TCELLPVSQASWACNLILGAPDSQKLTTQDIV |
| | | | TLRVLCREGVRWRVMAIQDFKPFENLRLMA |
| | | | PISLQVVHVETHRAQISWEISQASHYFERHLE |
| | | | FEARTLSPGHTWEEAPLLTLKQKQEWISLETL |
| | | | TPDTQYEFQVRVKPLQGEFTTWSPWSQPLAF |
| | | | RTKPAGGGGSGGGGSDKTHTCPPCPAPELLG |
| | | | GPSVFLFPPKPKDTLMISRTPEVTCVVVDVSH |
| | | | EDPEVKFNWYVDGVEVHNAKTKPREEQYN |
| | | | STYRVVSVLTVLHQDWLNGKEYKCKVSNKA |
| | | | LPAPIEKTISKAKGQPREPQVYTLPPCRDELT |
| | | | KNQVSLWCLVKGFYPSDIAVEWESNGQPEN |
| | | | NYKTTPPVLDSDGSFFLYSKLTVDKSRWQQG |
| | | | NVFSCSVMHEALHNHYTQKSLSLSPGK |
| | SEQ ID | MK146 | ITCPPPMSVEHADIWVKSYSLYSRERYICNSG |
| | NO: 73 | pairs | FKRKAGTSSLTECVLNKATNVAHWTTPSLKC |
| | | with | IRDPALVHQRPAPPSGGGSGGGGSGGGGSGG |
| | | MH7 | GGSGGGSLQNWVNVISDLKKIEDLIQSMHID |
| | | (SEQ ID | ATLYTESDVHPSCKVTAMKCFLLELQVISLES |
| | | NO: 52) | GDASIHDTVENLIILAQNSLSSNGNVTESGCK |
| | | | ECEELEEKNIKEFLQSFVHIVQMFIQTSGGGS |
| | | | VPLSLYSGRSASGGSGGGGSGSGGTSQFTCF |
| | | | YNSRAQISCVWSQDGALQDTSCQVHAWPDR |
| | | | RRWNQTCELLPVSQASWACNLILGAPDSQKL |
| | | | TTVDIVTLRVLCREGVRWRVMAIQDFKPFEN |
| | | | LRLMAPISLQVVHVETHRAQISWEISQASHY |
| | | | FERHLEFEARTLSPGHTWEEAPLLTLKQKQE |
| | | | WISLETLTPDTQYEFQVRVKPLQGEFTTWSP |
| | | | WSQPLAFRTKPAGGGGSGGGGSDKTHTCPP |
| | | | CPAPELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEYK |
| | | | CKVSNKALPAPIEKTISKAKGQPREPQVYTLP |
| | | | PSRDELTKNQVSLWCLVKGFYPSDIAVEWES |
| | | | NGQPENNYKTTPPVLDSDGSFFLYSKLTVDK |
| | | | SRWQQGNVFSCSVMHEALHNHYTQKSLSLS |
| | | | PGK |
| | SEQ ID | MK145 | ITCPPPMSVEHADIWVKSYSLYSRERYICNSG |
| | NO: 74 | pairs | FKRKAGTSSLTECVLNKATNVAHWTTPSLKC |
| | | with | IRDPALVHQRPAPPSGGSGGGGSGGGSGGGG |
| | | MH8 | SLQNWVNVISDLKKIEDLIQSMHIDATLYTES |
| | | (SEQ ID | DVHPSCKVTAMKCFLLELQVISLESGDASIH |
| | | NO: 75) | DTVENLIILAQNSLSSNGNVTESGCKECEELE |
| | | | EKNIKEFLQSFVHIVQMFIQTSGGGSVPLSLY |
| | | | SGRSASGGSGGGGSGSGGTSQFTCFYNSRAQ |
| | | | ISCVWSQDGALQDTSCQVHAWPDRRRWNQ |
| | | | TCELLPVSQASWACNLILGAPDSQKLTTVDIV |
| | | | TLRVLCREGVRWRVMAIQDFKPFENLRLMA |
| | | | PISLQVVHVETHRAQISWEISQASHYFERHLE |
| | | | FEARTLSPGHTWEEAPLLTLKQKQEWISLETL |
| | | | TPDTQYEFQVRVKPLQGEFTTWSPWSQPLAF |
| | | | RTKPAGGGGSGGGGSDKTHTCPPCPAPELLG |
| | | | GPSVFLFPPKPKDTLMISRTPEVTCVVVDVSH |
| | | | EDPEVKFNWYVDGVEVHNAKTKPREEQYN |
| | | | STYRVVSVLTVLHQDWLNGKEYKCKVSNKA |
| | | | LPAPIEKTISKAKGQPREPQVYTLPPSRDELTK |
| | | | NQVSLWCLVKGFYPSDIAVEWESNGQPENN |
| | | | YKTTPPVLDSDGSFFLYSKLTVDKSRWQQGN |
| | | | VFSCSVMHEALHNHYTQKSLSLSPGK |
| | SEQ ID | MH8 | DKTHTCPPCPAPELLGGPSVFLFPPKPKDTLM |
| | NO: 75 | | ISRTPEVTCVVVDVSHEDPEVKFNWYVDGV |
| | | | EVHNAKTKPREEQYNSTYRVVSVLTVLHQD |
| | | | WLNGKEYKCKVSNKALPAPIEKTISKAKGQP |
| | | | REPQVYTLPPSRDELTKNQVSLSCAVKGFYPS |
| | | | DIAVEWESNGQPENNYKTTPPVLDSDGSFFL |
| | | | VSKLTVDKSRWQQGNVFSCSVMHEALHNH |
| | | | YTQKSLSLSPGK |
| | SEQ ID | MK149 | ITCPPPMSVEHADIWVKSYSLYSRERYICNSG |
| | NO: 76 | pairs | FKRKAGTSSLTECVLNKATNVAHWTTPSLKC |
| | | with | IRDPALVHQRPAPPSGGSGGGGSGGGSGGGG |
| | | MH8 | SLQNWVNVISDLKKIEDLIQSMHIDATLYTES |
| | | (SEQ ID | DVHPSCKVTAMKCFLLELQVISLESGDASIH |
| | | NO: 75) | DTVENLIILAQNSLSSNGNVTESGCKECEELE |
| | | | EKNIKEFLQSFVHIVQMFIQTSGGGSVPLSLY |
| | | | SGRSASGGSGGGGSGTSQFTCFYNSRAQISC |
| | | | VWSQDGALQDTSCQVHAWPDRRRWNQTCE |
| | | | LLPVSQASWACNLILGAPDSQKLTTVDIVTLR |
| | | | VLCREGVRWRVMAIQDFKPFENLRLMAPISL |
| | | | QVVHVETHRAQISWEISQASHYFERHLEFEA |
| | | | RTLSPGHTWEEAPLLTLKQKQEWISLETLTPD |
| | | | TQYEFQVRVKPLQGEFTTWSPWSQPLAFRTK |
| | | | PAGGGGSGGGGSDKTHTCPPCPAPELLGGPS |
| | | | VFLFPPKPKDTLMISRTPEVTCVVVDVSHEDP |
| | | | EVKFNWYVDGVEVHNAKTKPREEQYNSTY |
| | | | RVVSVLTVLHQDWLNGKEYKCKVSNKALPA |
| | | | PIEKTISKAKGQPREPQVYTLPPSRDELTKNQ |
| | | | VSLWCLVKGFYPSDIAVEWESNGQPENNYKT |
| | | | TPPVLDSDGSFFLYSKLTVDKSRWQQGNVFS |
| | | | CSVMHEALHNHYTQKSLSLSPGK |
| | SEQ ID | MK150 | ITCPPPMSVEHADIWVKSYSLYSRERYICNSG |
| | NO: 77 | pairs | FKRKAGTSSLTECVLNKATNVAHWTTPSLKC |
| | | with | IRDPALVHQRPAPPSGGSGGGGSGGGSGGGG |
| | | MH8 | SLQNWVNVISDLKKIEDLIQSMHIDATLYTES |
| | | (SEQ ID | DVHPSCKVTAMKCFLLELQVISLESGDASIH |
| | | NO: 75) | DTVENLIILAQNSLSSNGQVTESGCKECEELE |
| | | | EKNIKEFLQSFVHIVQMFIQTSGGGSVPLSLY |
| | | | SGRSASGGSGGGGSGTSQFTCFYNSRAQISC |
| | | | VWSQDGALQDTSCQVHAWPDRRRWNQTCE |
| | | | LLPVSQASWACNLILGAPDSQKLTTVDIVTLR |
| | | | VLCREGVRWRVMAIQDFKPFENLRLMAPISL |
| | | | QVVHVETHRAQISWEISQASHYFERHLEFEA |
| | | | RTLSPGHTWEEAPLLTLKQKQEWISLETLTPD |
| | | | TQYEFQVRVKPLQGEFTTWSPWSQPLAFRTK |
| | | | PAGGGGSGGGGSDKTHTCPPCPAPELLGGPS |
| | | | VFLFPPKPKDTLMISRTPEVTCVVVDVSHEDP |
| | | | EVKFNWYVDGVEVHNAKTKPREEQYNSTY |
| | | | RVVSVLTVLHQDWLNGKEYKCKVSNKALPA |
| | | | PIEKTISKAKGQPREPQVYTLPPSRDELTKNQ |
| | | | VSLWCLVKGFYPSDIAVEWESNGQPENNYKT |
| | | | TPPVLDSDGSFFLYSKLTVDKSRWQQGNVFS |
| | | | CSVMHEALHNHYTQKSLSLSPGK |
| | SEQ ID | MK151 | ITCPPPMSVEHADIWVKSYSLYSRERYICNSG |
| | NO: 78 | pairs | FKRKAGTSSLTECVLNKATNVAHWTTPSLKC |
| | | with | IRDPALVHQRPAPPSGGSGGGGSGGGSGGGG |
| | | MH8 | SLQNWVNVISDLKKIEDLIQSMHIDATLYTES |
| | | (SEQ ID | DVHPSCKVTAMKCFLLELQVISLESGDASIH |
| | | NO: 75) | DTVENLIILAQNSLSSNGQVTESGCKECEELE |
| | | | EKNIKEFLQSFVHIVQMFIQTSGGGSVPLSLY |
| | | | SGRSASGGSGGGGSGTSQFTCFYNSRAQISC |
| | | | VWSQDGALQDTSCQVHAWPDRRRWKQTCE |
| | | | LLPVSQASWACNLILGAPDSQKLTTVDIVTLR |
| | | | VLCREGVRWRVMAIQDFKPFENLRLMAPISL |
| | | | QVVHVETHRAQISWEISQASHYFERHLEFEA |
| | | | RTLSPGHTWEEAPLLTLKQKQEWISLETLTPD |
| | | | TQYEFQVRVKPLQGEFTTWSPWSQPLAFRTK |
| | | | PAGGGGSGGGGSDKTHTCPPCPAPELLGGPS |
| | | | VFLFPPKPKDTLMISRTPEVTCVVVDVSHEDP |
| | | | EVKFNWYVDGVEVHNAKTKPREEQYNSTY |
| | | | RVVSVLTVLHQDWLNGKEYKCKVSNKALPA |
| | | | PIEKTISKAKGQPREPQVYTLPPSRDELTKNQ |
| | | | VSLWCLVKGFYPSDIAVEWESNGQPENNYKT |
| | | | TPPVLDSDGSFFLYSKLTVDKSRWQQGNVFS |
| | | | CSVMHEALHNHYTQKSLSLSPGK |
| | SEQ ID | MK152 | ITCPPPMSVEHADIWVKSYSLYSRERYICNSG |
| | NO: 79 | pairs | FKRKAGTSSLTECVLNKATNVAHWTTPSLKC |
| | | with | IRDPALVHQRPAPPSGGSGGGGSGGGSGGGG |
| | | MH8 | SLQNWVNVISDLKKIEDLIQSMHIDATLYTES |
| | | (SEQ ID | DVHPSCKVTAMKCFLLELQVISLESGDASIH |
| | | NO: 75) | DTVENLIILAQNSLSSNGQVTESGCKECEELE |
| | | | EKNIKEFLQSFVHIVQMFIQTSGGGSVPLSLY |
| | | | SGRSASGGSGGGGSGTSQFTCFYNSRAQISC |
| | | | VWSQDGALQDTSCQVHAWPDRRRWRQTCE |
| | | | LLPVSQASWACNLILGAPDSQKLTTVDIVTLR |
| | | | VLCREGVRWRVMAIQDFKPFENLRLMAPISL |
| | | | QVVHVETHRAQISWEISQASHYFERHLEFEA |
| | | | RTLSPGHTWEEAPLLTLKQKQEWISLETLTPD |
| | | | TQYEFQVRVKPLQGEFTTWSPWSQPLAFRTK |
| | | | PAGGGGSGGGGSDKTHTCPPCPAPELLGGPS |
| | | | VFLFPPKPKDTLMISRTPEVTCVVVDVSHEDP |
| | | | EVKFNWYVDGVEVHNAKTKPREEQYNSTY |
| | | | RVVSVLTVLHQDWLNGKEYKCKVSNKALPA |
| | | | PIEKTISKAKGQPREPQVYTLPPSRDELTKNQ |
| | | | VSLWCLVKGFYPSDIAVEWESNGQPENNYKT |
| | | | TPPVLDSDGSFFLYSKLTVDKSRWQQGNVFS |
| | | | CSVMHEALHNHYTQKSLSLSPGK |
| | SEQ ID | MK153 | ITCPPPMSVEHADIWVKSYSLYSRERYICNSG |
| | NO: 80 | pairs | FKRKAGTSSLTECVLNKATNVAHWTTPSLKC |
| | | with | IRDPALVHQRPAPPSGGSGGGGSGGGSGGGG |
| | | MH8 | SLQNWVNVISDLKKIEDLIQSMHIDATLYTES |
| | | (SEQ ID | DVHPSCKVTAMKCFLLELQVISLESGDASIH |
| | | NO: 75) | DTVENLIILAQNSLSSNGQVTESGCKECEELE |
| | | | EKNIKEFLQSFVHIVQMFIQTSGGGSVPLSLY |
| | | | SGRSASGGSGGGGSGTSQFTCFYNSRAQISC |
| | | | VWSQDGALQDTSCQVHAWPDRRRWQQTCE |
| | | | LLPVSQASWACNLILGAPDSQKLTTVDIVTLR |
| | | | VLCREGVRWRVMAIQDFKPFENLRLMAPISL |
| | | | QVVHVETHRAQISWEISQASHYFERHLEFEA |
| | | | RTLSPGHTWEEAPLLTLKQKQEWISLETLTPD |
| | | | TQYEFQVRVKPLQGEFTTWSPWSQPLAFRTK |
| | | | PAGGGGSGGGGSDKTHTCPPCPAPELLGGPS |
| | | | VFLFPPKPKDTLMISRTPEVTCVVVDVSHEDP |
| | | | EVKFNWYVDGVEVHNAKTKPREEQYNSTY |
| | | | RVVSVLTVLHQDWLNGKEYKCKVSNKALPA |
| | | | PIEKTISKAKGQPREPQVYTLPPSRDELTKNQ |
| | | | VSLWCLVKGFYPSDIAVEWESNGQPENNYKT |
| | | | TPPVLDSDGSFFLYSKLTVDKSRWQQGNVFS |
| | | | CSVMHEALHNHYTQKSLSLSPGK |
| | SEQ ID | MK154 | ITCPPPMSVEHADIWVKSYSLYSRERYICNSG |
| | NO: 81 | pairs | FKRKAGTSSLTECVLNKATNVAHWTTPSLKC |
| | | with | IRDPALVHQRPAPPSGGSGGGGSGGGSGGGG |
| | | MH8 | SLQNWVNVISDLKKIEDLIQSMHIDATLYTES |
| | | (SEQ ID | DVHPSCKVTAMKCFLLELQVISLESGDASIH |
| | | NO: 75) | DTVENLIILAQNSLSSNGQVTESGCKECEELE |
| | | | EKNIKEFLQSFVHIVQMFIQTSGGGSVPLSLY |
| | | | SGRSASGGSGGGGSGSGGTSQFTCFYNSRAQ |
| | | | ISCVWSQDGALQDTSCQVHAWPDRRRWNQ |
| | | | TCELLPVSQASWACNLILGAPDSQKLTTVDIV |
| | | | TLRVLCREGVRWRVMAIQDFKPFENLRLMA |
| | | | PISLQVVHVETHRAQISWEISQASHYFERHLE |
| | | | FEARTLSPGHTWEEAPLLTLKQKQEWISLETL |
| | | | TPDTQYEFQVRVKPLQGEFTTWSPWSQPLAF |
| | | | RTKPAGGGGSGGGGSDKTHTCPPCPAPELLG |
| | | | GPSVFLFPPKPKDTLMISRTPEVTCVVVDVSH |
| | | | EDPEVKFNWYVDGVEVHNAKTKPREEQYN |
| | | | STYRVVSVLTVLHQDWLNGKEYKCKVSNKA |
| | | | LPAPIEKTISKAKGQPREPQVYTLPPSRDELTK |
| | | | NQVSLWCLVKGFYPSDIAVEWESNGQPENN |
| | | | YKTTPPVLDSDGSFFLYSKLTVDKSRWQQGN |
| | | | VFSCSVMHEALHNHYTQKSLSLSPGK |
| | SEQ ID | MK155 | ITCPPPMSVEHADIWVKSYSLYSRERYICNSG |
| | NO: 82 | pairs | FKRKAGTSSLTECVLNKATNVAHWTTPSLKC |
| | | with | IRDPALVHQRPAPPSGGSGGGGSGGGSGGGG |
| | | MH7 | SLQNWVNVISDLKKIEDLIQSMHIDATLYTES |
| | | (SEQ ID | DVHPSCKVTAMKCFLLELQVISLESGDASIH |
| | | NO: 52) | DTVENLIILAQNSLSSNGQVTESGCKECEELE |
| | | | EKNIKEFLQSFVHIVQMFIQTSGGGSVPLSLY |
| | | | SGRSASGGSGGGGSGTSQFTCFYNSRAQISC |
| | | | VWSQDGALQDTSCQVHAWPDRRRWNQTCE |
| | | | LLPVSQASWACNLILGAPDSQKLTTVDIVTLR |
| | | | VLCREGVRWRVMAIQDFKPFENLRLMAPISL |
| | | | QVVHVETHRAQISWEISQASHYFERHLEFEA |
| | | | RTLSPGHTWEEAPLLTLKQKQEWISLETLTPD |
| | | | TQYEFQVRVKPLQGEFTTWSPWSQPLAFRTK |
| | | | PAGGGGSGGGGSDKTHTCPPCPAPELLGGPS |
| | | | VFLFPPKPKDTLMISRTPEVTCVVVDVSHEDP |
| | | | EVKFNWYVDGVEVHNAKTKPREEQYNSTY |
| | | | RVVSVLTVLHQDWLNGKEYKCKVSNKALPA |
| | | | PIEKTISKAKGQPREPQVYTLPPCRDELTKNQ |
| | | | VSLWCLVKGFYPSDIAVEWESNGQPENNYKT |
| | | | TPPVLDSDGSFFLYSKLTVDKSRWQQGNVFS |
| | | | CSVMHEALHNHYTQKSLSLSPGK |
| Construct | SEQ ID | M109 | DKTHTCPPCPAPELLGGPSVFLFPPKPKDTLM |
| F1 | NO: 83 | pairs | ISRTPEVTCVVVDVSHEDPEVKFNWYVDGV |
| | | with | EVHNAKTKPREEQYNSTYRVVSVLTVLHQD |
| | | MH110 | WLNGKEYKCKVSNKALPAPIEKTISKAKGQP |
| | | (SEQ ID | REPQVYTLPPCRDELTKNQVSLWCLVKGFYP |
| | | NO: 84 | SDIAVEWESNGQPENNYKTTPPVLDSDGSFF |
| | | | LYSKLTVDKSRWQQGNVFSCSVMHEALHNH |
| | | | YTQKSLSLSPGKGGGSIPVSLRSGGGGSSGGS |
| | | | GGSGGITCPPPMSVEHADIWVKSYSLYSRER |
| | | | YICNSGFKRKAGTSSLTECVLNKATNVAHWT |
| | | | TPSLKCIRSGGSGGGGSGGGSGGGGSLQNW |
| | | | VNVISDLKKIEDLIQSMHIDATLYTESDVHPS |
| | | | CKVTAMKCFLLELQVISLESGDASIHDTVEN |
| | | | LIILANNSLSSNGNVTESGCKECEELEEKNIK |
| | | | EFLQSFVHIVQMFINTS |
| | SEQ ID | MH110 | DKTHTCPPCPAPELLGGPSVFLFPPKPKDTLM |
| | NO: 84 | | ISRTPEVTCVVVDVSHEDPEVKFNWYVDGV |
| | | | EVHNAKTKPREEQYNSTYRVVSVLTVLHQD |
| | | | WLNGKEYKCKVSNKALPAPIEKTISKAKGQP |
| | | | REPQVCTLPPSRDELTKNQVSLSCAVKGFYPS |
| | | | DIAVEWESNGQPENNYKTTPPVLDSDGSFFL |
| | | | VSKLTVDKSRWQQGNVFSCSVMHEALHNH |
| | | | YTQKSLSLSPGKGGGGSAVNGTSQFTCFYNS |
| | | | RANISCVWSQDGALQDTSCQVHAWPDRRR |
| | | | WNQTCELLPVSQASWACNLILGAPDSQKLTT |
| | | | VDIVTLRVLCREGVRWRVMAIQDFKPFENLR |
| | | | LMAPISLQVVHVETHRCNISWEISQASHYFE |
| | | | RHLEFEARTLSPGHTWEEAPLLTLKQKQEWI |
| | | | CLETLTPDTQYEFQVRVKPLQGEFTTWSPWS |
| | | | QPLAFRTKPAALGKD |
| | SEQ ID | M2003 | DKTHTCPPCPAPELLGGPSVFLFPPKPKDTLM |
| | NO: 85 | pairs | ISRTPEVTCVVVDVSHEDPEVKFNWYVDGV |
| | | with | EVHNAKTKPREEQYNSTYRVVSVLTVLHQD |
| | | MH2004 | WLNGKEYKCKVSNKALPAPIEKTISKAKGQP |
| | | (SEQ ID | REPQVYTLPPCRDELTKNQVSLWCLVKGFYP |
| | | NO: 86) | SDIAVEWESNGQPENNYKTTPPVLDSDGSFF |
| | | | LYSKLTVDKSRWQQGNVFSCSVMHEALHNH |
| | | | YTQKSLSLSPGGGGSIPVSLRSGGGGSITCPPP |
| | | | MSVEHADIWVKSYSLYSRERYICNSGFKRKA |
| | | | GTSSLTECVLNKATNVAHWTTPSLKCIRDPAL |
| | | | VHQRPAPPSGGSGGGGSGGGSGGGGSLQNW |
| | | | VNVISDLKKIEDLIQSMHIDATLYTESDVHPS |
| | | | CKVTAMKCFLLELQVISLESGDASIHDTVEN |
| | | | LIILANNSLSSNGNVTESGCKECEELEEKNIK |
| | | | EFLQSFVHIVQMFINTS |
| | SEQ ID | MH2004 | DKTHTCPPCPAPELLGGPSVFLFPPKPKDTLM |
| | NO: 86 | | ISRTPEVTCVVVDVSHEDPEVKFNWYVDGV |
| | | | EVHNAKTKPREEQYNSTYRVVSVLTVLHQD |
| | | | WLNGKEYKCKVSNKALPAPIEKTISKAKGQP |
| | | | REPQVCTLPPSRDELTKNQVSLSCAVKGFYPS |
| | | | DIAVEWESNGQPENNYKTTPPVLDSDGSFFL |
| | | | VSKLTVDKSRWQQGNVFSCSVMHEALHNH |
| | | | YTQKSLSLSPGGGGGSAVNGTSQFTCFYNSR |
| | | | ANISCVWSQDGALQDTSCQVHAWPDRRRW |
| | | | NQTCELLPVSQASWACNLILGAPDSQKLTTV |
| | | | DIVTLRVLCREGVRWRVMAIQDFKPFENLRL |
| | | | MAPISLQVVHVETHRANISWEISQASHYFER |
| | | | HLEFEARTLSPGHTWEEAPLLTLKQKQEWIT |
| | | | LETLTPDTQYEFQVRVKPLQGEFTTWSPWSQ |
| | | | PLAFRTKPAALGKDT |
| | SEQ ID | M2003 | DKTHTCPPCPAPELLGGPSVFLFPPKPKDTLM |
| | NO: 87 | pairs | ISRTPEVTCVVVDVSHEDPEVKFNWYVDGV |
| | | with | EVHNAKTKPREEQYNSTYRVVSVLTVLHQD |
| | | M2005 | WLNGKEYKCKVSNKALPAPIEKTISKAKGQP |
| | | (SEQ ID | REPQVYTLPPCRDELTKNQVSLWCLVKGFYP |
| | | NO: 88) | SDIAVEWESNGQPENNYKTTPPVLDSDGSFF |
| | | | LYSKLTVDKSRWQQGNVFSCSVMHEALHNH |
| | | | YTQKSLSLSPGGGGSIPVSLRSGGGGSITCPPP |
| | | | MSVEHADIWVKSYSLYSRERYICNSGFKRKA |
| | | | GTSSLTECVLNKATNVAHWTTPSLKCIRDPAL |
| | | | VHQRPAPPSGGSGGGGSGGGSGGGGSLQNW |
| | | | VNVISDLKKIEDLIQSMHIDATLYTESDVHPS |
| | | | CKVTAMKCFLLELQVISLESGDASIHDTVEN |
| | | | LIILANNSLSSNGNVTESGCKECEELEEKNIK |
| | | | EFLQSFVHIVQMFINTS |
| | SEQ ID | M2005 | DKTHTCPPCPAPELLGGPSVFLFPPKPKDTLM |
| | NO: 88 | | ISRTPEVTCVVVDVSHEDPEVKFNWYVDGV |
| | | | EVHNAKTKPREEQYNSTYRVVSVLTVLHQD |
| | | | WLNGKEYKCKVSNKALPAPIEKTISKAKGQP |
| | | | REPQVCTLPPSRDELTKNQVSLSCAVKGFYPS |
| | | | DIAVEWESNGQPENNYKTTPPVLDSDGSFFL |
| | | | VSKLTVDKSRWQQGNVFSCSVMHEALHNH |
| | | | YTQKSLSLSPGGGGGSAVQGTSQFTCFYNSR |
| | | | AQISCVWSQDGALQDTSCQVHAWPDRRRW |
| | | | NQTCELLPVSQASWACNLILGAPDSQKLTTV |
| | | | DIVTLRVLCREGVRWRVMAIQDFKPFENLRL |
| | | | MAPISLQVVHVETHRANISWEISQASHYFER |
| | | | HLEFEARTLSPGHTWEEAPLLTLKQKQEWIT |
| | | | LETLTPDTQYEFQVRVKPLQGEFTTWSPWSQ |
| | | | PLAFRTKPAALGKDT |
| Construct | SEQ ID | M005 | DKTHTCPPCPAPELLGGPSVFLFPPKPKDTLM |
| F2 | NO: 89 | pairs | ISRTPEVTCVVVDVSHEDPEVKFNWYVDGV |
| | | with | EVHNAKTKPREEQYNSTYRVVSVLTVLHQD |
| | | MK5 | WLNGKEYKCKVSNKALPAPIEKTISKAKGQP |
| | | (SEQ ID | REPQVCTLPPSRDELTKNQVSLSCAVKGFYPS |
| | | NO: 90) | DIAVEWESNGQPENNYKTTPPVLDSDGSFFL |
| | | | VSKLTVDKSRWQQGNVFSCSVMHEALHNH |
| | | | YTQKSLSLSPGGGGGSAVNGTSQFTCFYNSR |
| | | | ANISCVWSQDGALQDTSCQVHAWPDRRRW |
| | | | NQTCELLPVSQASWACNLILGAPDSQKLTTV |
| | | | DIVTLRVLCREGVRWRVMAIQDFKPEENLRL |
| | | | MAPISLQVVHVETHRCNISWEISQASHYFER |
| | | | HLEFEARTLSPGHTWEEAPLLTLKQKQEWIC |
| | | | LETLTPDTQYEFQVRVKPLQ |
| | SEQ ID | MK5 | DKTHTCPPCPAPELLGGPSVFLFPPKPKDTLM |
| | NO: 90 | | ISRTPEVTCVVVDVSHEDPEVKFNWYVDGV |
| | | | EVHNAKTKPREEQYNSTYRVVSVLTVLHQD |
| | | | WLNGKEYKCKVSNKALPAPIEKTISKAKGQP |
| | | | REPQVYTLPPCRDELTKNQVSLWCLVKGFYP |
| | | | SDIAVEWESNGQPENNYKTTPPVLDSDGSFF |
| | | | LYSKLTVDKSRWQQGNVFSCSVMHEALHNH |
| | | | YTQKSLSLSPGGGGSSGGSGGSGGIPVSLRSG |
| | | | GGGSNWVNVISDLKKIEDLIQSMHIDATLYTE |
| | | | SDVHPSCKVTAMKCFLLELQVISLESGDASIH |
| | | | DTVENLIILANNSLSSNGNVTESGCKECEELE |
| | | | EKNIKEFLQSFVHIVQMFINTSSGGSGGGGSG |
| | | | GGSGGGGSLQITCPPPMSVEHADIWVKSYSL |
| | | | YSRERYICNSGFKRKAGTSSLTECVLNKATN |
| | | | VAHWTTPSLKCIRDPALVHQRPAPP |
| | SEQ ID | M2006 | DKTHTCPPCPAPELLGGPSVFLFPPKPKDTLM |
| | NO: 91 | pairs | ISRTPEVTCVVVDVSHEDPEVKFNWYVDGV |
| | | with | EVHNAKTKPREEQYASTYRVVSVLTVLHQD |
| | | MK5 | WLNGKEYKCKVSNKALPAPIEKTISKAKGQP |
| | | (SEQ ID | REPQVCTLPPSRDELTKNQVSLSCAVKGFYPS |
| | | NO: 90 | DIAVEWESNGQPENNYKTTPPVLDSDGSFFL |
| | | | VSKLTVDKSRWQQGNVFSCSVMHEALHNH |
| | | | YTQKSLSLSPGGGGGSAVNGTSQFTCFYNSR |
| | | | ANISCVWSQDGALQDTSCQVHAWPDRRRW |
| | | | NQTCELLPVSQASWACNLILGAPDSQKLTTV |
| | | | DIVTLRVLCREGVRWRVMAIQDFKPFENLRL |
| | | | MAPISLQVVHVETHRCNISWEISQASHYFER |
| | | | HLEFEARTLSPGHTWEEAPLLTLKQKQEWIC |
| | | | LETLTPDTQYEFQVRVKPLQGEFTTWSPWSQ |
| | | | PLAFRTKPAALGKD |
| Construct | SEQ ID | M006 | AVNGTSQFTCFYNSRANISCVWSQDGALQDT |
| F3 | NO: 92 | pairs | SCQVHAWPDRRRWNQTCELLPVSQASWACN |
| | | with | LILGAPDSQKLTTVDIVTLRVLCREGVRWRV |
| | | MK6 | MAIQDFKPEENLRLMAPISLQVVHVETHRCN |
| | | (SEQ ID | ISWEISQASHYFERHLEFEARTLSPGHTWEEA |
| | | NO: 93) | PLLTLKQKQEWICLETLTPDTQYEFQVRVKPL |
| | | | QGGGGSDKTHTCPPCPAPELLGGPSVFLFPPK |
| | | | PKDTLMISRTPEVTCVVVDVSHEDPEVKFNW |
| | | | YVDGVEVHNAKTKPREEQYNSTYRVVSVLT |
| | | | VLHQDWLNGKEYKCKVSNKALPAPIEKTISK |
| | | | AKGQPREPQVCTLPPSRDELTKNQVSLSCAV |
| | | | KGFYPSDIAVEWESNGQPENNYKTTPPVLDS |
| | | | DGSFFLVSKLTVDKSRWQQGNVFSCSVMHE |
| | | | ALHNHYTQKSLSLSPG |
| | SEQ ID | MK6 | ITCPPPMSVEHADIWVKSYSLYSRERYICNSG |
| | NO: 93 | | FKRKAGTSSLTECVLNKATNVAHWTTPSLKC |
| | | | IRDPALVHQRPAPPSGGSGGGGSGGGSGGGG |
| | | | SLQNWVNVISDLKKIEDLIQSMHIDATLYTES |
| | | | DVHPSCKVTAMKCFLLELQVISLESGDASIH |
| | | | DTVENLIILANNSLSSNGNVTESGCKECEELE |
| | | | EKNIKEFLQSFVHIVQMFINTSGGGSIPVSLRS |
| | | | GGGGSSGGSGGSGGDKTHTCPPCPAPELLGG |
| | | | PSVFLFPPKPKDTLMISRTPEVTCVVVDVSHE |
| | | | DPEVKFNWYVDGVEVHNAKTKPREEQYNS |
| | | | TYRVVSVLTVLHQDWLNGKEYKCKVSNKA |
| | | | LPAPIEKTISKAKGQPREPQVYTLPPCRDELT |
| | | | KNQVSLWCLVKGFYPSDIAVEWESNGQPEN |
| | | | NYKTTPPVLDSDGSFFLYSKLTVDKSRWQQG |
| | | | NVFSCSVMHEALHNHYTQKSLSLSPG |
| | SEQ ID | M2007 | AVNGTSQFTCFYNSRANISCVWSQDGALQDT |
| | NO: 94 | pairs | SCQVHAWPDRRRWNQTCELLPVSQASWACN |
| | | with | LILGAPDSQKLTTVDIVTLRVLCREGVRWRV |
| | | MK6 | MAIQDFKPFENLRLMAPISLQVVHVETHRCN |
| | | (SEQ ID | ISWEISQASHYFERHLEFEARTLSPGHTWEEA |
| | | NO: 93) | PLLTLKQKQEWICLETLTPDTQYEFQVRVKPL |
| | | | QGEFTTWSPWSQPLAFRTKPAALGKDGGGG |
| | | | SDKTHTCPPCPAPELLGGPSVFLFPPKPKDTL |
| | | | MISRTPEVTCVVVDVSHEDPEVKFNWYVDG |
| | | | VEVHNAKTKPREEQYASTYRVVSVLTVLHQ |
| | | | DWLNGKEYKCKVSNKALPAPIEKTISKAKGQ |
| | | | PREPQVCTLPPSRDELTKNQVSLSCAVKGFYP |
| | | | SDIAVEWESNGQPENNYKTTPPVLDSDGSFF |
| | | | LVSKLTVDKSRWQQGNVFSCSVMHEALHNH |
| | | | YTQKSLSLSPG |
| Construct | SEQ ID | M108 | AVNGTSQFTCFYNSRANISCVWSQDGALQDT |
| G1 | NO: 95 | pairs | SCQVHAWPDRRRWNQTCELLPVSQASWACN |
| | | with | LILGAPDSQKLTTVDIVTLRVLCREGVRWRV |
| | | MH4 | MAIQDFKPFENLRLMAPISLQVVHVETHRCN |
| | | (SEQ ID | ISWEISQASHYFERHLEFEARTLSPGHTWEEA |
| | | NO: 96) | PLLTLKQKQEWICLETLTPDTQYEFQVRVKPL |
| | | | QGEFTTWSPWSQPLAFRTKPAALGKDGGGSI |
| | | | PVSLRSGGGGSSGGSGGSGGNWVNVISDLK |
| | | | KIEDLIQSMHIDATLYTESDVHPSCKVTAMKC |
| | | | FLLELQVISLESGDASIHDTVENLIILANNSLS |
| | | | SNGNVTESGCKECEELEEKNIKEFLQSFVHIV |
| | | | QMFINTSEPKSSDKTHTCPPCPAPELLGGPSV |
| | | | FLFPPKPKDTLMISRTPEVTCVVVDVSHEDPE |
| | | | VKFNWYVDGVEVHNAKTKPREEQYNSTYR |
| | | | VVSVLTVLHQDWLNGKEYKCKVSNKALPAP |
| | | | IEKTISKAKGQPREPQVYTLPPCRDELTKNQV |
| | | | SLWCLVKGFYPSDIAVEWESNGQPENNYKTT |
| | | | PPVLDSDGSFFLYSKLTVDKSRWQQGNVFSC |
| | | | SVMHEALHNHYTQKSLSLSPGK |
| | SEQ ID | MH4 | ITCPPPMSVEHADIWVKSYSLYSRERYICNSG |
| | NO: 96 | | FKRKAGTSSLTECVLNKATNVAHWTTPSLKC |
| | | | IREPKSSDKTHTCPPCPAPELLGGPSVFLFPPK |
| | | | PKDTLMISRTPEVTCVVVDVSHEDPEVKFNW |
| | | | YVDGVEVHNAKTKPREEQYNSTYRVVSVLT |
| | | | VLHQDWLNGKEYKCKVSNKALPAPIEKTISK |
| | | | AKGQPREPQVCTLPPSRDELTKNQVSLSCAV |
| | | | KGFYPSDIAVEWESNGQPENNYKTTPPVLDS |
| | | | DGSFFLVSKLTVDKSRWQQGNVFSCSVMHE |
| | | | ALHNHYTQKSLSLSPGK |
| Construct | SEQ ID | M112 | DKTHTCPPCPAPELLGGPSVFLFPPKPKDTLM |
| G2 | NO: 97 | pairs | ISRTPEVTCVVVDVSHEDPEVKFNWYVDGV |
| | | with | EVHNAKTKPREEQYNSTYRVVSVLTVLHQD |
| | | MK113 | WLNGKEYKCKVSNKALPAPIEKTISKAKGQP |
| | | (SEQ ID | REPQVYTLPPCRDELTKNQVSLWCLVKGFYP |
| | | NO: 98) | SDIAVEWESNGQPENNYKTTPPVLDSDGSFF |
| | | | LYSKLTVDKSRWQQGNVFSCSVMHEALHNH |
| | | | YTQKSLSLSPGGGGGSNWVNVISDLKKIEDL |
| | | | IQSMHIDATLYTESDVHPSCKVTAMKCFLLEL |
| | | | QVISLESGDASIHDTVENLIILANNSLSSNGN |
| | | | VTESGCKECEELEEKNIKEFLQSFVHIVQMFI |
| | | | NTSGGGSIPVSLRSGGGGSSGGSGGSGGAVN |
| | | | GTSQFTCFYNSRANISCVWSQDGALQDTSCQ |
| | | | VHAWPDRRRWNQTCELLPVSQASWACNLIL |
| | | | GAPDSQKLTTVDIVTLRVLCREGVRWRVMAI |
| | | | QDFKPFENLRLMAPISLQVVHVETHRCNISW |
| | | | EISQASHYFERHLEFEARTLSPGHTWEEAPLL |
| | | | TLKQKQEWICLETLTPDTQYEFQVRVKPLQG |
| | | | EFTTWSPWSQPLAFRTKPAALGKD |
| | SEQ ID | MK113 | DKTHTCPPCPAPELLGGPSVFLFPPKPKDTLM |
| | NO: 98 | | ISRTPEVTCVVVDVSHEDPEVKFNWYVDGV |
| | | | EVHNAKTKPREEQYNSTYRVVSVLTVLHQD |
| | | | WLNGKEYKCKVSNKALPAPIEKTISKAKGQP |
| | | | REPQVCTLPPSRDELTKNQVSLSCAVKGFYPS |
| | | | DIAVEWESNGQPENNYKTTPPVLDSDGSFFL |
| | | | VSKLTVDKSRWQQGNVFSCSVMHEALHNH |
| | | | YTQKSLSLSPGGGGGSITCPPPMSVEHADIW |
| | | | VKSYSLYSRERYICNSGFKRKAGTSSLTECVL |
| | | | NKATNVAHWTTPSLKCIR |
| Construct | SEQ ID | M001, | EVQLVESGGGLVQPGGSLRLSCAVSGFSLTSY |
| H1 | NO: 99 | pairs | GVHWVRQAPGKGLEWVAVIWAGGSTNYAD |
| | | with | SVKGRFTISKDTSKNTVYLQMNSLRAEDTAV |
| | | MH333 | YYCAKPYGTSAMDYWGQGTLVTVSSASTK |
| | | LC | GPSVFPLAPSSKSTSGGTAALGCLVKDYFPEP |
| | | (SEQ ID | VTVSWNSGALTSGVHTFPAVLQSSGLYSLSSV |
| | | NO: | VTVPSSSLGTQTYICNVNHKPSNTKVDKKVE |
| | | 100) | PKSCDKTHTCPPCPAPPAAGPSVFLFPPKPKD |
| | | | TLMISRTPEVTCVVVDVSHEDPEVKFNWYV |
| | | | DGVEVHNAKTKPREEQYNSTYRVVSVLTVL |
| | | | HQDWLNGKEYKCKVSNKALAAPIEKTISKA |
| | | | KGQPREPQVYTLPPSRDELTKNQVSLTCLVK |
| | | | GFYPSDIAVEWESNGQPENNYKTTPPVLDSD |
| | | | GSFFLYSKLTVDKSRWQQGNVFSCSVMHEA |
| | | | LHNHYTQKSLSLSPGKGGGGSAVNGTSQFTC |
| | | | FYNSRANISCVWSQDGALQDTSCQVHAWPD |
| | | | RRRWNQTCELLPVSQASWACNLILGAPDSQ |
| | | | KLTTVDIVTLRVLCREGVRWRVMAIQDFKPF |
| | | | ENLRLMAPISLQVVHVETHRCNISWEISQAS |
| | | | HYFERHLEFEARTLSPGHTWEEAPLLTLKQK |
| | | | QEWICLETLTPDTQYEFQVRVKPLQGEFTTW |
| | | | SPWSQPLAFRTKPAALGKDGGGSSGGSGGSG |
| | | | GIPVSLRSGGGGSITCPPPMSVEHADIWVKSY |
| | | | SLYSRERYICNSGFKRKAGTSSLTECVLNKAT |
| | | | NVAHWTTPSLKCIRDPALVHQRPAPPSGGSG |
| | | | GGGSGGGSGGGGSLQNWVNVISDLKKIEDLI |
| | | | QSMHIDATLYTESDVHPSCKVTAMKCFLLEL |
| | | | QVISLESGDASIHDTVENLIILANNSLSSNGN |
| | | | VTESGCKECEELEEKNIKEFLQSFVHIVQMFI |
| | | | NTS |
| | SEQ ID | MH333 | DIQMTQSPSSLSASVGDRVTITCKASQDVGIV |
| | NO: 100 | LC | VAWYQQKPGKAPKLLIYWASIRHTGVPSRFS |
| | | | GSGSGTEFTLTISSLQPDDFATYYCQQYSNYP |
| | | | LYTFGQGTKVEIKRTVAAPSVFIFPPSDEQLKS |
| | | | GTASVVCLLNNFYPREAKVQWKVDNALQSG |
| | | | NSQESVTEQDSKDSTYSLSSTLTLSKADYEK |
| | | | HKVYACEVTHQGLSSPVTKSFNRGEC |
| Construct | SEQ ID | M002 | EVQLVESGGGLVQPGGSLRLSCAVSGFSLTSY |
| H2 | NO: 101 | pairs | GVHWVRQAPGKGLEWVAVIWAGGSTNYAD |
| | | with | SVKGRFTISKDTSKNTVYLQMNSLRAEDTAV |
| | | MH2 | YYCAKPYGTSAMDYWGQGTLVTVSSASTK |
| | | SEQ ID | GPSVFPLAPSSKSTSGGTAALGCLVKDYFPEP |
| | | NO: 102 | VTVSWNSGALTSGVHTFPAVLQSSGLYSLSSV |
| | | and | VTVPSSSLGTQTYICNVNHKPSNTKVDKKVE |
| | | MH333 | PKSCDKTHTCPPCPAPPAAGPSVFLFPPKPKD |
| | | LC SEQ | TLMISRTPEVTCVVVDVSHEDPEVKFNWYV |
| | | ID | DGVEVHNAKTKPREEQYNSTYRVVSVLTVL |
| | | NO: 100 | HQDWLNGKEYKCKVSNKALAAPIEKTISKA |
| | | | KGQPREPQVYTLPPCRDELTKNQVSLWCLVK |
| | | | GFYPSDIAVEWESNGQPENNYKTTPPVLDSD |
| | | | GSFFLYSKLTVDKSRWQQGNVFSCSVMHEA |
| | | | LHNHYTQKSLSLSPGKGGGGSAVNGTSQFTC |
| | | | FYNSRANISCVWSQDGALQDTSCQVHAWPD |
| | | | RRRWNQTCELLPVSQASWACNLILGAPDSQ |
| | | | KLTTVDIVTLRVLCREGVRWRVMAIQDFKPF |
| | | | ENLRLMAPISLQVVHVETHRCNISWEISQAS |
| | | | HYFERHLEFEARTLSPGHTWEEAPLLTLKQK |
| | | | QEWICLETLTPDTQYEFQVRVKPLQGEFTTW |
| | | | SPWSQPLAFRTKPAALGKDGGGSSGGSGGSG |
| | | | GIPVSLRSGGGGSITCPPPMSVEHADIWVKSY |
| | | | SLYSRERYICNSGFKRKAGTSSLTECVLNKAT |
| | | | NVAHWTTPSLKCIRDPALVHQRPAPPSGGSG |
| | | | GGGSGGGSGGGGSLQNWVNVISDLKKIEDLI |
| | | | QSMHIDATLYTESDVHPSCKVTAMKCFLLEL |
| | | | QVISLESGDASIHDTVENLIILANNSLSSNGN |
| | | | VTESGCKECEELEEKNIKEFLQSFVHIVQMFI |
| | | | NTS |
| | SEQ ID | MH2 | EVQLVESGGGLVQPGGSLRLSCAVSGFSLTSY |
| | NO: 102 | | GVHWVRQAPGKGLEWVAVIWAGGSTNYAD |
| | | | SVKGRFTISKDTSKNTVYLQMNSLRAEDTAV |
| | | | YYCAKPYGTSAMDYWGQGTLVTVSSASTK |
| | | | GPSVFPLAPSSKSTSGGTAALGCLVKDYFPEP |
| | | | VTVSWNSGALTSGVHTFPAVLQSSGLYSLSSV |
| | | | VTVPSSSLGTQTYICNVNHKPSNTKVDKKVE |
| | | | PKSCDKTHTCPPCPAPPAAGPSVFLFPPKPKD |
| | | | TLMISRTPEVTCVVVDVSHEDPEVKFNWYV |
| | | | DGVEVHNAKTKPREEQYNSTYRVVSVLTVL |
| | | | HQDWLNGKEYKCKVSNKALAAPIEKTISKA |
| | | | KGQPREPQVCTLPPSRDELTKNQVSLSCAVK |
| | | | GFYPSDIAVEWESNGQPENNYKTTPPVLDSD |
| | | | GSFFLVSKLTVDKSRWQQGNVFSCSVMHEA |
| | | | LHNHYTQKSLSLSPGK |
| | SEQ ID | MH333 | DIQMTQSPSSLSASVGDRVTITCKASQDVGIV |
| | NO: 100 | LC | VAWYQQKPGKAPKLLIYWASIRHTGVPSRFS |
| | | | GSGSGTEFTLTISSLQPDDFATYYCQQYSNYP |
| | | | LYTFGQGTKVEIKRTVAAPSVFIFPPSDEQLKS |
| | | | GTASVVCLLNNFYPREAKVQWKVDNALQSG |
| | | | NSQESVTEQDSKDSTYSLSSTLTLSKADYEK |
| | | | HKVYACEVTHQGLSSPVTKSFNRGEC |
| Construct | SEQ ID | MK3 | EVQLVESGGGLVQPGGSLRLSCAVSGFSLTSY |
| K1 | NO: 103 | pairs | GVHWVRQAPGKGLEWVAVIWAGGSTNYAD |
| | | with | SVKGRFTISKDTSKNTVYLQMNSLRAEDTAV |
| | | MH3 | YYCAKPYGTSAMDYWGQGTLVTVSSASTK |
| | | (SEQ ID | GPSVFPLAPSSKSTSGGTAALGCLVKDYFPEP |
| | | NO: 104 | VTVSWNSGALTSGVHTFPAVLQSSGLYSLSSV |
| | | and | VTVPSSSLGTQTYICNVNHKPSNTKVDKKVE |
| | | MH333 | PKSCDKTHTCPPCPAPPAAGPSVFLFPPKPKD |
| | | LC | TLMISRTPEVTCVVVDVSHEDPEVKFNWYV |
| | | (SEQ ID | DGVEVHNAKTKPREEQYNSTYRVVSVLTVL |
| | | NO: 100) | HQDWLNGKEYKCKVSNKALAAPIEKTISKA |
| | | | KGQPREPQVYTLPPCRDELTKNQVSLWCLVK |
| | | | GFYPSDIAVEWESNGQPENNYKTTPPVLDSD |
| | | | GSFFLYSKLTVDKSRWQQGNVFSCSVMHEA |
| | | | LHNHYTQKSLSLSPGKGGGSIPVSLRSGGGG |
| | | | SITCPPPMSVEHADIWVKSYSLYSRERYICNS |
| | | | GFKRKAGTSSLTECVLNKATNVAHWTTPSLK |
| | | | CIRDPALVHQRPAPPSGGSGGGGSGGGSGGG |
| | | | GSLQNWVNVISDLKKIEDLIQSMHIDATLYTE |
| | | | SDVHPSCKVTAMKCFLLELQVISLESGDASIH |
| | | | DTVENLIILANNSLSSNGNVTESGCKECEELE |
| | | | EKNIKEFLQSFVHIVQMFINTS |
| | SEQ ID | MH3 | EVQLVESGGGLVQPGGSLRLSCAVSGFSLTSY |
| | NO: 104 | | GVHWVRQAPGKGLEWVAVIWAGGSTNYAD |
| | | | SVKGRFTISKDTSKNTVYLQMNSLRAEDTAV |
| | | | YYCAKPYGTSAMDYWGQGTLVTVSSASTK |
| | | | GPSVFPLAPSSKSTSGGTAALGCLVKDYFPEP |
| | | | VTVSWNSGALTSGVHTFPAVLQSSGLYSLSSV |
| | | | VTVPSSSLGTQTYICNVNHKPSNTKVDKKVE |
| | | | PKSCDKTHTCPPCPAPPAAGPSVFLFPPKPKD |
| | | | TLMISRTPEVTCVVVDVSHEDPEVKFNWYV |
| | | | DGVEVHNAKTKPREEQYNSTYRVVSVLTVL |
| | | | HQDWLNGKEYKCKVSNKALAAPIEKTISKA |
| | | | KGQPREPQVCTLPPSRDELTKNQVSLSCAVK |
| | | | GFYPSDIAVEWESNGQPENNYKTTPPVLDSD |
| | | | GSFFLVSKLTVDKSRWQQGNVFSCSVMHEA |
| | | | LHNHYTQKSLSLSPGKGGGGSAVNGTSQFTC |
| | | | FYNSRANISCVWSQDGALQDTSCQVHAWPD |
| | | | RRRWNQTCELLPVSQASWACNLILGAPDSQ |
| | | | KLTTVDIVTLRVLCREGVRWRVMAIQDFKPF |
| | | | ENLRLMAPISLQVVHVETHRANISWEISQAS |
| | | | HYFERHLEFEARTLSPGHTWEEAPLLTLKQK |
| | | | QEWITLETLTPDTQYEFQVRVKPLQGEFTTW |
| | | | SPWSQPLAFRTKPAALGKDT |
| | SEQ ID | MH333 | DIQMTQSPSSLSASVGDRVTITCKASQDVGIV |
| | NO: 100 | LC | VAWYQQKPGKAPKLLIYWASIRHTGVPSRFS |
| | | | GSGSGTEFTLTISSLQPDDFATYYCQQYSNYP |
| | | | LYTFGQGTKVEIKRTVAAPSVFIFPPSDEQLKS |
| | | | GTASVVCLLNNFYPREAKVQWKVDNALQSG |
| | | | NSQESVTEQDSKDSTYSLSSTLTLSKADYEK |
| | | | HKVYACEVTHQGLSSPVTKSFNRGEC |
| Construct | SEQ ID | MK4 | EVQLVESGGGLVQPGGSLRLSCAVSGFSLTSY |
| K2 | NO: 105 | pairs | GVHWVRQAPGKGLEWVAVIWAGGSTNYAD |
| | | with | SVKGRFTISKDTSKNTVYLQMNSLRAEDTAV |
| | | MH3 | YYCAKPYGTSAMDYWGQGTLVTVSSASTK |
| | | (SEQ ID | GPSVFPLAPSSKSTSGGTAALGCLVKDYFPEP |
| | | NO: 106) | VTVSWNSGALTSGVHTFPAVLQSSGLYSLSSV |
| | | and | VTVPSSSLGTQTYICNVNHKPSNTKVDKKVE |
| | | MH333 | PKSCDKTHTCPPCPAPPAAGPSVFLFPPKPKD |
| | | LC | TLMISRTPEVTCVVVDVSHEDPEVKFNWYV |
| | | (SEQ ID | DGVEVHNAKTKPREEQYNSTYRVVSVLTVL |
| | | NO: 100) | HQDWLNGKEYKCKVSNKALAAPIEKTISKA |
| | | | KGQPREPQVYTLPPCRDELTKNQVSLWCLVK |
| | | | GFYPSDIAVEWESNGQPENNYKTTPPVLDSD |
| | | | GSFFLYSKLTVDKSRWQQGNVFSCSVMHEA |
| | | | LHNHYTQKSLSLSPGKGGGSSGGSGGSGGIP |
| | | | VSLRSGGGGSNWVNVISDLKKIEDLIQSMHI |
| | | | DATLYTESDVHPSCKVTAMKCFLLELQVISLE |
| | | | SGDASIHDTVENLIILANNSLSSNGNVTESGC |
| | | | KECEELEEKNIKEFLQSFVHIVQMFINTSSGG |
| | | | SGGGGSGGGSGGGGSLQITCPPPMSVEHADI |
| | | | WVKSYSLYSRERYICNSGFKRKAGTSSLTEC |
| | | | VLNKATNVAHWTTPSLKCIRDPALVHQRPAP |
| | | | P |
| | SEQ ID | MH3 | EVQLVESGGGLVQPGGSLRLSCAVSGFSLTSY |
| | NO: 106 | | GVHWVRQAPGKGLEWVAVIWAGGSTNYAD |
| | | | SVKGRFTISKDTSKNTVYLQMNSLRAEDTAV |
| | | | YYCAKPYGTSAMDYWGQGTLVTVSSASTK |
| | | | GPSVFPLAPSSKSTSGGTAALGCLVKDYFPEP |
| | | | VTVSWNSGALTSGVHTFPAVLQSSGLYSLSSV |
| | | | VTVPSSSLGTQTYICNVNHKPSNTKVDKKVE |
| | | | PKSCDKTHTCPPCPAPPAAGPSVFLFPPKPKD |
| | | | TLMISRTPEVTCVVVDVSHEDPEVKFNWYV |
| | | | DGVEVHNAKTKPREEQYNSTYRVVSVLTVL |
| | | | HQDWLNGKEYKCKVSNKALAAPIEKTISKA |
| | | | KGQPREPQVCTLPPSRDELTKNQVSLSCAVK |
| | | | GFYPSDIAVEWESNGQPENNYKTTPPVLDSD |
| | | | GSFFLVSKLTVDKSRWQQGNVFSCSVMHEA |
| | | | LHNHYTQKSLSLSPGKGGGGSAVNGTSQFTC |
| | | | FYNSRANISCVWSQDGALQDTSCQVHAWPD |
| | | | RRRWNQTCELLPVSQASWACNLILGAPDSQ |
| | | | KLTTVDIVTLRVLCREGVRWRVMAIQDFKPF |
| | | | ENLRLMAPISLQVVHVETHRANISWEISQAS |
| | | | HYFERHLEFEARTLSPGHTWEEAPLLTLKQK |
| | | | QEWITLETLTPDTQYEFQVRVKPLQGEFTTW |
| | | | SPWSQPLAFRTKPAALGKDT |
| | SEQ ID | MH333 | DIQMTQSPSSLSASVGDRVTITCKASQDVGIV |
| | NO: 100 | LC | VAWYQQKPGKAPKLLIYWASIRHTGVPSRFS |
| | | | GSGSGTEFTLTISSLQPDDFATYYCQQYSNYP |
| | | | LYTFGQGTKVEIKRTVAAPSVFIFPPSDEQLKS |
| | | | GTASVVCLLNNFYPREAKVQWKVDNALQSG |
| | | | NSQESVTEQDSKDSTYSLSSTLTLSKADYEK |
| | | | HKVYACEVTHQGLSSPVTKSFNRGEC |
| Construct M | SEQ ID | MK143 | GTSQFTCFYNSRAQISCVWSQDGALQDTSCQ |
| | NO: 107 | Pair | VHAWPDRRRWNQTCELLPVSQASWACNLIL |
| | | with | GAPDSQKLTTVDIVTLRVLCREGVRWRVMAI |
| | | MK144 | QDFKPFENLRLMAPISLQVVHVETHRAQISW |
| | | (SEQ ID | EISQASHYFERHLEFEARTLSPGHTWEEAPLL |
| | | NO: 108) | TLKQKQEWISLETLTPDTQYEFQVRVKPLQG |
| | | and | EFTTWSPWSQPLAFRTKPAGGGSVPLSLYSG |
| | | MH7 | RSASGGSGGGGSGSGNWVNVISDLKKIEDLI |
| | | (SEQ ID | QSMHIDATLYTESDVHPSCKVTAMKCFLLEL |
| | | NO: 52) | QVISCESGDASIHDTVENLIILAQDSLSSNGN |
| | | | VTESGCKECEELEEKNIKEFLQSFVHIVQMFI |
| | | | QTS |
| | SEQ ID | MK144 | ITCPPPMSVEHADIWVKSYSLYSRERYICNSG |
| | NO: 108 | | FKRKAGTCSLTECVLNKATNVAHWTTPSLKC |
| | | | IRDPALVHQREPKSSDKTHTCPPCPAPELLGG |
| | | | PSVFLFPPKPKDTLMISRTPEVTCVVVDVSHE |
| | | | DPEVKFNWYVDGVEVHNAKTKPREEQYNS |
| | | | TYRVVSVLTVLHQDWLNGKEYKCKVSNKA |
| | | | LPAPIEKTISKAKGQPREPQVYTLPPCRDELT |
| | | | KNQVSLWCLVKGFYPSDIAVEWESNGQPEN |
| | | | NYKTTPPVLDSDGSFFLYSKLTVDKSRWQQG |
| | | | NVFSCSVMHEALHNHYTQKSLSLSPGK |
| Construct N | SEQ ID | MK142 | DKTHTCPPCPAPELLGGPSVFLFPPKPKDTLM |
| | NO: 109 | Pair | ISRTPEVTCVVVDVSHEDPEVKFNWYVDGV |
| | | with | EVHNAKTKPREEQYNSTYRVVSVLTVLHQD |
| | | MH7 | WLNGKEYKCKVSNKALPAPIEKTISKAKGQP |
| | | (SEQ ID | REPQVYTLPPCRDELTKNQVSLWCLVKGFYP |
| | | NO: 52) | SDIAVEWESNGQPENNYKTTPPVLDSDGSFF |
| | | | LYSKLTVDKSRWQQGNVFSCSVMHEALHNH |
| | | | YTQKSLSLSPGKGGGGSGGGGSITCPPPMSV |
| | | | EHADIWVKSYSLYSRERYICNSGFKRKAGTS |
| | | | SLTECVLNKATNVAHWTTPSLKCIRDPALVH |
| | | | QRPAPPSGGSGGGGSGGGSGGGGSLQNWVN |
| | | | VISDLKKIEDLIQSMHIDATLYTESDVHPSCK |
| | | | VTAMKCFLLELQVISLESGDASIHDTVENLIIL |
| | | | AQNSLSSNGNVTESGCKECEELEEKNIKEFL |
| | | | QSFVHIVQMFIQTSGGGSVPLSLYSGRSASGG |
| | | | SGGGGSGSGGTSQFTCFYNSRAQISCVWSQD |
| | | | GALQDTSCQVHAWPDRRRWNQTCELLPVSQ |
| | | | ASWACNLILGAPDSQKLTTVDIVTLRVLCRE |
| | | | GVRWRVMAIQDFKPFENLRLMAPISLQVVH |
| | | | VETHRAQISWEISQASHYFERHLEFEARTLSP |
| | | | GHTWEEAPLLTLKQKQEWISLETLTPDTQYE |
| | | | FQVRVKPLQGEFTTWSPWSQPLAFRTKPA |
| | SEQ ID | MK156 | DKTHTCPPCPAPELLGGPSVFLFPPKPKDTLM |
| | NO: 173 | pairs | ISRTPEVTCVVVDVSHEDPEVKFNWYVDGV |
| | | with | EVHNAKTKPREEQYNSTYRVVSVLTVLHQD |
| | | MH8 | WLNGKEYKCKVSNKALPAPIEKTISKAKGQP |
| | | (SEQ ID | REPQVYTLPPSRDELTKNQVSLWCLVKGFYP |
| | | NO: 75) | SDIAVEWESNGQPENNYKTTPPVLDSDGSFF |
| | | | LYSKLTVDKSRWQQGNVFSCSVMHEALHNH |
| | | | YTQKSLSLSPGKGGGGSGGGGSITCPPPMSV |
| | | | EHADIWVKSYSLYSRERYICNSGFKRKAGTS |
| | | | SLTECVLNKATNVAHWTTPSLKCIRDPALVH |
| | | | QRPAPPSGGSGGGGSGGGSGGGGSLQNWVN |
| | | | VISDLKKIEDLIQSMHIDATLYTESDVHPSCK |
| | | | VTAMKCFLLELQVISLESGDASIHDTVENLIIL |
| | | | AQNSLSSNGQVTESGCKECEELEEKNIKEFL |
| | | | QSFVHIVQMFIQTSGGGSVPLSLYSGRSASGG |
| | | | SGGGGSGTSQFTCFYNSRAQISCVWSQDGAL |
| | | | QDTSCQVHAWPDRRRWNQTCELLPVSQAS |
| | | | WACNLILGAPDSQKLTTVDIVTLRVLCREGV |
| | | | RWRVMAIQDFKPFENLRLMAPISLQVVHVET |
| | | | HRAQISWEISQASHYFERHLEFEARTLSPGHT |
| | | | WEEAPLLTLKQKQEWISLETLTPDTQYEFQV |
| | | | RVKPLQGEFTTWSPWSQPLAFRTKPA |
| | SEQ ID | MK165 | DKTHTCPPCPAPELLGGPSVFLFPPKPKDTLM |
| | NO: 174 | pairs | ISRTPEVTCVVVDVSHEDPEVKFNWYVDGV |
| | | with | EVHNAKTKPREEQYNSTYRVVSVLTVLHQD |
| | | MH8 | WLNGKEYKCKVSNKALPAPIEKTISKAKGQP |
| | | (SEQ ID | REPQVYTLPPSRDELTKNQVSLWCLVKGFYP |
| | | NO: 75) | SDIAVEWESNGQPENNYKTTPPVLDSDGSFF |
| | | | LYSKLTVDKSRWQQGNVFSCSVMHEALHNH |
| | | | YTQKSLSLSPGKGGGGSITCPPPMSVEHADI |
| | | | WVKSYSLYSRERYICNSGFKRKAGTSSLTEC |
| | | | VLNKATNVAHWTTPSLKCIRDPALVHQRPAP |
| | | | PSGGSGGGGSGGGSGGGGSLQNWVNVISDL |
| | | | KKIEDLIQSMHIDATLYTESDVHPSCKVTAMK |
| | | | CFLLELQVISLESGDASIHDTVENLIILAQNSL |
| | | | SSNGQVTESGCKECEELEEKNIKEFLQSFVHI |
| | | | VQMFIQTSGGGSVPLSLYSGRSASGGSGGGG |
| | | | SGTSQFTCFYNSRAQISCVWSQDGALQDTSC |
| | | | QVHAWPDRRRWNQTCELLPVSQASWACNLI |
| | | | LGAPDSQKLTTVDIVTLRVLCREGVRWRVM |
| | | | AIQDFKPFENLRLMAPISLQVVHVETHRAQIS |
| | | | WEISQASHYFERHLEFEARTLSPGHTWEEAP |
| | | | LLTLKQKQEWISLETLTPDTQYEFQVRVKPL |
| | | | QGEFTTWSPWSQPLAFRTKPA |
| Construct P | SEQ ID | MK147 | ITCPPPMSVEHADIWVKSYSLYSRERYICNSG |
| | NO: 110 | Pair | FKRKAGTCSLTECVLNKATNVAHWTTPSLKC |
| | | with | IRDPALVHQR |
| | | MK148 | |
| | | (SEQ ID | |
| | | NO: 111) | |
| | | and | |
| | | MH7 | |
| | | (SEQ ID | |
| | | NO: 52) | |
| | SEQ ID | MK148 | NWVNVISDLKKIEDLIQSMHIDATLYTESDVH |
| | NO: 111 | | PSCKVTAMKCFLLELQVISCESGDASIHDTVE |
| | | | NLIILAQDSLSSNGNVTESGCKECEELEEKNI |
| | | | KEFLQSFVHIVQMFIQTSGGGSVPLSLYSGRS |
| | | | ASGGSGGGGSGSGGTSQFTCFYNSRAQISCV |
| | | | WSQDGALQDTSCQVHAWPDRRRWNQTCEL |
| | | | LPVSQASWACNLILGAPDSQKLTTVDIVTLRV |
| | | | LCREGVRWRVMAIQDFKPFENLRLMAPISLQ |
| | | | VVHVETHRAQISWEISQASHYFERHLEFEAR |
| | | | TLSPGHTWEEAPLLTLKQKQEWISLETLTPDT |
| | | | QYEFQVRVKPLQGEFTTWSPWSQPLAFRTKP |
| | | | AGGGGSGGGGSDKTHTCPPCPAPELLGGPSV |
| | | | FLFPPKPKDTLMISRTPEVTCVVVDVSHEDPE |
| | | | VKFNWYVDGVEVHNAKTKPREEQYNSTYR |
| | | | VVSVLTVLHQDWLNGKEYKCKVSNKALPAP |
| | | | IEKTISKAKGQPREPQVYTLPPCRDELTKNQV |
| | | | SLWCLVKGFYPSDIAVEWESNGQPENNYKTT |
| | | | PPVLDSDGSFFLYSKLTVDKSRWQQGNVFSC |
| | | | SVMHEALHNHYTQKSLSLSPGK |
| Construct Q | SEQ ID | MK14 | EVQLVESGGGLVQPGGSLRLSCAVSGFSLTSY |
| | NO: 175 | pairs | GVHWVRQAPGKGLEWVAVIWAGGSTNYAD |
| | | with | SVKGRFTISKDTSKNTVYLQMNSLRAEDTAV |
| | | MH2 | YYCAKPYGTSAMDYWGQGTLVTVSSASTK |
| | | (SEQ ID | GPSVFPLAPSSKSTSGGTAALGCLVKDYFPEP |
| | | NO: | VTVSWNSGALTSGVHTFPAVLQSSGLYSLSSV |
| | | 102) and | VTVPSSSLGTQTYICNVNHKPSNTKVDKKVE |
| | | MH333 | PKSCDKTHTCPPCPAPPAAGPSVFLFPPKPKD |
| | | LC | TLMISRTPEVTCVVVDVSHEDPEVKFNWYV |
| | | (SEQ ID | DGVEVHNAKTKPREEQYNSTYRVVSVLTVL |
| | | NO: | HQDWLNGKEYKCKVSNKALAAPIEKTISKA |
| | | 100) | KGQPREPQVYTLPPCRDELTKNQVSLWCLVK |
| | | | GFYPSDIAVEWESNGQPENNYKTTPPVLDSD |
| | | | GSFFLYSKLTVDKSRWQQGNVFSCSVMHEA |
| | | | LHNHYTQKSLSLSPGKGGGGSITCPPPMSVE |
| | | | HADIWVKSYSLYSRERYICNSGFKRKAGTSS |
| | | | LTECVLNKATNVAHWTTPSLKCIRDPALVHQ |
| | | | RPAPPSGGSGGGGSGGGSGGGGSLQNWVNV |
| | | | ISDLKKIEDLIQSMHIDATLYTESDVHPSCKVT |
| | | | AMKCFLLELQVISLESGDASIHDTVENLIILA |
| | | | QNSLSSNGQVTESGCKECEELEEKNIKEFLQS |
| | | | FVHIVQMFIQTSGGGSVPLSLYSGRSASGGSG |
| | | | GGGSGTSQFTCFYNSRAQISCVWSQDGALQ |
| | | | DTSCQVHAWPDRRRWNQTCELLPVSQASWA |
| | | | CNLILGAPDSQKLTTVDIVTLRVLCREGVRW |
| | | | RVMAIQDFKPFENLRLMAPISLQVVHVETHR |
| | | | AQISWEISQASHYFERHLEFEARTLSPGHTWE |
| | | | EAPLLTLKQKQEWISLETLTPDTQYEFQVRV |
| | | | KPLQGEFTTWSPWSQPLAFRTKPA |
| | SEQ ID | MH2 | EVQLVESGGGLVQPGGSLRLSCAVSGFSLTSY |
| | NO: 102 | | GVHWVRQAPGKGLEWVAVIWAGGSTNYAD |
| | | | SVKGRFTISKDTSKNTVYLQMNSLRAEDTAV |
| | | | YYCAKPYGTSAMDYWGQGTLVTVSSASTK |
| | | | GPSVFPLAPSSKSTSGGTAALGCLVKDYFPEP |
| | | | VTVSWNSGALTSGVHTFPAVLQSSGLYSLSSV |
| | | | VTVPSSSLGTQTYICNVNHKPSNTKVDKKVE |
| | | | PKSCDKTHTCPPCPAPPAAGPSVFLFPPKPKD |
| | | | TLMISRTPEVTCVVVDVSHEDPEVKFNWYV |
| | | | DGVEVHNAKTKPREEQYNSTYRVVSVLTVL |
| | | | HQDWLNGKEYKCKVSNKALAAPIEKTISKA |
| | | | KGQPREPQVCTLPPSRDELTKNQVSLSCAVK |
| | | | GFYPSDIAVEWESNGQPENNYKTTPPVLDSD |
| | | | GSFFLVSKLTVDKSRWQQGNVFSCSVMHEA |
| | | | LHNHYTQKSLSLSPGK |
| | SEQ ID | MH333 | DIQMTQSPSSLSASVGDRVTITCKASQDVGIV |
| | NO: 100 | LC | VAWYQQKPGKAPKLLIYWASIRHTGVPSRFS |
| | | | GSGSGTEFTLTISSLQPDDFATYYCQQYSNYP |
| | | | LYTFGQGTKVEIKRTVAAPSVFIFPPSDEQLKS |
| | | | GTASVVCLLNNFYPREAKVQWKVDNALQSG |
| | | | NSQESVTEQDSKDSTYSLSSTLTLSKADYEK |
| | | | HKVYACEVTHQGLSSPVTKSFNRGEC |
| IgGGHG1- | SEQ ID | IgHG1 | EPKSCDKTHTCPPCPAPELLGGPSVFLFPPKP |
| Fc | NO: 112 | (99-330) | KDTLMISRTPEVTCVVVDVSHEDPEVKFNW |
| | | | YVDGVEVHNAKTKPREEQYNSTYRVVSVLT |
| | | | VLHQDWLNGKEYKCKVSNKALPAPIEKTISK |
| | | | AKGQPREPQVYTLPPSRDELTKNQVSLTCLV |
| | | | KGFYPSDIAVEWESNGQPENNYKTTPPVLDS |
| | | | DGSFFLYSKLTVDKSRWQQGNVFSCSVMHE |
| | | | ALHNHYTQKSLSLSPGK |
| | SEQ ID | IgHG1 | EPKSSDKTHTCPPCPAPELLGGPSVFLFPPKP |
| | NO: 113 | (99-330, | KDTLMISRTPEVTCVVVDVSHEDPEVKFNW |
| | | C103S) | YVDGVEVHNAKTKPREEQYNSTYRVVSVLT |
| | | | VLHQDWLNGKEYKCKVSNKALPAPIEKTISK |
| | | | AKGQPREPQVYTLPPSRDELTKNQVSLTCLV |
| | | | KGFYPSDIAVEWESNGQPENNYKTTPPVLDS |
| | | | DGSFFLYSKLTVDKSRWQQGNVFSCSVMHE |
| | | | ALHNHYTQKSLSLSPGK |
| | SEQ ID | IgHG1 | DKTHTCPPCPAPELLGGPSVFLFPPKPKDTLM |
| | NO: 114 | (104- | ISRTPEVTCVVVDVSHEDPEVKFNWYVDGV |
| | | 330) | EVHNAKTKPREEQYNSTYRVVSVLTVLHQD |
| | | | WLNGKEYKCKVSNKALPAPIEKTISKAKGQP |
| | | | REPQVYTLPPSRDELTKNQVSLTCLVKGFYPS |
| | | | DIAVEWESNGQPENNYKTTPPVLDSDGSFFL |
| | | | YSKLTVDKSRWQQGNVFSCSVMHEALHNH |
| | | | YTQKSLSLSPGK |
| | SEQ ID | IgHG1 | EPKSSDKTHTCPPCPAPELLGGPSVFLFPPKP |
| | NO: 115 | (99-329, | KDTLMISRTPEVTCVVVDVSHEDPEVKFNW |
| | | C103S) | YVDGVEVHNAKTKPREEQYNSTYRVVSVLT |
| | | | VLHQDWLNGKEYKCKVSNKALPAPIEKTISK |
| | | | AKGQPREPQVYTLPPSRDELTKNQVSLTCLV |
| | | | KGFYPSDIAVEWESNGQPENNYKTTPPVLDS |
| | | | DGSFFLYSKLTVDKSRWQQGNVFSCSVMHE |
| | | | ALHNHYTQKSLSLSPG |
| | SEQ ID | IgHG1 | DKTHTCPPCPAPELLGGPSVFLFPPKPKDTLM |
| | NO: 116 | (104- | ISRTPEVTCVVVDVSHEDPEVKFNWYVDGV |
| | | 329) | EVHNAKTKPREEQYNSTYRVVSVLTVLHQD |
| | | | WLNGKEYKCKVSNKALPAPIEKTISKAKGQP |
| | | | REPQVYTLPPSRDELTKNQVSLTCLVKGFYPS |
| | | | DIAVEWESNGQPENNYKTTPPVLDSDGSFFL |
| | | | YSKLTVDKSRWQQGNVFSCSVMHEALHNH |
| | | | YTQKSLSLSPG |
| | SEQ ID | IgHG1 | EPKSSDKTHTCPPCPAPELLGGPSVFLFPPKP |
| | NO: 117 | (99-330, | KDTLMISRTPEVTCVVVDVSHEDPEVKFNW |
| | | C103S, | YVDGVEVHNAKTKPREEQYNSTYRVVSVLT |
| | | S354C, | VLHQDWLNGKEYKCKVSNKALPAPIEKTISK |
| | | T366W) | AKGQPREPQVYTLPPCRDELTKNQVSLWCLV |
| | | knob | KGFYPSDIAVEWESNGQPENNYKTTPPVLDS |
| | | | DGSFFLYSKLTVDKSRWQQGNVFSCSVMHE |
| | | | ALHNHYTQKSLSLSPGK |
| | SEQ ID | IgHG1 | EPKSSDKTHTCPPCPAPELLGGPSVFLFPPKP |
| | NO: 118 | (99-330, | KDTLMISRTPEVTCVVVDVSHEDPEVKFNW |
| | | C103S, | YVDGVEVHNAKTKPREEQYNSTYRVVSVLT |
| | | Y349C, | VLHQDWLNGKEYKCKVSNKALPAPIEKTISK |
| | | T366S, | AKGQPREPQVCTLPPSRDELTKNQVSLSCAV |
| | | L368A, | KGFYPSDIAVEWESNGQPENNYKTTPPVLDS |
| | | Y407V) | DGSFFLVSKLTVDKSRWQQGNVFSCSVMHE |
| | | hole | ALHNHYTQKSLSLSPGK |
| | SEQ ID | IgHG1 | EPKSSDKTHTCPPCPAPELLGGPSVFLFPPKP |
| | NO: 119 | (99-329, | KDTLMISRTPEVTCVVVDVSHEDPEVKFNW |
| | | C103S, | YVDGVEVHNAKTKPREEQYNSTYRVVSVLT |
| | | S354C, | VLHQDWLNGKEYKCKVSNKALPAPIEKTISK |
| | | T366W) | AKGQPREPQVYTLPPCRDELTKNQVSLWCLV |
| | | knob | KGFYPSDIAVEWESNGQPENNYKTTPPVLDS |
| | | | DGSFFLYSKLTVDKSRWQQGNVFSCSVMHE |
| | | | ALHNHYTQKSLSLSPG |
| | SEQ ID | IgHG1 | EPKSSDKTHTCPPCPAPELLGGPSVFLFPPKP |
| | NO: 120 | (99-329, | KDTLMISRTPEVTCVVVDVSHEDPEVKFNW |
| | | C103S, | YVDGVEVHNAKTKPREEQYNSTYRVVSVLT |
| | | Y349C, | VLHQDWLNGKEYKCKVSNKALPAPIEKTISK |
| | | T366S, | AKGQPREPQVCTLPPSRDELTKNQVSLSCAV |
| | | L368A, | KGFYPSDIAVEWESNGQPENNYKTTPPVLDS |
| | | Y407V) | DGSFFLVSKLTVDKSRWQQGNVFSCSVMHE |
| | | hole | ALHNHYTQKSLSLSPG |
| | SEQ ID | IgHG1 | DKTHTCPPCPAPELLGGPSVFLFPPKPKDTLM |
| | NO: 121 | (104- | ISRTPEVTCVVVDVSHEDPEVKFNWYVDGV |
| | | 330, | EVHNAKTKPREEQYNSTYRVVSVLTVLHQD |
| | | S354C, | WLNGKEYKCKVSNKALPAPIEKTISKAKGQP |
| | | T366W) | REPQVYTLPPCRDELTKNQVSLWCLVKGFYP |
| | | knob | SDIAVEWESNGQPENNYKTTPPVLDSDGSFF |
| | | | LYSKLTVDKSRWQQGNVFSCSVMHEALHNH |
| | | | YTQKSLSLSPGK |
| | SEQ ID | IgHG1 | DKTHTCPPCPAPELLGGPSVFLFPPKPKDTLM |
| | NO: 122 | (104- | ISRTPEVTCVVVDVSHEDPEVKFNWYVDGV |
| | | 330, | EVHNAKTKPREEQYNSTYRVVSVLTVLHQD |
| | | Y349C, | WLNGKEYKCKVSNKALPAPIEKTISKAKGQP |
| | | T366S, | REPQVCTLPPSRDELTKNQVSLSCAVKGFYPS |
| | | L368A, | DIAVEWESNGQPENNYKTTPPVLDSDGSFFL |
| | | Y407V) | VSKLTVDKSRWQQGNVFSCSVMHEALHNH |
| | | hole | YTQKSLSLSPGK |
| | SEQ ID | IgHG1 | DKTHTCPPCPAPELLGGPSVFLFPPKPKDTLM |
| | NO: 123 | (104- | ISRTPEVTCVVVDVSHEDPEVKFNWYVDGV |
| | | 329, | EVHNAKTKPREEQYNSTYRVVSVLTVLHQD |
| | | S354C, | WLNGKEYKCKVSNKALPAPIEKTISKAKGQP |
| | | T366W) | REPQVYTLPPCRDELTKNQVSLWCLVKGFYP |
| | | knob | SDIAVEWESNGQPENNYKTTPPVLDSDGSFF |
| | | | LYSKLTVDKSRWQQGNVFSCSVMHEALHNH |
| | | | YTQKSLSLSPG |
| | SEQ ID | IgHG1 | DKTHTCPPCPAPELLGGPSVFLFPPKPKDTLM |
| | NO: 124 | (104- | ISRTPEVTCVVVDVSHEDPEVKFNWYVDGV |
| | | 329, | EVHNAKTKPREEQYNSTYRVVSVLTVLHQD |
| | | Y349C, | WLNGKEYKCKVSNKALPAPIEKTISKAKGQP |
| | | T366S, | REPQVCTLPPSRDELTKNQVSLSCAVKGFYPS |
| | | L368A, | DIAVEWESNGQPENNYKTTPPVLDSDGSFFL |
| | | Y407V) | VSKLTVDKSRWQQGNVFSCSVMHEALHNH |
| | | hole | YTQKSLSLSPG |
| | SEQ ID | IGHG1 | DKTHTCPPCPAPELLGGPSVFLFPPKPKDTLM |
| | NO: 125 | (104- | ISRTPEVTCVVVDVSHEDPEVKFNWYVDGV |
| | | 330, | EVHNAKTKPREEQYNSTYRVVSVLTVLHQD |
| | | T366W), | WLNGKEYKCKVSNKALPAPIEKTISKAKGQP |
| | | knob | REPQVYTLPPSRDELTKNQVSLWCLVKGFYP |
| | | | SDIAVEWESNGQPENNYKTTPPVLDSDGSFF |
| | | | LYSKLTVDKSRWQQGNVFSCSVMHEALHNH |
| | | | YTQKSLSLSPGK |
| | SEQ ID | IGHG1 | DKTHTCPPCPAPELLGGPSVFLFPPKPKDTLM |
| | NO: 126 | (104- | ISRTPEVTCVVVDVSHEDPEVKFNWYVDGV |
| | | 330, | EVHNAKTKPREEQYNSTYRVVSVLTVLHQD |
| | | T366S, | WLNGKEYKCKVSNKALPAPIEKTISKAKGQP |
| | | L368A, | REPQVYTLPPSRDELTKNQVSLSCAVKGFYPS |
| | | Y407V) | DIAVEWESNGQPENNYKTTPPVLDSDGSFFL |
| | | hole | VSKLTVDKSRWQQGNVFSCSVMHEALHNH |
| | | | YTQKSLSLSPGK |

### Linkers

It is also understood that the domains and/or regions of the polypeptide chains of the IL15 constructs can be contain linker regions of various lengths. In some embodiments, the IL15 construct domains are separated from each other by a linker region. For example, IL15Ra-(linker)-IL15. In some aspects, the linker can contain a protease activatable (cleavable) moiety.

In some embodiments, the amino acids glycine and serine comprise the amino acids of the linker (a “GS” linker). In another embodiment, the linker can be, without limitation the linkers in Table 3 or any combination thereof.

TABLE 3

| | | AA |
|---|---|---|
| SEQ ID NO: | SEQUENCE | Length |
| SEQ ID NO: 127 | SGGSGGGGSGGGSGGGGSLQ | 20 |
| SEQ ID NO: 128 | GGS | 3 |
| SEQ ID NO: 129 | GGGS | 4 |
| SEQ ID NO: 130 | GGGGS | 5 |
| SEQ ID NO: 131 | GGGSSGGS | 8 |
| SEQ ID NO: 132 | GGGGSGGGGS | 10 |
| SEQ ID NO: 133 | SGGSGGGGSGSG | 12 |
| SEQ ID NO: 134 | GGGSSGGSGGSGG | 13 |
| SEQ ID NO: 135 | GGGSSGGSGGSGGS | 14 |
| SEQ ID NO: 136 | GGGSSGGSGGSGGSG | 15 |
| SEQ ID NO: 137 | GGGSSGGSGGSGGSGGGSGGGGSG | 24 |
| SEQ ID NO: 138 | GGGSSGGSGGSGGGSSGGSGGSGGS | 25 |
| SEQ ID NO: 139 | GGGSGGGSSGGSGGSGGGGGSSGGS | 25 |
| SEQ ID NO: 140 | GGGSSGGSGGG | 11 |
| SEQ ID NO: 141 | GGGSSGGSGGSGGGSGGGS | 19 |
| SEQ ID NO: 142 | GGSG | 4 |
| SEQ ID NO: 143 | GGGSSGGSGGSGGSGGGSGGGS | 22 |
| SEQ ID NO: 144 | SGGGSGGGGSGGGGSGGGGSGGGSLQ | 25 |
| SEQ ID NO: 145 | GGGGSGGGGSGGGGS | 15 |
| SEQ ID NO: 146 | GGGGSGGGGSGGGGSGGGGS | 20 |
| SEQ ID NO: 147 | GGGGSSG | 7 |
| SEQ ID NO: 148 | GGGGSGGGGSGGGGSSGGSGGSGG | 24 |
| SEQ ID NO: 149 | SGGSGGGGS | 9 |
| SEQ ID NO: 150 | SGGSGG | 6 |

In other embodiments the linker contains a protease activatable (cleavable) moiety. In certain embodiment, the protease activatable moiety can be without limitation, the linkers in Table 4 or any combination thereof. Table 5 shows the placement of a protease activatable moieties in the context of representative constructs.

TABLE 4

| | | AA |
|---|---|---|
| SEQ ID NO: | SEQUENCE | Length |
| SEQ ID NO: 151 | ISSGLLSGRSDNH | 13 |
| SEQ ID NO: 152 | ISSGLLSGRSANP | 13 |
| SEQ ID NO: 153 | LSGRSDNH | 8 |
| SEQ ID NO: 154 | LSGRSANP | 8 |
| SEQ ID NO: 155 | PLGLAG | 6 |
| SEQ ID NO: 156 | IPVSLRSG | 8 |
| SEQ ID NO: 157 | GPQGIAGQ | 8 |
| SEQ ID NO: 158 | VPMSMRGG | 8 |
| SEQ ID NO: 159 | RPMSMIMG | 8 |
| SEQ ID NO: 160 | VPLSLTMG | 8 |
| SEQ ID NO: 161 | VPLSLYSG | 8 |
| SEQ ID NO: 162 | IPESLRAG | 8 |
| SEQ ID NO: 163 | IPVSLRSGWR | 10 |
| SEQ ID NO: 164 | IPVSLRSGRSA | 11 |
| SEQ ID NO: 165 | VPLSLYSGWR | 10 |
| SEQ ID NO: 166 | VPLSLYSGRSA | 11 |
| SEQ ID NO: 167 | GAANLVRG | 8 |
| SEQ ID NO: 168 | GYAELRMG | 8 |
| SEQ ID NO: 169 | MPYDLYHP | 8 |
| SEQ ID NO: 170 | RIGFLRTA | 8 |
| SEQ ID NO: 171 | ARYRWLTA | 8 |

TABLE 5

| ID No. | Construct design (cleavable linker underlined) |
|---|---|
| M123 | (IL-15RaSu)-Linker 1-IL15 (N72D)-CM(4-ISSGLLSGRSDNH (SEQ ID NO: 151)-8)- |
| | IL2R β(D1-D2)-Fc |
| M135 | (IL-15RaSu)-Linker 1-IL15 (N72D)-CM(4-IPVSLRSG (SEQ ID NO: 156)-13)-IL2R ß |
| | (D1-D2)-Fc |
| M141 | (IL-15RaSu)-Linker 1-IL15 (N72D)-CM(4-GPQGIAGQ (SEQ ID NO: 157)-13)-IL2R |
| | β(D1-D2)-Fc |
| M142 | (IL-15RaSu)-Linker 1-IL15 (N72D)-CM(4-VPMSMRGG (SEQ ID NO: 158)-13)- |
| | IL2R β(D1-D2)-Fc |
| M143 | (IL-15RaSu)-Linker 1-IL15 (N72D)-CM(4-RPMSMIMG (SEQ ID NO: 159)-13)- |
| | IL2R β(D1-D2)-Fc |
| M144 | (IL-15RaSu)-Linker 1-IL15 (N72D)-CM(4-VPLSLTMG (SEQ ID NO: 160)-13)-IL2R |
| | β(D1-D2)-Fc |
| M145 | (IL-15RaSu)-Linker 1-IL15 (N72D)-CM(4-VPLSLYSG (SEQ ID NO: 151)-13)-IL2R |
| | β(D1-D2)-Fc |
| M146 | (IL-15RaSu)-Linker 1-IL15 (N72D)-CM(4-IPESLRAG (SEQ ID NO: 161)-13)-IL2R |
| | β(D1-D2)-Fc |
| M162 | (IL-15RaSu)-Linker 1-IL15 (N72D)-CM(5-IPVSLRSGWR (SEQ ID NO: 163)-5)-IL2R |
| | β(D1-D2)-Fc |
| M166 | (IL-15RaSu)-Linker 1-IL15 (N72D)-CM(5-IPVSLRSGRSA (SEQ ID NO: 164)-5)-IL2R |
| | β(D1-D2)-Fc |
| M171 | (IL-15RaSu)-Linker 1-IL15 (N72D)-CM(4-GAANLVRG (SEQ ID NO: 167)-13)- |
| | IL2R β(D1-D2)-Fc |
| M172 | (IL-15RaSu)-Linker 1-IL15 (N72D)-CM(4-GYAELRMG (SEQ ID NO: 168)-13)- |
| | IL2R β(D1-D2)-Fc |
| M173 | IL2RbECD-CM(7-ARYRWLTA (SEQ ID NO: 171)-5)-IL15-linker 2-IL15Ra sushi-G4S- |
| | IgG1 Fc |
| M174 | IL2RbECD-CM(7-RIGFLRTA (SEQ ID NO: 170)-5)-IL15-linker 2-IL15Ra sushi-G4S- |
| | IgG1 Fc |
| M177 | (IL-15RaSu)-Linker 1-IL15 (N72D)-MMP9(4-VPLSLYSGWR (SEQ ID NO: 165)-12)- |
| | IL2R β(D1-D2)-Fc |
| M178 | (IL-15RaSu)-Linker 1-IL15 (N72D)-MMP9(4-VPLSLYSGRSA (SEQ ID NO: 166)- |
| | 12)-IL2R β(D1-D2)-Fc |

### Fc Region

In yet other aspects, the Fc region is altered by replacing at least one amino acid residue with a different amino acid residue to alter the effector functions. For example, one or more amino acids can be replaced with a different amino acid residue such that the Fc region has an altered affinity for an effector ligand. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the Fc region has altered C1q binding and/or reduced or abolished complement dependent cytotoxicity (CDC). This approach is described in, e.g., U.S. Pat. No. 6,194,551 by Idusogie et al.

In yet another aspect, one or more amino acid residues are changed to thereby alter the ability of the Fc region to fix complement. This approach is described in, e.g., the publication WO 94/29351 by Bodmer et al. In a specific aspect, one or more amino acids of an IL15 construct of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In another aspect, if a reduction of ADCC is desired, the Fc region of IgG4 was shown in many previous reports to have only modest ADCC and almost no CDC effector function (Moore G L, et al. 2010 MAbs, 2:181-189). However, natural IgG4 was found less stable in stress conditions such as in acidic buffer or under increasing temperature (Angal, S. 1993 Mol Immunol, 30:105-108; Dall'Acqua, W. et al, 1998 Biochemistry, 37:9266-9273; Aalberse et al. 2002 Immunol, 105:9-19). Reduced ADCC can be achieved by operably linking the IL5 construct to an IgG4 Fc engineered with combinations of alterations that reduce FcγR binding or C1q binding activities, thereby reducing or eliminating ADCC and CDC effector functions. Considering the physicochemical properties of an IL15 construct as a biological therapeutic, one of the less desirable, intrinsic properties of IgG4 is dynamic separation of its two heavy chains in solution (Van der Neut Kolfschoten M, et al. 2007 Science, 317:1554-157). The mutation of serine to proline at position 228 (EU numbering system) appeared inhibitory to the IgG4 heavy chain separation (Angal, S. 1993 Mol Immunol, 30:105-108; Aalberse et al. 2002 Immunol, 105:9-19). Some of the amino acid residues in the hinge and γFc region were reported to have impact on Fc region interaction with Fcγ receptors (Chappel S M, et al. 1991 Proc. Natl. Acad. Sci. USA, 88:9036-9040; Mukherjee, J. et al., 1995 FASEB J, 9:115-119; Armour, K. L. et al. 1999 Eur J Immunol, 29:2613-2624; Clynes, R. A. et al, 2000 Nature Medicine, 6:443-446; Arnold J. N., 2007 Annu Rev immunol, 25:21-50). Furthermore, some rarely occurring IgG4 isoforms in human population can also elicit different physicochemical properties (Brusco, A. et al. 1998 Eur J Immunogenet, 25:349-55; Aalberse et al. 2002 Immunol, 105:9-19). To generate IL15 constructs with low ADCC and CDC but with good stability, it is possible to modify the hinge and Fc region of human IgG4 and introduce a number of alterations. These modified IgG4 Fc molecules can be found in SEQ ID NOs: 83-88, U.S. Pat. No. 8,735,553 to Li et al.

The Fc domain can be modified via amino acid changes to provide “knob-into-hole” technology and to direct the pairing of two polypeptides together either in vitro or in vivo For example, “the knob-in-hole” mutations in the human IgG1 Fc were introduced to facilitate heterodimer formation (Ridgway et al., Prot. Eng. 1996 9:617-621). In addition, knob-into-holes were introduced in the Fc:Fc binding interfaces, CL:CHI interfaces or VH/VL interfaces of antibodies (see, e.g., US 2011/0287009, US2007/0178552, WO 96/027011, WO 98/050431, and Zhu et al, 1997, Protein Science 6:781-788). In some embodiments, knob-into-holes insure the correct pairing of two different heavy chains together to generate a specific IL15 construct.

IL15 Construct Production

The IL15 constructs can be produced by any means known in the art, including but not limited to, recombinant expression or chemical synthesis. Recombinant expression can be from any appropriate host cells known in the art, for example, mammalian host cells, bacterial host cells, yeast host cells, insect host cells, etc.

Also provided in the present disclosure are expression vectors and host cells for producing the IL15 constructs. The choice of expression vector depends on the intended host cells in which the vector is to be expressed. Typically, the expression vectors contain a promoter and other regulatory sequences (e.g., enhancers) that are operably linked to the polynucleotides encoding an IL15 construct. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of an IL15 construct. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the an IL15 construct can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express IL15 constructs. Insect cells in combination with baculovirus vectors can also be used.

In other aspects, mammalian host cells are used to express and produce the IL15 constructs of the present disclosure. For example, they can be a mammalian cell line harboring an exogenous expression vector. These include any normal mortal or normal or abnormal immortal animal or human cells. For example, several suitable host cell lines capable of secreting intact polypeptides have been developed, including the CHO cell lines, various COS cell lines and HEK 293 cells. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods of Treatment

The IL15 constructs of the present disclosure are useful in a variety of applications including, but not limited to, methods for the treatment of cancer, infection or immune disorders.

In one aspect, the present disclosure provides a method of treating cancer. In certain aspects, the method comprises administering to a patient in need an effective amount of an IL15 construct. The cancer can include, without limitation, gastric cancer, colon cancer, pancreatic cancer, breast cancer, head and neck cancer, kidney cancer, liver cancer, small cell lung cancer, non-small cell lung cancer, ovarian cancer, skin cancer, mesothelioma, lymphoma, leukemia, myeloma and sarcoma.

The IL15 constructs as disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

IL15 constructs of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The IL15 construct need not be, but is optionally formulated with one or more agents currently used to prevent or treat the disorder in question. The effective amount of such other agents depends on the amount of IL15 construct present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an IL15 construct of the disclosure will depend on the type of disease to be treated, the severity and course of the disease, whether the IL15 construct is administered for preventive or therapeutic purposes, previous therapy, the patient's clinical history and response to the IL15 construct, and the discretion of the attending physician. The IL15 construct is suitably administered to the patient at one time or over a series of treatments. Depending on the type and severity of the disease, about 1 μg/kg to 100 mg/kg of IL15 construct can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses can be administered intermittently, e.g. every week or every three weeks (e.g. such that the patient receives from about two to about twenty, or e.g. about six doses of the IL15 construct). An initial higher loading dose, followed by one or more lower doses can be administered. However, other dosage regimens can be useful. The progress of this therapy is easily monitored by conventional techniques and assays.

Combination Therapy

In one aspect, the IL15 constructs of the present disclosure can be used in combination with other therapeutic agents. Other therapeutic agents that can be used with the IL15 constructs of the present disclosure include: but are not limited to, a chemotherapeutic agent (e.g., paclitaxel or a paclitaxel agent; (e.g. Abraxane®), docetaxel; carboplatin; topotecan; cisplatin; irinotecan, doxorubicin, lenalidomide, 5-azacytidine, ifosfamide, oxaliplatin, pemetrexed disodium, cyclophosphamide, etoposide, decitabine, fludarabine, vincristine, bendamustine, chlorambucil, busulfan, gemcitabine, melphalan, pentostatin, mitoxantrone, pemetrexed disodium), tyrosine kinase inhibitor (e.g., erlotinib), multikinase inhibitor (e.g., sitravatinib), CD-20 targeting agent (e.g., rituximab, ofatumumab), CD52 targeting agent (e.g., alemtuzumab), prednisolone, lenalidomide, Bcl-2 inhibitor (e.g., oblimersen sodium), aurora kinase inhibitor, proteasome inhibitor (e.g., bortezomib), MEK inhibitor (e.g., ABT-348), JAK-2 inhibitor (e.g., INCB018424), mTOR inhibitor (e.g., temsirolimus, everolimus), BCR/ABL inhibitor (e.g., imatinib).

In one aspect, the IL15 construct of the present disclosure is administered in combination with an immune checkpoint agent. Without limitation, immune checkpoint agents can be PD-1. PD-L1, PD-L2, TIM3, LAG-3, OX40 or TIGIT antibodies. In one aspect, the anti-PDI antibody can be Tislelizumab. In one aspect, the anti-PDI antibody can be Ociperlimab or a combination of Tislelizumab and Ocipcrlimab.

In another aspect, IL15 has been administered in cell therapy, providing a beneficial effect to immune cells such as T-cells or NK cells when administered prior, during or after administration of cell therapy to a patient. For NK cells containing an anti-CD19 chimeric antigen receptor (CAR), an IL15 fusion transgene was introduced in order to support NK cell function and persistence (Kaufman et al. Blood 2018 v. 32, supp. 1, 4541). An EGFR CAR introduced into NK cells was administered in combination with an IL15 construct to promote efficacy in a glioblastoma model (Ma et al., Cancer Res., 2021 81(13) 3635-48). NK92 cells transduced with a CD123 CAR were designed to target acute myeloid leukemia (AML). Retroviral vectors were used to introduce a transgene cassette for the constitutive expression of human IL-15 which allowed for increased NK cell persistence in vivo (Morgan et al., Viruses 2021 13(7): 1365).

Pharmaceutical Compositions and Formulations

Also provided are compositions, including pharmaceutical formulations, comprising an IL15 construct. In certain embodiments, compositions comprise one or more IL15 constructs, or one or more polynucleotides comprising sequences encoding one or more IL15 constructs. These compositions can further comprise suitable carriers, such as pharmaceutically acceptable excipients including buffers, which are well known in the art.

Pharmaceutical formulations of an IL15 construct as described herein are prepared by mixing such IL15 construct having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

Exemplary lyophilized formulations are described in U.S. Pat. No. 6,267,958. Aqueous formulations include those described in U.S. Pat. No. 6,171,586 and WO2006/044908, the latter formulations including a histidine-acetate buffer.

Sustained-release preparations can be prepared. Suitable examples of sustained-release preparations include semipermeable matrices of solid hydrophobic polymers containing the IL15 construct, which matrices are in the form of shaped articles, e.g. films, or microcapsules. The formulations to be used for in vivo administration are generally sterile. Sterility can be readily accomplished, e.g., by filtration through sterile filtration membranes.

### EXAMPLES

### Example 1: Phosphoryl STAT5 Assessment in HH Cell Lines Treated with Different IL15 Constructs

HH cells are a human T lymphocyte/leukemia cell line that were obtained from ATCC (CRL-2105). Cultures were maintained by the addition or replacement of fresh medium. Cell cultures were started at 2×10⁵ cells/mL and maintain between 1×10⁵ and 1×10⁶ cells/mL, with culture medium be refreshed every 2-3 days. The IL15 used was unaltered IL15. P22339 is a published IL15 construct consisting of two molecules of IL15 linked to the sushi domain of IL15Ra and linked to an Fc (Hu et al. Sci. Rep. 2018 8:7675).

### IL-15 Stimulation

1. Cells were resuspend in PBS+0.5% BSA (Sigma, St. Louis MO) buffer and seeded into U 96-well plates (Corning™ #3799) at 5×10⁴ cells/25 μl/well.
2. The test plates were prepared in PBS (GIBCO #14190250, Gaithersburg MD)+0.5% BSA buffer as 6× solution starting with 1000 nM (final conc.) 4× serial dilution for 11 doses. Prepare solvent as vehicle.
3. Each test well received 5 μl prepared 6× compound solution and were incubated for 15 mins at 37° C. 5% CO₂.
   Phospho-STAT5 (Tyr694) was tested for using a HTRF Phospho-STAT5 (Tyr694) Cellular Assay Kit from CisBio™ (CisBio #64AT5PEG)
4. After cell treatment, 10 μL of supplemented lysis buffer (4×) was added and incubated for at least 60 minutes at room temperature under shaking.
5. Once the cells were lysed, 16 μL of cell lysate were transferred to a Cisbio 96-well HTRF detection plate (Cisbio 66PL96025), and 4 μL of pre-mixed HTRF antibodies were added to each well.
6. The plate was covered with a plate sealer and incubated overnight at room temperature.
7. The plate was read the fluorescence emission was taken at two different wavelengths (665 nm and 620 nm).

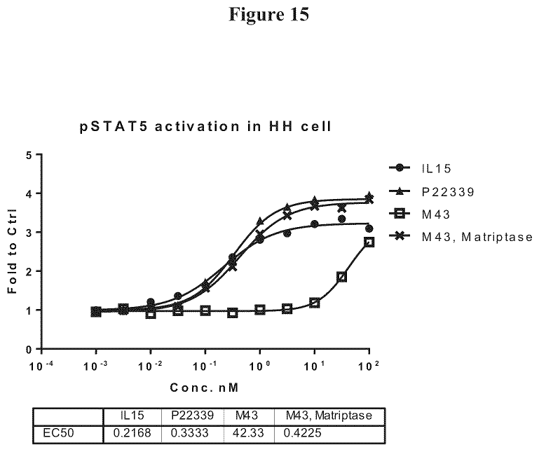

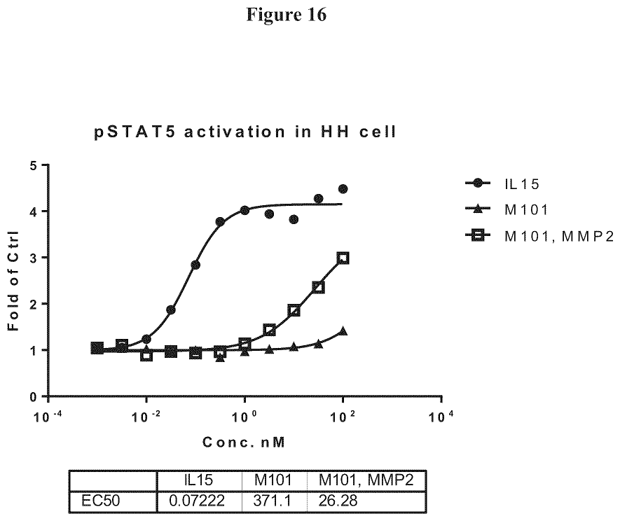

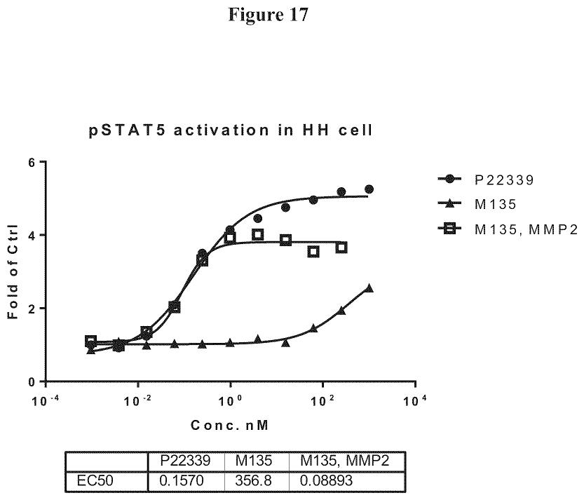

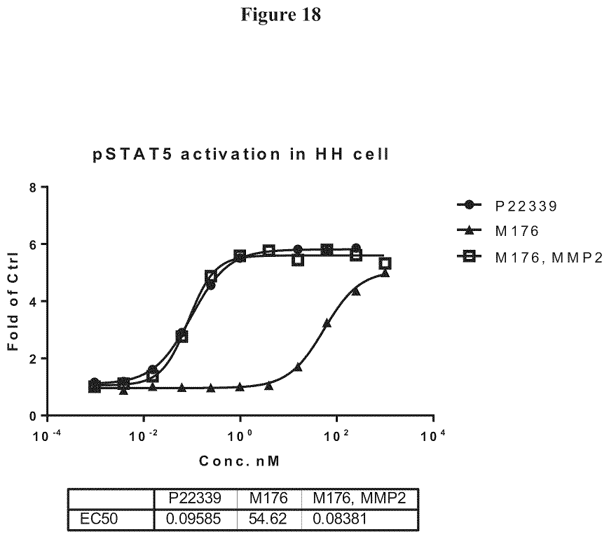

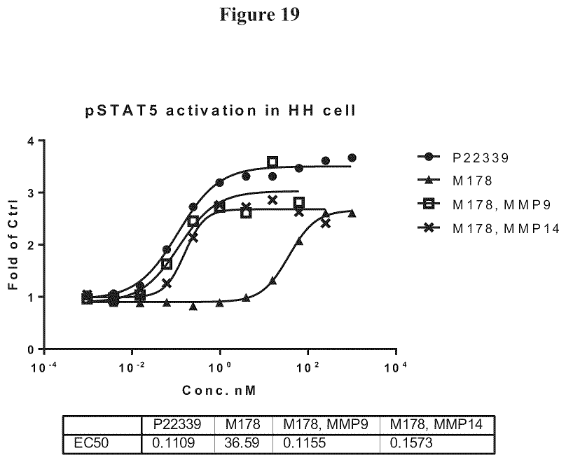

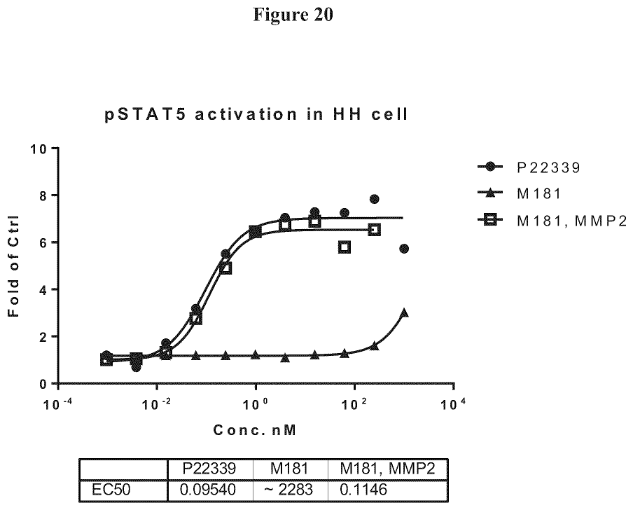

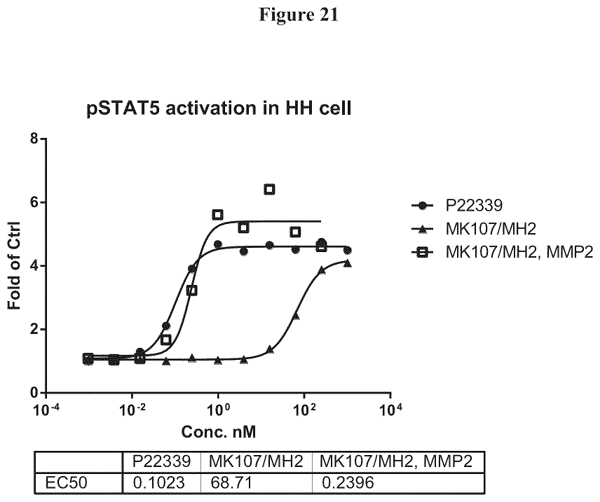

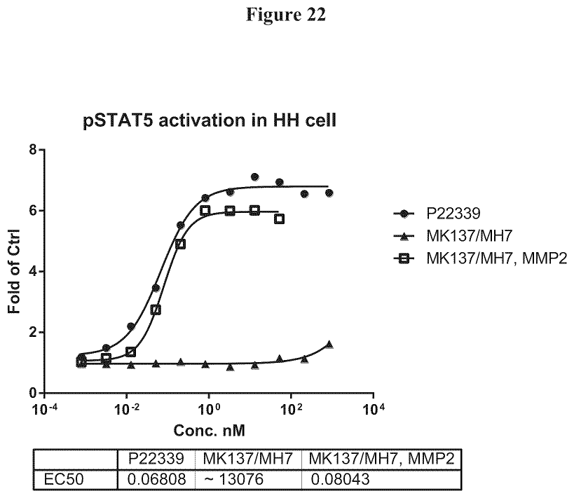

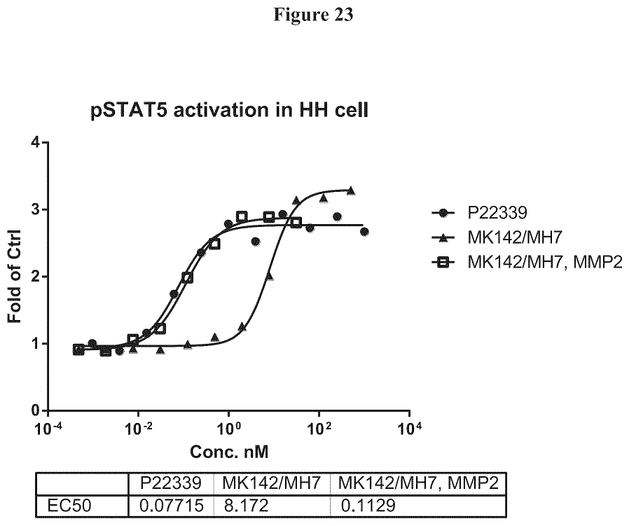

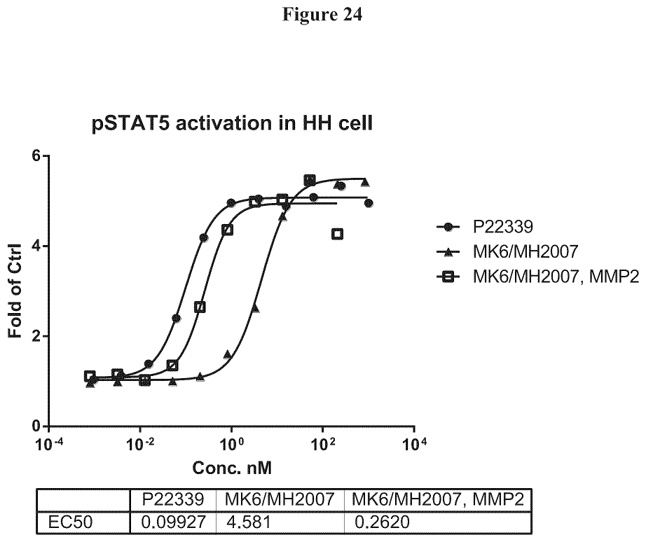

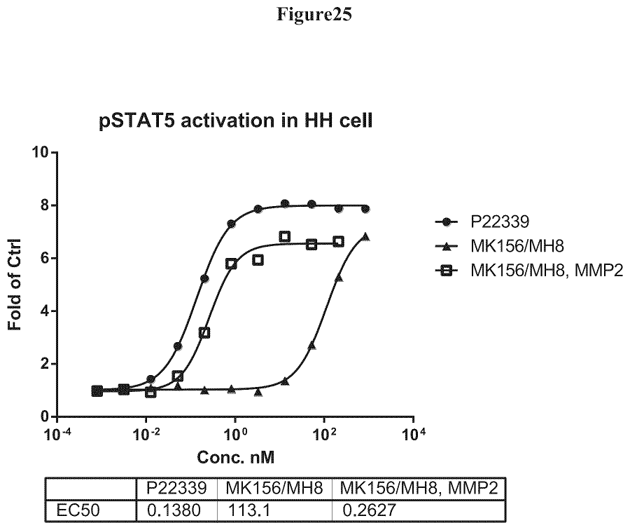

This assay provided insight as to the activity of specific constructs. In FIG. 15, the M43 construct, a bivalent Construct B, showed pSTAT5 activation on a similar level and curve with IL15 and P22339, in the presence of the cleaving protease, matriptase. In contrast, the non-matriptase cleaved M43 showed very little activity. The M101 construct, a bivalent Construct C showed very little activity prior to cleavage and after cleavage did not reach the levels of pSTAT5 activation demonstrated by IL15. This data is shown in FIG. 16. M135 which is of Construct A format, showed little activity when no matrix metalloprotease 2 (MMP2) protease was present. In the presence of MMP2, M135 showed activity similar to P22339 at low concentrations, but not at higher concentrations (FIG. 17). However, M176 (Construct A), demonstrated an activation curve very similar to P22339 when in the presence of MMP2 (FIG. 18). M178 (Construct A), demonstrated high activity in the presence of matrix metalloprotease 9 (MMP9) and a mid-range of activity in the presence of matrix metalloprotease 14 (MMP14), indicating that MMP14 was not as effective a protease as MMP9 in this specific construct (FIG. 19). M181 (Construct A) had very high pSTAT5 activity in the presence of MMP2 with very low activity when MMP2 was absent (FIG. 20). Using a different type of construct, MK107 (Construct E1) had very high pSTAT5 activity in the presence of MMP2 with very low activity when MMP2 was absent (FIG. 21). Similar results were seen with the MK137/MH7 construct, (Construct E3) with very low activity when no metalloprotease was present, but very high activity when MMP2 was present (FIG. 22). In FIG. 23 (MK142, Construct N) and FIG. 24 (MK6, Construct F3), the constructs showed partial activity when tested at high concentrations in the absence of a metalloprotease, but high activity at low concentrations in the presence of MMP2. MK156 (Construct N) showed good activity in the presence of MMP2 and activity at high concentrations in the absence of protease. This data is shown in FIG. 25.

### Example 2: M07e Cell Proliferation Assay

M07e Cells

M07e cells are human megakaryocyte line. The M07e cells were obtained from Nanjing CoBioer Biosciences Co., Ltd (COBIOER #CBP60791). The cell cultures were maintained in RPMI 1640 with 10% FBS and GM-CSF (10 ng/ml) or IL-2 (10 ng/nl) by addition or replacement of fresh medium. Assay cultures were started at 2×10⁵ cells/mL and maintained between 1×10⁵ and 1×10⁶ cells/mL. The M-07e cells proliferate in the presence of GM-CSF, IFN-alpha, IFN-beta, IFN-gamma, IL-2, IL-3, IL-4, IL-6, IL-15, NGF, SCF, TNT-alpha and TPO. IL15 reagents were obtained as described in Example 1. IL-2 was obtained from R&D Systems (#202-IL).

### IL-15 Stimulated Cell Proliferation

1. Cells were collected and resuspend in 1640+10% FBS without any cytokine, and seeded into 96-well plate for 0.8-1×10⁴ cells/90 μl/well.
2. Assay cultures were prepared in PBS+0.5% BSA buffer as 10× solution starting with 1000 nM (final conc.) 4× serial dilution for 9 doses. PBS was used as a diluent.
3. Dispense 10 μl prepared 10× compound solution to each test wells in duplicate. Incubate for 72-96 hours at 37° C. 5% CO₂.
4. A readout of luminescence provided an index of M07e proliferation when treated with P22339 or M181 construct with or without matriptase.

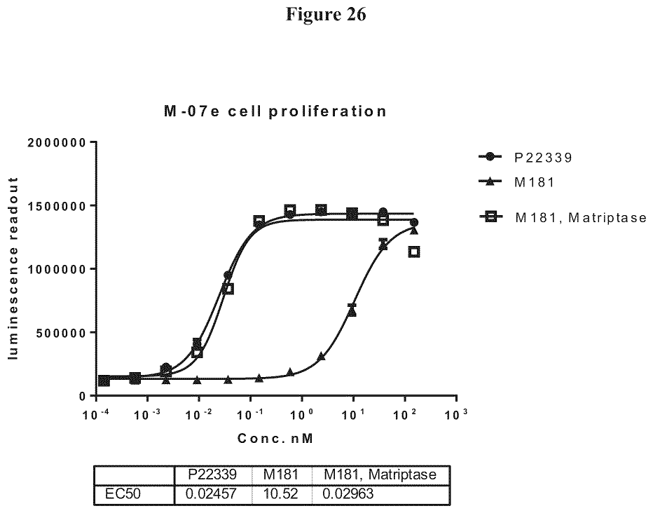

As shown in FIG. 26, the M181 construct (Construct A), had very little proliferative activity when not proteolytically treated. In contrast, when the M181 construct was treated with matriptase, the M181 construct displayed activity very similar to the P22339 IL15 construct.

### Example 3: Maximal Tolerated Dose Determination of MK137/MH7 in ICR Mice

ICR mice were obtained from Beijing Vital River Laboratory Animal Technology Co., Ltd. Female ICR mice were randomized into 13 groups according to body weight with 6 mice per group. Mice from 7 groups were intraperitoneally injected with single dose of vehicle (10 mM Histidine, 10 mM Acetic Acid, 240 mM sucrose, 0.02% Tween 20, pH5.5), or 0.3, 1 and 3 mg/kg P22339 or 10, 30 and 100 mg/kg MK137/MH7 for monitoring the general toxicological effect of each drug in ICR mice. Body weights were recorded every day and the mice were also monitored daily for clinical signs of toxicity throughout the study.

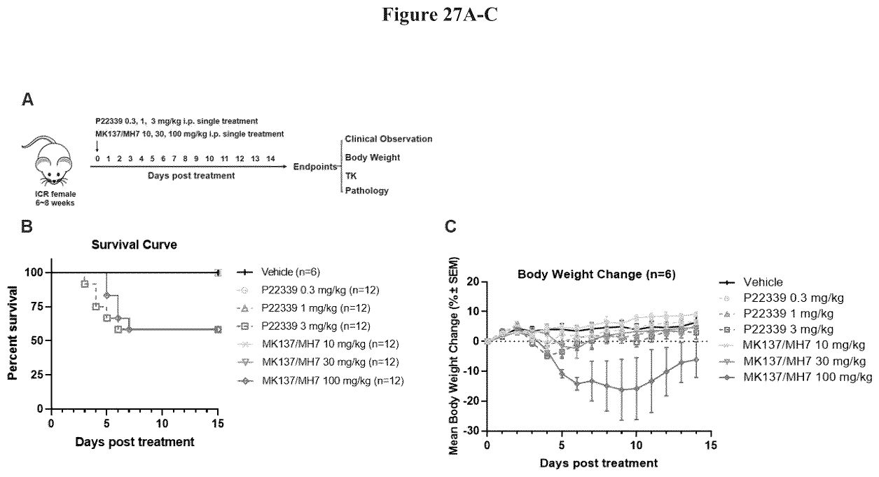

Mice from the other 6 groups were simultaneously intraperitoneally injected with single dose of 0.3, 1 and 3 mg/kg P22339 and 10, 30 and 100 mg/kg MK137/MH7 for evaluating the pharmacokinetics (PK) of each drug. A schematic of this is shown in FIG. 27A. Blood samples were collected from retro-orbital sinus under isoflurane/oxygen anesthesia at pre-dose and at 0.5, 2, 8, 24, 48, 72, 96, 120, 144 and 168 hours post dosing. Concentrations of P22339 and MK137/MH7 in serum at different doses and time points were measured by ELISA. Anti-human IgG1 Fc antibody is as capture antibody and anti-human IL15Ra antibody is as detection antibody.

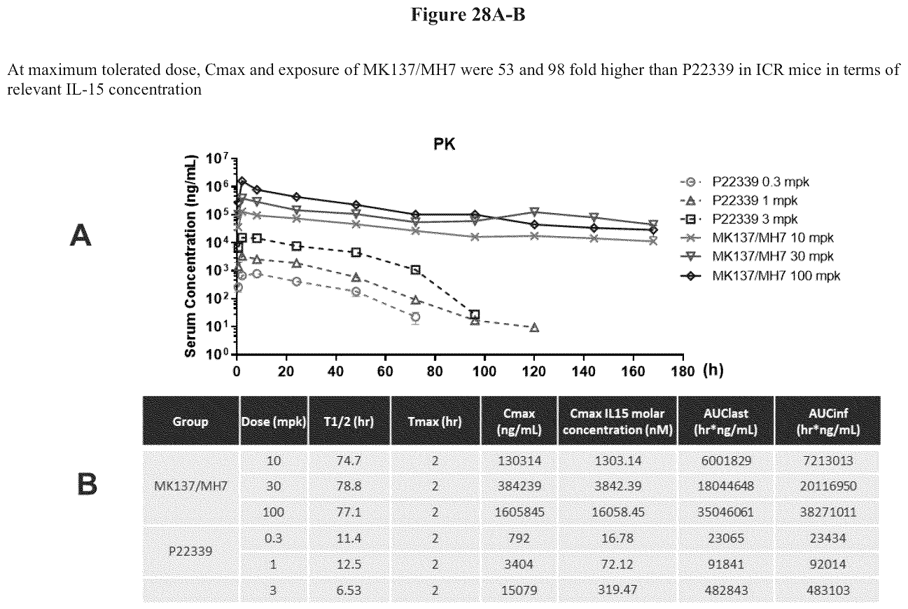

The survival time of all 78 mice was recorded to determine the maximal tolerated dose (MTD) of each drug. This result is shown in FIG. 27B, indicating that the mice treated with the MK137/MH7 construct have a better survival curve and limited body weight loss (FIG. 27C), demonstrating that the MK137/MH7 construct is better tolerated than P22339. Within FIG. 27B, the only mice that died were associated with dosing P22339 at 3 mpk and MK137/MH7 at 100 mpk, the rest of the doses were tolerated. From these results, the MTD as a single bolus injection of MK137/MH7 is 30 mg/kg, much higher than 1 mg/kg as the MTD for P22339. MK137/MH7 demonstrated a better PK profile with prolonged T1/2 and higher AUC than P22339, indicating that it has a much larger therapeutic window than P22339. These results are shown in FIG. 28A-B.

### Example 4: Dose-Dependent Pharmacodynamics Effects of MK137/MH7 on Peripheral Blood Cells and Tumor Infiltrated Lymphocytes

Female C57BL/6 mice were subcutaneously inoculated with 1×10⁷ GL261 cells. When the tumor volume reached around 100-200 mm³, animals were randomized according to the tumor volume with 7 animals per group. Mice were intraperitoneally injected with single dose of vehicle (10 mM Histidine, 10 mM Acetic Acid, 240 mM sucrose, 0.02% Tween 20, pH5.5) or 0.25 mg/kg P22339 or 10 and 30 mg/kg MK137/MH7. At 5 days post treatment, the mice were euthanized using carbon dioxide and 5 mice were selected for sample collection.

Blood samples were collected and used for further antibody staining directly. Tumor samples were minced using scissors into small pieces and followed by enzymatic digestion to isolate the tumor infiltrating lymphocytes (TILS). Blood cells and TILS were then stained with fluorescence conjugated antibody against certain biomarkers to identify NK cells, CD8⁺ T cells, CD4⁺ T cells and Treg cells for further analysis using flow cytometer.

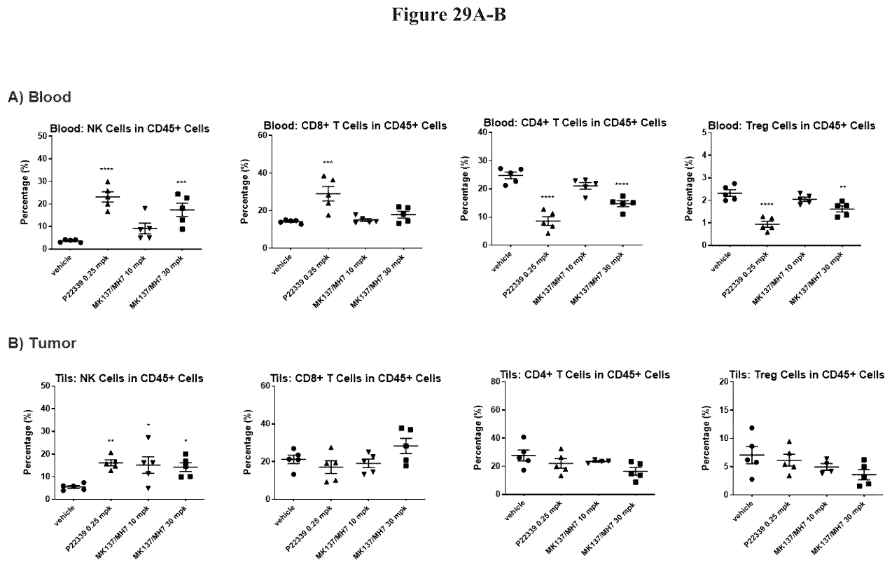

MK137/MH7 at 10 mpk showed significant in vivo PD effect in tils but not in peripheral blood cells regarding to the increase of NK percentage in lymphocytes. FIGS. 29A-B show the PD effects of P22339 and MK137/MH7 on peripheral blood cells and TILS. This data indicates that MK137/MH7 demonstrated significant PD effects in the tumor, but not in peripheral blood at 10 mpk indicating a large therapeutic window for MK137/MH7.

### Example 5: PK/PD Correlation of MK137/MH7 in an HT29+HH Xenograft Model

NCG mice are triple immunodeficient mice that lack functional T, B and NK cells and have reduced macrophage and dendritic cell function. Such characteristics makes these mice good models for research on immuno-oncology. Female NCG mice were obtained from Jiangsu GemPharmatech Co., Ltd., and were subcutaneously inoculated with a mix of 3×10⁶ HT29 (human colon adenocarcinoma) cells which have high expression of matrix metalloproteases and 1×10⁶ HH (human leukemia/lymphoma) cells which were chosen for their pSTAT5 response to IL15. Animals were randomized with 3 or 4 mice in each group when the average tumor size reached 400-600 mm³. A single dose of vehicle (10 mM Histidine, 10 mM Acetic Acid, 240 mM sucrose, 0.02% Tween 20, pH5.5), P22339, MK137/MH7 or MK138/MH7 (non-cleavable) were intraperitoneally administered to test the correlation of serum PK and IL15 induced tumor PD, in which pSTAT5 signaling from HH cells in HT29+HH tumor tissues was measured.

Serum intact MK137/MH7 and MK138/MH7 were detected by ELISA. Serum IL-15/IL-15Ra released from MK137/MH7 or MK138/MH7 were measured by MSD via their pSTAT5 induction level in HH cells which were spiked into serum obtained from NCG mice at different timepoints post treatment. Serum P22339 levels were examined by both ELISA and MSD as described. Tumor PD of MK137/MH7, MK138/MH7 and P22339 was evaluated by MSD at different timepoints post treatment by measuring pSTAT5 signaling from HH cells in HT29+HH tumor lysates.

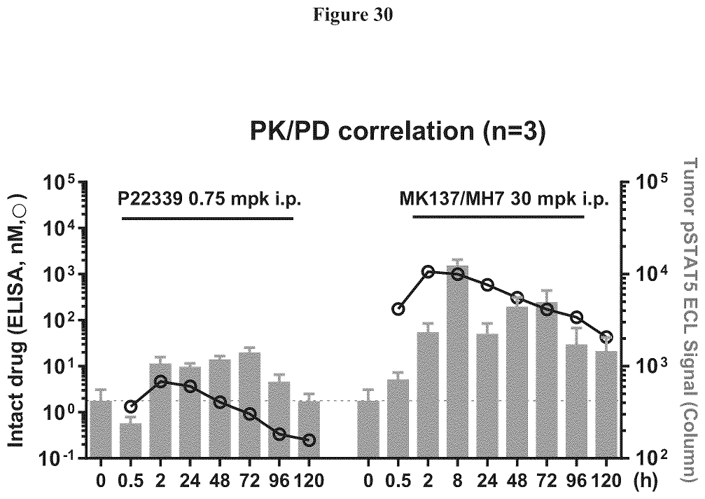

Using the maximum tolerated dose (MTD) as determined above in Example 3 using IRC mice, the PK/PD correlation of P22339 and MK138/MH7 in the HT29+HH xenograft model was tested. As shown in FIG. 30, MK138/MH7 is superior in PK/PD to the P22339 molecule demonstrating greater signaling and a greater therapeutic window.

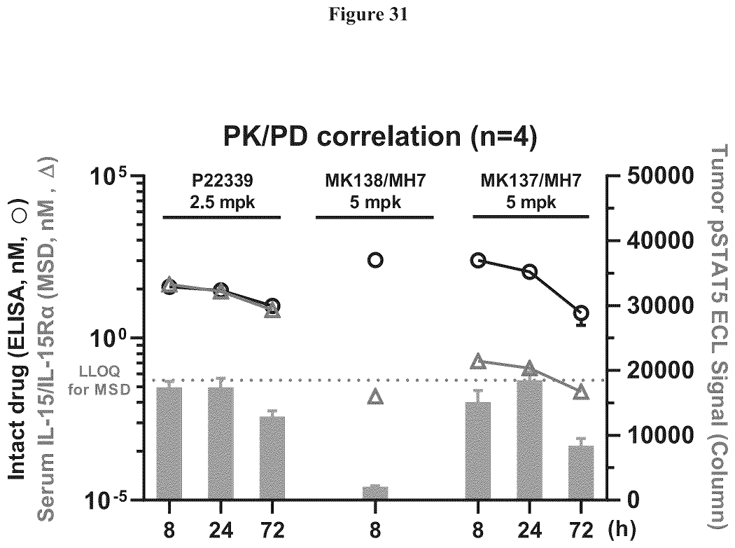

It is possible to calculate the equivalent amounts of molar IL15 between the MK137/MH7 and P22339 constructs, thus allowing these molecules to be dosed on that basis. When equivalent MK137/MH7 (5 mpk) and P22339 (2.5 mpk) doses were administered to the HT29+HH xenograft model, a similar PD was obtained in the tumor microenvironment as measured by ELISA (FIG. 31). However, when amounts of IL15 were measured in the serum of the mouse, MK137/MH7 release very little IL15 into the blood, as opposed to P22339. This indicates that MK137/MH7 has a better safety profile than P22339, as its activity is more restricted to the tumor, rather than systemic. As a control the non-cleavable MK138/MH7 had no activity in this model. According to HT29+HH model, 2.5 mpk P22339 showed similar PD with 5 mpk MK137/MH7. As demonstrated by the ICR mouse toxicity study in Example 3, the MTD for P22339 and MK137/MH7 is 1 mpk and 30 mpk, respectively. These data taken together indicates MK137/MH7 has a wider therapeutic window than P22339.

### Example 6: IL15 Constructs in Combination

Disclosed herein are a number of various IL15 constructs with cleavable linkers. It will be possible to create combinations of the IL15 constructs wherein one IL15 combined with at least one other IL15 construct as disclosed. This will account for differing amounts of protease expression in the tumor microenvironment. For example, a tumor expressing large amounts of a specific protease will cleave a protease activatable moiety in one IL15 construct very quickly, wherein a second protease that is not expressed as highly will cleave a second protease activatable moiety resulting in slower IL15 activation. If desired, this combination of IL15 constructs can allow for more construct delivered IL15 to remain in the tumor microenvironment longer. This can solve one of the issues associated with delivery of IL15, that of the very short half-life associated with systemic IL15 administration.

1. An interleukin 15 (IL15) construct comprising at least one IL15 molecule, at least one IL15 receptor alpha (IL15Ra) domain, at least one protease activatable moiety and at least one IgG1 Fc region.

2. The interleukin 15 (IL15) construct of claim 1, wherein the construct comprises a bivalent, homodimeric interleukin 15 (IL15) construct comprising from N-terminus to C-terminus:
a) an IL15 receptor alpha (IL15Ra) domain linked to;
b) a first linker (L1) linked to;
c) an IL15 domain, linked to;
d) a second linker (L2) containing a protease activatable moiety linked to;
e) an Interleukin 2 receptor beta (IL2Rb) domain; and
f) an IgG1 Fc region, wherein the bivalent IL15 construct comprises:
(i) a homodimer set forth in SEQ ID NO:4 (M123);
(ii) a homodimer set forth in SEQ ID NO:5 (M135);
(iii) a homodimer set forth in SEQ ID NO:6 (M140);
(iv) a homodimer set forth in SEQ ID NO:7 (M145);
(v) a homodimer set forth in SEQ ID NO:8 (M175);
(vi) a homodimer set forth in SEQ ID NO:9 (M176);
(vii) a homodimer set forth in SEQ ID NO:10 (M177);
(viii) a homodimer set forth in SEQ ID NO:11 (M178);
(ix) a homodimer set forth in SEQ ID NO:12 (M207);
(x) a homodimer set forth in SEQ ID NO:13 (M231);
(xi) a homodimer set forth in SEQ ID NO:14 (M233);
(xii) a homodimer set forth in SEQ ID NO:15 (M234);
(xiii) a homodimer set forth in SEQ ID NO:16 (M238);
(xiv) a homodimer set forth in SEQ ID NO:17 (M239);
(xv) a homodimer set forth in SEQ ID NO:18 (M240);
(xvi) a homodimer set forth in SEQ ID NO:19 (M241);
(xvii) a homodimer set forth in SEQ ID NO:20 (M243);
(xviii) a homodimer set forth in SEQ ID NO:21 (M244);
(xix) a homodimer set forth in SEQ ID NO:22 (M245);
(xx) a homodimer set forth in SEQ ID NO:23 (M246);
(xxi) a homodimer set forth in SEQ ID NO:24 (M247);
(xxii) a homodimer set forth in SEQ ID NO:25 (M248);
(xxiii) a homodimer set forth in SEQ ID NO:26 (M249);
(xxiv) a homodimer set forth in SEQ ID NO:27 (M327);
(xxv) a homodimer set forth in SEQ ID NO:28 (M328);
(xxvi) a homodimer set forth in SEQ ID NO:29 (M329);
(xxvii) a homodimer set forth in SEQ ID NO:30 (M330);
(xxviii) a homodimer set forth in SEQ ID NO:31 (M331); or
(xxix) a homodimer set forth in SEQ ID NO:32 (M332).

3. The interleukin 15 (IL15) construct of claim 1, wherein the construct comprises a bivalent, heterodimeric interleukin 15 (IL15) construct comprising from N-terminus to C-terminus:
a) an Interleukin 2 receptor beta (IL2Rb) domain linked to;
b) a first linker (L1) containing a protease activatable moiety, linked to;
c) an IL15 domain,
comprising a first molecule and a second molecule comprising from N-terminus to C-terminus:
x) an IL15 receptor alpha (IL15Ra) domain; and
y) an IgG1 Fc region, wherein the heterodimeric IL15 construct comprises:
(i) a first molecule set forth in SEQ ID NO:33 (M43) and a second molecule set forth in SEQ ID NO:34 (M24);
(ii) a first molecule set forth in SEQ ID NO:35 (M61) and a second molecule set forth in SEQ ID NO:36 (M60); or
(iii) a first molecule set forth in SEQ ID NO:37 (M62) and a second molecule set forth in SEQ ID NO:38 (M60).

4. The interleukin 15 (IL15) construct of claim 1, wherein the construct comprises a bivalent, homodimeric interleukin 15 (IL15) construct comprising from N-terminus to C-terminus:
a) an IgG1 Fc region, linked to;
b) a first linker (L1) linked to;
c) an Interleukin 2 receptor beta (IL2Rb) domain linked to;
d) a second linker (L2) containing a protease activatable moiety linked to;
e) an IL15 receptor alpha (IL15Ra) domain linked to;
f) a third linker (L3) linked to;
g) an IL15 domain; and
wherein the bivalent IL15 construct comprises:
(i) a homodimer set forth in SEQ ID NO:42 (M232)
(ii) a homodimer set forth in SEQ ID NO:43 (M1001);
(iii) a homodimer set forth in SEQ ID NO:44 (M1002);
(vi) a homodimer set forth in SEQ ID NO:45 (M1003);
(v) a homodimer set forth in SEQ ID NO:46 (M1004);
(vi) a homodimer set forth in SEQ ID NO:47 (M1005); or
(vii) a homodimer set forth in SEQ ID NO:48 (M1006).

5. The interleukin 15 (IL15) construct of claim 1, wherein the construct comprises a monovalent, heterodimeric interleukin 15 (IL15) construct comprising from N-terminus to C-terminus:
a) an IL15 receptor alpha (IL15Ra) domain linked to;
b) a first linker (L1) linked to;
c) an IL15 domain linked to;
d) a second linker (L2) containing a protease activatable moiety linked to;
e) an Interleukin 2 receptor beta (IL2Rb) domain; and
f) a first IgG1 Fc region,
as a first molecule and a second molecule comprising a second IgG1 Fc region, wherein the heterodimeric IL15 construct comprises a first molecule set forth in SEQ ID NO:49 (MK107) and a second molecule set forth in SEQ ID NO:50 (MH2).

6. The interleukin 15 (IL15) construct of claim 1, wherein the construct comprises a monovalent, heterodimeric interleukin 15 (IL15) construct comprising from N-terminus to C-terminus:
a) a first IgG1 Fc region linked to;
b) a first linker (L1) linked to;
c) an Interleukin 2 receptor beta (IL2Rb) domain linked to;
d) a second linker (L2) containing a protease activatable moiety to;
e) an IL15 receptor alpha (IL15Ra) domain linked to;
f) a third linker (L3) linked to;
g) an IL15 domain;
as a first molecule and a second molecule comprising a second IgG1 Fc region, wherein the monovalent, heterodimeric IL15 construct comprises:
(i) a first molecule set forth in SEQ ID NO:63 (M111) and a second molecule set forth in SEQ ID NO:64 (MH2);
(ii) a first molecule set forth in SEQ ID NO:65 (M2001) and a second molecule set forth in SEQ ID NO:52 (MH7); or
(iii) a first molecule set forth in SEQ ID NO:66 (M2002) and a second molecule set forth in SEQ ID NO:52 (MH7).

7. The interleukin 15 (IL15) construct of claim 1, wherein the construct comprises a monovalent, heterodimeric interleukin 15 (IL15) construct comprising from N-terminus to C-terminus:
a) an IL15 receptor alpha (IL15Ra) domain linked to;
b) a first linker (L1) linked to;
c) an IL15 domain linked to;
d) a second linker (L2) containing a protease activatable moiety linked to;
e) an Interleukin 2 receptor beta (IL2Rb) domain linked to;
f) a third linker (L3) linked to;
g) a first IgG1 Fc region,
as a first molecule and a second molecule comprising a second IgG1 Fc region, wherein the heterodimeric IL15 construct comprises:
(i) a first molecule set forth in SEQ ID NO:51 (MK114) and a second molecule set forth in SEQ ID NO:52 (MH7);
(ii) a first molecule set forth in SEQ ID NO:53 (MK115) and a second molecule set forth in SEQ ID NO:52 (MH7);
(iii) a first molecule set forth in SEQ ID NO:54 (MK117) and a second molecule set forth in SEQ ID NO:52 (MH7);
(iv) a first molecule set forth in SEQ ID NO:55 (MK118) and a second molecule set forth in SEQ ID NO:52 (MH7);
(v) a first molecule set forth in SEQ ID NO:56 (MK119) and a second molecule set forth in SEQ ID NO:52 (MH7);
(vi) a first molecule set forth in SEQ ID NO:57 (MK120) and a second molecule set forth in SEQ ID NO:52 (MH7);
(vii) a first molecule set forth in SEQ ID NO:58 (MK121) and a second molecule set forth in SEQ ID NO:52 (MH7);
(viii) a first molecule set forth in SEQ ID NO:59 (MK123) and a second molecule set forth in SEQ ID NO:52 (MH7);
(ix) a first molecule set forth in SEQ ID NO:60 (MK124) and a second molecule set forth in SEQ ID NO:52 (MH7);
(x) a first molecule set forth in SEQ ID NO:61 (MK125) and a second molecule set forth in SEQ ID NO:52 (MH7);
(xi) a first molecule set forth in SEQ ID NO:62 (MK126) and a second molecule set forth in SEQ ID NO:52 (MH7);
(xii) a first molecule set forth in SEQ ID NO:67 (MK136) and a second molecule set forth in SEQ ID NO:52 (MH7);
(xiii) a first molecule set forth in SEQ ID NO:68 (MK137) and a second molecule set forth in SEQ ID NO:52 (MH7);
(xiv) a first molecule set forth in SEQ ID NO:69 (MK138) and a second molecule set forth in SEQ ID NO:52 (MH7);
(xv) a first molecule set forth in SEQ ID NO:70 (MK139) and a second molecule set forth in SEQ ID NO:52 (MH7);
(xvi) a first molecule set forth in SEQ ID NO:71 (MK140) and a second molecule set forth in SEQ ID NO:52 (MH7);
(xvii) a first molecule set forth in SEQ ID NO:72 (MK141) and a second molecule set forth in SEQ ID NO:52 (MH7);
(xviii) a first molecule set forth in SEQ ID NO:73 (MK146) and a second molecule set forth in SEQ ID NO:52 (MH7);
(xix) a first molecule set forth in SEQ ID NO:74 (MK145) and a second molecule set forth in SEQ ID NO:75 (MH8);
(xx) a first molecule set forth in SEQ ID NO:76 (MK149) and a second molecule set forth in SEQ ID NO:75 (MH8);
(xxi) a first molecule set forth in SEQ ID NO:77 (MK150) and a second molecule set forth in SEQ ID NO:75 (MH8);
(xxii) a first molecule set forth in SEQ ID NO:78 (MK151) and a second molecule set forth in SEQ ID NO:75 (MH8);
(xxiii) a first molecule set forth in SEQ ID NO:79 (MK152) and a second molecule set forth in SEQ ID NO:75 (MH8);
(xxiv) a first molecule set forth in SEQ ID NO:80 (MK153) and a second molecule set forth in SEQ ID NO:75 (MH8);
(xxv) a first molecule set forth in SEQ ID NO:81 (MK154) and a second molecule set forth in SEQ ID NO:75 (MH8);
(xxvi) a first molecule set forth in SEQ ID NO:82 (MK155) and a second molecule set forth in SEQ ID NO:52 (MH7); or
(xxvii) a first molecule set forth in SEQ ID NO:172 (MK157) and a second molecule set forth in SEQ ID NO:75 (MH8).

8. The interleukin 15 (IL15) construct of claim 1, wherein the construct comprises a monovalent, heterodimeric interleukin 15 (IL15) construct comprising from N-terminus to C-terminus:
a) a first IgG1 Fc region linked to;
b) a first linker (L1) containing a protease activatable moiety linked to;
c) an IL15 receptor alpha (IL15Ra) domain linked to;
d) a second linker (L2) linked to;
e) an IL15 domain;
as a first molecule and a second molecule comprising:
x) a second IgG1 Fc region;
y) a linker (L3) linked to;
z) an Interleukin 2 receptor beta (IL2Rb) domain;
wherein the monovalent, heterodimeric IL15 construct comprises:
(i) a heterodimer set forth in SEQ ID NO:83 (M109) and set forth in SEQ ID NO:84 (MH110);
(ii) a heterodimer set forth in SEQ ID NO:85 (M2003) and set forth in SEQ ID NO:86 (MH2004); or
(iii) a heterodimer set forth in SEQ ID NO:87 (M2003) and set forth in SEQ ID NO:88 (MH2005).

9. The interleukin 15 (IL15) construct of claim 1, wherein the construct comprises a monovalent, heterodimeric interleukin 15 (IL15) construct comprising from N-terminus to C-terminus:
a) an Interleukin 2 receptor beta (IL2Rb) domain linked to;
b) a first linker (L1) containing a protease activatable moiety linked to;
c) an IL15 domain and;
d) a first IgG1 Fc region;
as a first molecule and a second molecule comprising:
x) an IL15 receptor alpha (IL15Ra) domain linked to;
y) a linker (L3) linked to;
z) a second IgG1 Fc region;
wherein the monovalent, heterodimeric IL15 construct comprises a first molecule set forth in SEQ ID NO:95 (M108) and a second molecule set forth in SEQ ID NO:96 (MH4).

10. The interleukin 15 (IL15) construct of claim 1, wherein the construct comprises a monovalent, heterodimeric interleukin 15 (IL15) construct comprising from N-terminus to C-terminus:
a) a first IgG1 Fc region linked to;
b) a first linker (L1) linked to;
c) an IL15 domain linked to;
d) a second linker (L2) containing a protease activatable moiety linked to;
e) an Interleukin 2 receptor beta (IL2Rb) domain;
as a first molecule and a second molecule comprising:
x) a second IgG1 Fc region linked to;
y) a linker (L3) linked to;
z) an IL15 receptor alpha (IL15Ra) domain;
wherein the monovalent, heterodimeric IL15 construct comprises:
a first molecule set forth in SEQ ID NO:97 (M112) and a second molecule set forth in SEQ ID NO:98 (MK113).

11. The interleukin 15 (IL15) construct of claim 1, wherein the construct comprises a bivalent homodimeric interleukin 15 (IL15) construct comprising from N-terminus to C-terminus:
a) a tumor associated antigen (TAA) binding antibody with a first IgG1 Fc region linked to;
b) a first linker (L1) linked to;
c) an Interleukin 2 receptor beta (IL2Rb) domain linked to;
d) a second linker (L2) containing a protease activatable moiety linked to;
e) an IL15 receptor alpha (IL15Ra) domain linked a third linker (L3) to;
f) an IL15 domain;
wherein the bivalent, homodimeric IL15 construct comprises the sequence set forth in SEQ ID NO:99 (M001) and the sequence set forth in SEQ ID NO:100 (MH333LC).

12. The interleukin 15 (IL15) construct of claim 1, wherein the construct comprises a monovalent heterodimeric interleukin 15 (IL15) construct comprising from N-terminus to C-terminus:
a) a tumor associated antigen (TAA) binding antibody with a first IgG1 Fc region linked to;
b) a first linker (L1) linked to;
c) an Interleukin 2 receptor beta (IL2Rb) domain linked to;
d) a second linker (L2) containing a protease activatable moiety linked to;
e) an IL15 receptor alpha (IL15Ra) domain linked to third linker (L3) linked to;
f) an IL15 domain;
wherein the monovalent, heterodimeric IL15 construct comprises the sequence set forth in SEQ ID NO:101 (M002), the sequence set forth in SEQ ID NO:102(MH2) and the sequence set forth in SEQ ID NO:100 (MH333LC).

13. The interleukin 15 (IL15) construct of claim 1, wherein the construct comprises a monovalent heterodimeric interleukin 15 (IL15) construct comprising from N-terminus to C-terminus:
a) a tumor associated antigen (TAA) binding antibody with a first IgG1 Fc region linked to;
b) a first linker (L1) containing a protease activatable moiety linked to;
c) an IL15 receptor alpha (IL15Ra) domain linked to;
d) a second linker (L2) linked to;
e) an IL15 domain;
wherein the monovalent, heterodimeric IL15 construct comprises the sequence set forth in SEQ ID NO:103 (MK3), and
x) a tumor associated antigen (TAA) binding antibody comprising a second IgG1 Fc region linked to;
y) a first linker (L3) linked to;
z) an Interleukin 2 receptor beta (IL2Rb) domain,
wherein the sequence is set forth in SEQ ID NO:104 (MH3) and is set forth in SEQ ID NO:100 (MH333LC).

14. The interleukin 15 (IL15) construct of claim 1, wherein the construct comprises a monovalent heterodimeric interleukin 15 (IL15) construct comprising from N-terminus to C-terminus:
a) a first tumor associated antigen (TAA) binding antibody with a first IgG1 Fc region linked to;
b) a first linker (L1) containing a protease activatable moiety linked to;
c) an IL15 domain linked to;
d) a second linker (L2) linked to;
e) an IL15 receptor alpha (IL15Ra) domain,
wherein the monovalent, heterodimeric IL15 construct comprises the sequence set forth in SEQ ID NO:105 (MK4), and
x) a first tumor associated antigen (TAA) binding antibody with a second IgG1 Fc region linked to;
y) a first linker (L3) set forth in;
z) an Interleukin 2 receptor beta (IL2Rb) domain,
wherein the sequence is set forth in SEQ ID NO:106 (MH3) and is set forth in SEQ ID NO:100 (MH333LC).

15. The interleukin 15 (IL15) construct of claim 1, wherein the construct comprises a monovalent, heterodimeric interleukin 15 (IL15) construct comprising from N-terminus to C-terminus:
a) an Interleukin 2 receptor beta (IL2Rb) domain linked to;
b) a first linker (L1) containing a protease activatable moiety linked to;
c) an IL15 domain;
as a first molecule; and
a second molecule comprising
x) an IL15 receptor alpha (IL15Ra) domain; and
y) a first IgG1 Fc region,
and a third molecule comprising a second IgG1 Fc region, wherein the heterodimeric IL15 construct comprises:
a first molecule set forth in SEQ ID NO:107 (MK143), a second molecule set forth in SEQ ID NO:108 (MK144) and a third molecule set forth in SEQ ID NO:52 (H7).

16. The interleukin 15 (IL15) construct of claim 1, wherein the construct comprises a monovalent, heterodimeric interleukin 15 (IL15) construct comprising from N-terminus to C-terminus:
a) a first IgG1 Fc region linked to;
b) a first linker (L1) linked to;
c) an IL15 receptor alpha (IL15Ra) domain linked to;
d) a second linker (L2) linked to;
e) an IL15 domain
f) a third linker (L3) containing a protease activatable moiety linked to;
g) an Interleukin 2 receptor beta (IL2Rb) domain;
as a first molecule and a second molecule comprising a second IgG1 Fc region, wherein the monovalent, heterodimeric IL15 construct comprises:
(i) a first molecule set forth in SEQ ID NO:109 (MK142) and a second molecule set forth in SEQ ID NO:52 (MH7);
(ii) a first molecule set forth in SEQ ID NO:173 (MK156) and a second molecule set forth in SEQ ID NO:75 (MH8); or
(iii) a first molecule set forth in SEQ ID NO:174 (MK165) and a second molecule set forth in SEQ ID NO:75 (MH8).

17. The interleukin 15 (IL15) construct of claim 1, wherein the construct comprises a monovalent, heterodimeric interleukin 15 (IL15) construct comprising from N-terminus to C-terminus:
a) an IL15 receptor alpha (IL15Ra) domain as a first molecule that is linked via a disulfide bond to;
b) an IL15 domain linked to;
c) a first linker (L1) containing a protease activatable moiety linked to;
d) an Interleukin 2 receptor beta (IL2Rb) domain linked to;
e) second linker (L2), linked to
f) a first IgG1 Fc region,
and a second molecule comprising a second IgG1 Fc region, wherein the heterodimeric IL15 construct comprises: a first molecule set forth in SEQ ID NO:110 (MK147) and a second molecule set forth in SEQ ID NO:111 (MK148) and a third molecule set forth in SEQ ID NO:52 (MH7).

18. The interleukin 15 (IL15) construct of claim 1, wherein the construct comprises a monovalent heterodimeric interleukin 15 (IL15) construct comprising from N-terminus to C-terminus:
a) a tumor associated antigen (TAA) binding antibody with a first IgG1 Fc region linked to;
b) a first linker (L1) linked to;
c) an IL15 receptor alpha (IL15Ra) domain linked to;
d) a second linker (L2) linked to;
e) an IL15 domain linked to;
f) a third linker (L3) containing a protease activatable moiety linked to;
g) an Interleukin 2 receptor beta (IL2Rb) domain; and
wherein the monovalent, heterodimeric IL15 construct comprises the sequence set forth in SEQ ID NO:175(MK14), the sequence set forth in SEQ ID NO:102(MH2) and the sequence set forth in SEQ ID NO:100(MH333LC).

19. A pharmaceutical composition comprising the IL15 construct of claim 1 in combination with at least one additional IL15 construct.

20. A method of treating cancer comprising administering to a patient in need an effective amount of the IL15 construct of claim 1.

21. The method of claim 20, wherein the cancer is gastric cancer, colon cancer, pancreatic cancer, breast cancer, head and neck cancer, kidney cancer, liver cancer, small cell lung cancer, non-small cell lung cancer, ovarian cancer, skin cancer, mesothelioma, lymphoma, leukemia, myeloma and sarcoma.

22. The method of claim 20, wherein the IL15 construct is administered in combination with another therapeutic agent.

23. The method of claim 22, wherein the therapeutic agent is an immune checkpoint agent.

24. The method of claim 23, wherein the immune checkpoint agent is a PD-1, PD-L1, PD-L2, TIM3, LAG-3, OX40 or TIGIT antibody.

25. A method of increasing the survival of an immune cell, comprising administering an IL15 construct of claim 1 prior to, during or after administration of an effective amount of immune cells to a patient.

26. The method of claim 25 wherein the immune cell expresses a chimeric antigen receptor (CAR).

27. The method of claim 26 wherein the immune cell is an NK cell or a T-cell.

28. (canceled)
